(19) United States
(12) Patent Application Publication
(10) Pub. No.: US 2025/0268994 A1
(43) Pub. Date: Aug. 28, 2025

(54) Optimized Nucleotide Sequences Encoding the Extracellular Domain of Human ACE2 Protein or a Portion Thereof

(71) Applicant: TRANSLATE BIO, INC., Waltham, MA (US)

(72) Inventors: Lianne BOEGLIN, Hollis, NH (US); Frank DEROSA, Chelmsford, MA (US); Anusha DIAS, Medfield, MA (US); Joseph A. SKALESKI, Woburn, MA (US); Nicholas CLARK, Billerica, MA (US); Richard WOOSTER, Glasgow (GB); Khang Anh TRAN, Quincy, MA (US); Shrirang KARVE, Acton, MA (US)

(73) Assignee: TRANSLATE BIO, INC., Waltham, MA (US)

(21) Appl. No.: 18/552,165

(22) PCT Filed: Mar. 25, 2022
(86) PCT No.: PCT/US2022/022006

§ 371 Date: Sep. 22, 2023

Related U.S. Application Data
(60) Provisional application No. 63/166,119, filed on Mar. 25, 2021.
(60) Provisional application No. 63/220,271, filed on Jul. 9, 2021.

(51) Int. Cl. A61K 38/48; A61P 31/14; C12N 9/48

(52) U.S. Cl. CPC A61K 38/4813; A61P 31/14; C12N 9/485; C12Y 304/17023; C07K 2319/30

(57) ABSTRACT

The present invention provides compositions that are useful in treating or preventing an infection in a subject caused by a virus which uses human angiotensin-converting enzyme 2 (ACE2) protein for cellular entry. Such compositions act by neutralizing the virus, preventing it from binding to the human ACE2 protein and gaining cellular entry. In particular, the invention provides an mRNA comprising an optimized nucleotide sequence encoding a polypeptide comprising the extracellular domain of ACE2 protein or a portion thereof, which binds to a viral surface protein. The mRNA may be encapsulated in lipid nanoparticles (LNPs) for delivery to a subject in vivo. Typically, compositions comprising the LNPs are delivered to a subject either intravenously or as an aerosol, for example via nebulization.

### CROSS-REFERENCE TO RELATED APPLICATIONS

The present application is a national stage application under 35 U.S.C. § 371 of International Patent Application No. PCT/US2022/022006, filed on Mar. 25, 2022, which claims priority to U.S. Provisional Application No. 63/166,119, filed on Mar. 25, 2021, and U.S. Provisional Application No. 63/220,271, filed on Jul. 9, 2021. The contents of all the aforementioned applications are incorporated herein by reference in their entirety.

### SEQUENCE LISTING

The present specification makes reference to a Sequence Listing (submitted electronically as a .txt file named “MRT-2216WO_SL” on Mar. 25, 2022). The .txt file was generated on Mar. 21, 2022 and is 132 KB in size. The entire contents of the sequence listing are herein incorporated by reference.

### FIELD OF THE INVENTION

The present invention relates to polypeptides comprising the extracellular domain of human angiotensin-converting enzyme 2 (ACE2) protein or a portion thereof, mRNAs comprising optimized nucleotide sequences encoding such polypeptides, and lipid nanoparticles (LNPs) encapsulating such mRNAs. These polypeptides, mRNAs, and LNPs are particularly suitable for use in compositions for treating or preventing an infection in a subject caused by a virus which uses human ACE2 protein for cellular entry, and for neutralizing such a virus in a subject.

### BACKGROUND OF THE INVENTION

Viral pandemics pose a serious threat to global public health. In recent years, viruses which use the human angiotensin-converting enzyme 2 (ACE2) receptor for cellular entry have caused at least one widespread epidemic involving severe acute respiratory syndrome (SARS), and one ongoing global pandemic involving Coronavirus Disease 2019 (COVID-19). The causative agent of SARS is SARS coronavirus (SARS-CoV), and the causative agent of COVID-19 is SARS coronavirus 2 (SARS-CoV-2). Infections caused by viruses, which rely on binding to the ACE2 receptor to enter human cells, can cause acute pneumonia, respiratory distress, and other organ damage in subjects and are associated with high mortality, in particular in subjects above 50 years of age.

Subjects can be infected with SARS-CoV and SARS-CoV-2 through direct contact with contaminated surfaces, direct contact with respiratory droplets of an infected person, and in particular, through airborne transmission. SARS-CoV and SARS-CoV-2 frequently establish infection through binding to the ACE2 receptors present on mucosal or airway epithelial cells of subjects, entering mucosal or airway epithelial cells, and commencing viral replication. No vaccine exists to date for SARS-CoV, and its natural reservoir has not been identified. Several monovalent vaccines against SARS-CoV-2 have been demonstrated to be effective in preventing COVID-19, but robust and long-lasting immune responses may be elusive due to immune evasion by viral mutation. There is also an acute lack of sufficient and effective treatment options for subjects with COVID-19. Antibody cocktails against SARS-CoV-2 antigens may be rendered ineffective by viral mutation, and other drugs such as remdesivir or dexamethasone do not completely treat COVID-19 symptoms in a substantial portion of cases.

Given that SARS-CoV and SARS-CoV-2 rely on the ACE2 receptor to enter and infect cells, it has been proposed that administering ‘decoy receptors’ comprising the soluble portion of the ACE2 protein to subjects can block the binding of the viral surface spike (S) glycoprotein to the genuine ACE2 receptors present on the surface of mucosal or airway epithelial cells. Such approaches which block the binding of the virus to its receptor for cellular entry can either prevent primary infection, or block spread of the virus, effectively neutralizing the virus encountered by a subject. Such approaches are advantageous over treatments of the virus itself because they remain effective even if the virus mutates. Previous approaches to use ‘decoy receptors’ involved expressing soluble recombinant ACE2 proteins, and administering the recombinant proteins to patients intravenously or by inhalation into the lungs (Zoufaly et al., Lancet Respir Med. 2020 November; 8(11), pp. 1154-1158; Monteil et al., Cell. 2020 May 14; 181(4):905-913.e7.; Ameratunga et al., N Z Med J. 2020 May 22; 133(1515):112-118). At lower expression levels, soluble ACE2 proteins have been reported to facilitate endocytosis and cellular entry of SARS-CoV-2 in a human kidney cell line via receptors involved in the renin-angiotensin system (Yeung et al., Cell. 2021 Mar. 2; 50092-8674(21)00283-X. doi: 10.1016/j.cell.2021.02.053). However, high levels of soluble ACE2 protein inhibit SARS-CoV-2 infection and may saturate the receptors involved in endocytic recycling of the ACE2 receptor, and effectively neutralize the viral particles. Previous recombinant protein-based approaches are laborious and costly and do not scale easily in a disease outbreak. Attempts have been made to increase the affinity between the ‘decoy’ ACE2 receptors and the viral surface protein by mutation and screening (Linsky et al., Science. 2020 Dec. 4; 370(6521): pp. 1208-1214; Chan et al., Science. 2020 Sep. 4; 369(6508): pp. 1261-1265; Glasgow et al., Proc Natl Acad Sci USA. 2020 Nov. 10; 117(45): pp. 28046-28055). Others have explored the rapid generation of circulating and mucosal ACE2 using mRNA nanotherapeutics for the potential treatment of SARS-CoV-2 (Kim et al., bioRxiv 2020.07.24.205583; doi: 10.1101/2020.07.24.205583).

mRNA therapy is increasingly important for treating various diseases, especially those caused by dysfunction of proteins or genes. mRNA therapy can restore the normal levels of an endogenous protein, or provide an exogenous therapeutic protein without permanently altering the genome sequence or entering the nucleus of the cell. mRNA therapy takes advantage of the cell's own protein production and processing machinery to treat diseases or disorders, is flexible to tailored dosing and formulation, and is broadly applicable to any disease or condition caused by an underlying gene or protein defect or treatable through the provision of an exogenous protein.

Expression levels of an mRNA-encoded protein can significantly impact the efficacy and therapeutic benefits of mRNA therapy. Effective expression or production of a protein from an mRNA within a cell depends on a variety of factors, including the route of delivery, the property of the delivery vehicle and the sequence of the mRNA itself. For example, optimization of the composition and order of codons within a protein-coding nucleotide sequence (“codon optimization”) can lead to higher expression of the mRNA-encoded protein. Similarly, the route of delivery may be important to ensure effective expression in target tissues particularly affected by infection with the various. The choice of the delivery vehicle can make cellular uptake more efficient.

Accordingly, a need exists for optimized therapeutic approaches which optimize delivery and expression of mRNA encoding a soluble version of the ACE2 protein for treating or preventing an infection caused by a virus which uses human ACE2 protein for cellular entry.

### SUMMARY OF THE INVENTION

The inventors have discovered that mRNAs comprising an optimized nucleotide sequence encoding a polypeptide comprising the extracellular domain of human ACE2 protein are particularly useful in increasing the level and duration of expression of soluble versions of the ACE2 protein, making such mRNAs particularly useful in methods of treating or preventing an infection caused by a virus which uses human ACE2 protein for cellular entry in a subject. The present invention also provides optimized lipid nanoparticles (LNPs) which facilitate delivery and expression of the mRNAs of the invention, along with related compositions, kits, and formulations for storing and delivering LNPs encapsulating the sequence-optimized mRNAs of the invention.

Accordingly, the invention provides an mRNA comprising an optimized nucleotide sequence encoding a polypeptide comprising the extracellular domain of human angiotensin-converting enzyme 2 (ACE2) protein or a portion thereof, which binds to a viral surface protein. In a typical embodiment of the invention, an optimized nucleotide sequence in accordance with the invention contains one or more of the following modifications relative to a naturally occurring nucleotide sequence encoding the human ACE2 protein or a portion thereof. For example, the coding region of an optimized nucleotide sequence of the invention usually consists of codons associated with a usage frequency which is greater than or equal to 10%. An optimized nucleotide sequence in accordance with the invention typically (a) does not contain a termination signal having one of the following nucleotide sequences: 5′-X₁AUCUX₂UX₃-3′, wherein X₁, X₂ and X₃ are independently selected from A, C, U or G; and 5′-X₁AUCUX₂UX₃-3′, wherein X₁, X₂ and X₃ are independently selected from A, C, U or G; (b) does not contain any negative cis-regulatory elements and negative repeat elements; and (c) has a codon adaptation index (CAI) greater than 0.8. When divided into non-overlapping 30 nucleotide-long portions, each portion of an optimized nucleotide sequence in accordance with the invention commonly has a guanine cytosine content ranging from 30%-70%.

In a specific embodiment, an optimized nucleotide sequence in accordance with the invention does not contain a termination signal having one of the following sequences: UAUCUGUU; UUUUUU; AAGCUU; GAAGAGC; UCUAGA.

In a particular embodiment, the extracellular domain of human ACE2 protein or portion thereof comprises amino acid residues 1-740 of the naturally occurring human ACE2 protein encoded by SEQ ID NO: 10. In certain embodiments, the polypeptide encoded by the mRNA of the invention may comprise two extracellular domains of human ACE2 protein or portions thereof operably linked to form a dimer.

In particular embodiments, the polypeptide encoded by the mRNA of the invention comprises an immunoglobulin Fc region linked to the C-terminus of the extracellular domain of human ACE2 protein or portion thereof. The immunoglobulin Fc region can be derived from IgG1, IgG2, or IgG4. In a specific embodiment, the immunoglobulin Fc region is derived from human IgG1.

In some embodiments, the polypeptide encoded by the mRNA of the invention comprises a C-terminal localization sequence which is exogenous to the naturally occurring human ACE2 protein encoded by SEQ ID NO: 10. In particular embodiments, the polypeptide comprises the C-terminal localization sequence and an immunoglobulin Fc region.

In particular embodiments, the extracellular domain of human ACE2 protein or portion thereof, C-terminal localization sequence, and/or immunoglobulin Fc region within the polypeptide are linked by one or more linker sequences, e.g., GGGGS. Exemplary polypeptides containing such linkers are described in more detail below.

In specific embodiments, the extracellular domain of human ACE2 protein or portion thereof contains one or more amino acid substitutions, relative to a naturally occurring human ACE2 protein encoded by the amino acid sequence of SEQ ID NO: 10. The one or more amino acid substitutions may increase the binding affinity for SARS-CoV-2 spike (S) glycoprotein. In some embodiments, the one or more amino acid substitutions inactivate the catalytic activity of human ACE2 protein. In other embodiments, the one or more amino acid substitutions affect the glycosylation of human ACE2 protein. In particular embodiments, the one or more amino acid substitutions is at any one of the following amino acid positions of the naturally occurring human ACE2 protein encoded by the amino acid sequence of SEQ ID NO: 10: positions 90, 92, 273, 374, and 378. In some embodiments, the one or more amino acid substitutions are selected from the group consisting of: N90D, T92A, R273A, H374N, and H378N. In one specific embodiment, the one or more amino acid substitutions comprise or are N90D and R273A. In another specific embodiment, the one or more amino acid substitutions comprise or are N90D and T92A. In yet another specific embodiment, the one or more amino acid substitutions comprise or are H374N and H378N.

In some embodiments, the amino acid sequence of the polypeptide encoded by the mRNA of the invention is at least 90%, 95%, 96%, 97%, 98%, or 99% identical to the amino acid sequence of any one of SEQ ID NOs: 11 to 18, 34 and 35. In particular embodiments, the amino acid sequence of the polypeptide comprises or consists of the amino acid sequence of any one of SEQ ID NOs: 11 to 18, 34 and 35. In a specific embodiment, the amino acid sequence of the polypeptide comprises or consists of the amino acid sequence of SEQ ID NO: 11. In a specific embodiment, the amino acid sequence of the polypeptide comprises or consists of the amino acid sequence of SEQ ID NO: 12. In a specific embodiment, the amino acid sequence of the polypeptide comprises or consists of the amino acid sequence of SEQ ID NO: 13. In a specific embodiment, the amino acid sequence of the polypeptide comprises or consists of the amino acid sequence of SEQ ID NO: 14. In a specific embodiment, the amino acid sequence of the polypeptide comprises or consists of the amino acid sequence of SEQ ID NO: 15. In a specific embodiment, the amino acid sequence of the polypeptide comprises or consists of the amino acid sequence of SEQ ID NO: 16. In a specific embodiment, the amino acid sequence of the polypeptide comprises or consists of the amino acid sequence of SEQ ID NO: 17. In a specific embodiment, the amino acid sequence of the polypeptide comprises or consists of the amino acid sequence of SEQ ID NO: 18. In a specific embodiment, the amino acid sequence of the polypeptide comprises or consists of the amino acid sequence of SEQ ID NO: 34. In a specific embodiment, the amino acid sequence of the polypeptide comprises or consists of the amino acid sequence of SEQ ID NO: 35.

In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 2 and encodes the amino acid sequence of SEQ ID NO: 11. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 3 and encodes the amino acid sequence of SEQ ID NO: 12. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 4 and encodes the amino acid sequence of SEQ ID NO. 13. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 5 and encodes the amino acid sequence of SEQ ID NO. 14. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 6 and encodes the amino acid sequence of SEQ ID NO. 15. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 7 and encodes the amino acid sequence of SEQ ID NO. 16. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 8 and encodes the amino acid sequence of SEQ ID NO. 17. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 9 and encodes the amino acid sequence of SEQ ID NO. 18. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 32 and encodes the amino acid sequence of SEQ ID NO. 34. In some embodiments, an optimized nucleotide sequence in accordance with the invention is at least 60%, 65%, 70%, 75%, 80%, 85%, 90%, 95%, 96%, 97%, 98%, or 99% identical to, comprises, or consists of the nucleic acid sequence of SEQ ID NO: 33 and encodes the amino acid sequence of SEQ ID NO. 35.

In a specific embodiment, the mRNA of the invention comprises a 5′ cap with the following structure:

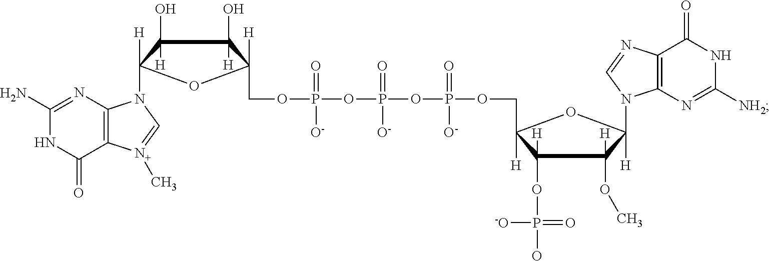

In one specific embodiment, the mRNA of the invention comprises a poly(A) tail. In another specific embodiment, the mRNA of the invention comprises a poly(C) tail. In some embodiments, the tail comprises at least 50 adenosine or cytosine nucleotides. In a typical embodiment, the tail is approximately 100-500 nucleotides in length.

In some embodiments, the mRNA of the invention comprises a 5′ untranslated region (5′ UTR) different than the naturally occurring 5′ UTR of a human ACE2 mRNA. In a specific embodiment, the 5′ UTR has the nucleotide sequence of SEQ ID NO: 19.

In some embodiments, mRNA of the invention comprises a 3′ untranslated region (3′ UTR) different than the naturally occurring 3′ UTR of a human ACE2 mRNA. In a specific embodiment, the 3′ UTR has the nucleotide sequence of SEQ ID NO: 20 or SEQ ID NO: 21.

In some embodiments, the mRNA of the invention comprises one or more modified nucleosides. The one or more modified nucleosides may be a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N-1-methyl-pseudouridine), 2-thiouridine, and 2-thiocytidine.

The invention also provides a DNA vector encoding the mRNA of the invention. The DNA vector may comprise a promoter and/or a terminator. In one embodiment, the promoter is a SP6 RNA polymerase promoter. In another embodiment, the promoter is a T7 RNA polymerase promoter.

The invention also provides a lipid nanoparticle (LNP) encapsulating the mRNA of the invention. The lipid component of the LNP typically consists of a cationic lipid, a non-cationic lipid, a PEG-modified lipid, and optionally a cholesterol-based lipid. The cationic lipid may be selected from cKK-E12, cKK-E10, HGT5000, HGT5001, ICE, HGT4001, HGT4002, HGT4003, TL1-01D-DMA, TL1-04D-DMA, TL1-08D-DMA, TL1-10D-DMA, OF-Deg-Lin, OF-02, GL-TES-SA-DMP-E18-2, GL-TES-SA-DME-E18-2, SY-3-E14-DMAPr, HEP-E3-E10, HEP-E4-E10, RL3-DMA-07D, RL2-DMP-07D, cHse-E-3-E10, cHse-E-3-E12, cDD-TE-4-E12, SI-4-E14-DMAPr, TL-1-12D-DMA, SY-010, and SY-011. The non-cationic lipid may be selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG (1,2-dioleoyl-sn-glycero-3-phospho-(1′-rac-glycerol)). The PEG-modified lipid may be DMG-PEG-2K.

An exemplary LNP in accordance with the invention may be composed of a cationic lipid selected from cKK-E12, cKK-E10, OF-Deg-Lin and OF-02; a non-cationic lipid selected from DOPE and DEPE; a cholesterol-based lipid such as cholesterol; and a PEG-modified lipid such as DMG-PEG-2K.

A further exemplary LNP in accordance with the invention may be composed of a cationic lipid selected from ICE, HGT4001, and HGT4002; a non-cationic lipid selected from DOPE and DEPE; and a PEG-modified lipid such as DMG-PEG-2K.

A further exemplary LNP in accordance with the invention may be composed of a cationic lipid selected from GL-TES-SA-DMP-E18-2 and GL-TES-SA-DME-E18-2; a non-cationic lipid selected from DOPE and DEPE; a cholesterol-based lipid such as cholesterol; and a PEG-modified lipid such as DMG-PEG-2K.

A further exemplary LNP in accordance with the invention may be composed of a cationic lipid selected from TL1-01D-DMA, TL1-04D-DMA, TL1-08D-DMA, and TL1-10D-DMA; a non-cationic lipid selected from DOPE and DEPE; a cholesterol-based lipid such as cholesterol; and a PEG-modified lipid such as DMG-PEG-2K.

A further exemplary LNP in accordance with the invention may be composed of a cationic lipid which is HEP-E3-E10 or HEP-E4-E10; a non-cationic lipid selected from DOPE and DEPE; a cholesterol-based lipid such as cholesterol; and a PEG-modified lipid such as DMG-PEG-2K.

A further exemplary LNP in accordance with the invention may be composed of a cationic lipid which is SY-3-E14-DMAPr; a non-cationic lipid selected from DOPE and DEPE; a cholesterol-based lipid such as cholesterol; and a PEG-modified lipid such as DMG-PEG-2K.

In some embodiments, the cationic lipid constitutes about 30-60% of the LNP by molar ratio. For example, the cationic lipid may constitute about 35-40%. In some embodiments, the ratio of cationic lipid to non-cationic lipid to cholesterol-based lipid to PEG-modified lipid in an LNP in accordance with the invention is approximately 30-60:25-35:20-30:1-15 by molar ratio. In other embodiments, the ratio of cationic lipid to non-cationic lipid to PEG-modified lipid in an LNP in accordance with the invention is approximately 55-65:30-40:1-15 by molar ratio.

A typical LNP for use with the invention may be composed of one of the following combinations of a cationic lipid, a non-cationic lipid, a PEG-modified lipid and optionally cholesterol:
cKK-E12, DOPE, cholesterol and DMG-PEG2K;
cKK-E10, DOPE, cholesterol and DMG-PEG2K;
OF-Deg-Lin, DOPE, cholesterol and DMG-PEG2K;
OF-02, DOPE, cholesterol and DMG-PEG2K;
TL1-01D-DMA, DOPE, cholesterol and DMG-PEG2K;
TL1-04D-DMA, DOPE, cholesterol and DMG-PEG2K;
TL1-08D-DMA, DOPE, cholesterol and DMG-PEG2K;
TL1-10D-DMA, DOPE, cholesterol and DMG-PEG2K;
ICE, DOPE and DMG-PEG2K;
HGT4001, DOPE and DMG-PEG2K;
HGT4002, DOPE and DMG-PEG2K;
SY-3-E14-DMAPr, DOPE, cholesterol and DMG-PEG2K;
RL3-DMA-07D, DOPE, cholesterol and DMG-PEG2K;
RL2-DMP-07D, DOPE, cholesterol and DMG-PEG2K;
cHse-E-3-E10, DOPE, cholesterol and DMG-PEG2K;
cHse-E-3-E12, DOPE, cholesterol and DMG-PEG2K;
cDD-TE-4-E12, DOPE, cholesterol and DMG-PEG2K.

In some embodiments, the LNP comprises no more than three distinct lipid components. In such embodiments, the three distinct lipid components usually are a cationic lipid, a non-cationic lipid and a PEG-modified lipid. The non-cationic lipid can be DOPE or DEPE. The PEG-modified lipid can be DMG-PEG2K. In a specific embodiment, the three distinct lipid components are HGT4002, DOPE and DMG-PEG2K. In an exemplary embodiment, HGT4002, DOPE and DMG-PEG2K are present in a molar ratio of approximately 60:35:5, respectively.

In some embodiments, the LNP comprises four distinct lipid components. In such embodiments, the four distinct lipid components usually are a cationic lipid, a non-cationic lipid, cholesterol and a PEG-modified lipid. The non-cationic lipid can be DOPE or DEPE. In a specific embodiment, the non-cationic lipid is DOPE. The PEG-modified lipid can be DMG-PEG2K. The molar ratio of cationic lipid to non-cationic lipid to cholesterol to PEG-modified lipid typically is between about 30-60:25-35:20-30:1-15, respectively.

The invention provides compositions comprising the LNPs of the invention. Such compositions can be pharmaceutical compositions. The LNPs in these compositions typically have an average size of less than 150 nm, e.g., less than 100 nm. In a specific embodiment, the LNPs have an average size of about 50-70 nm, e.g., about 55-65 nm. In certain embodiments, the mRNA at a concentration of between about 0.5 mg/mL to about 1.0 mg/mL. In some embodiments, the LNPs are suspended in an aqueous solution comprising trehalose, e.g., 10% (w/v) trehalose in water. In other embodiments, LNPs are suspended in an aqueous solution comprising sucrose, e.g., 10% (w/v) sucrose in water.

The invention also provides a dry powder formulation comprising a plurality of spray-dried particles comprising the LNPs of the invention, and one or more polymers. In some embodiments, the spray-dried particles are inhalable. In other embodiments, the spray-dried particles are nebulizable upon reconstitution.

The invention further provides a kit comprising a composition in accordance with the invention.

The mRNAs of the invention are for use in therapy. For example, the invention provides a method of treating or preventing an infection in a subject caused by a virus which uses human ACE2 protein for cellular entry, wherein the method comprises administering a composition in accordance with the invention to the subject. The invention also provides a method of neutralizing a virus which uses human ACE2 protein for cellular entry in a subject, wherein the method comprises administering a composition of the invention to the subject. In some embodiments, the virus is a coronavirus. In particular embodiments, the coronavirus is a beta-coronavirus. In a specific embodiment, the beta-coronavirus is SARS-CoV-2.

In specific embodiments, the subject has, or is diagnosed with, coronavirus disease (COVID-19). In some embodiments, the coronavirus disease (COVID-19) is mild coronavirus disease (COVID-19). In other embodiments, the coronavirus disease (COVID-19) is severe coronavirus disease (COVID-19). In some embodiments, the subject has a chronic infection. In some embodiments, the subject is immunocompromised or immunosuppressed. In some embodiments, the subject has not been vaccinated against COVID-19 or has not been vaccinated against SARS-CoV-2 infection. In some embodiments, the subject cannot be safely vaccinated against COVID-19 or against SARS-CoV-2 infection. In some embodiments, the subject is clinically vulnerable to COVID-19.

In some embodiments, the subject is 50 years of age and over, 55 years of age and over, 60 years of age and over, 65 years of age and over, 65 years of age and over, 70 years of age and over, 75 years of age and over, 80 years of age and over, 85 years of age and over, 90 years of age and over, or 95 years of age and over.

In some embodiments, the therapeutic methods described herein comprise administering a composition of the invention as an aerosol. In some embodiments, administration of the composition is carried out on an outpatient basis. The aerosol may be administered by intranasal administration. Alternatively, the aerosol may be administered by pulmonary administration. For example, the aerosol may be administered by nebulization.

In a specific embodiment, a composition of the invention is nebulized to generate nebulized particles containing the composition for inhalation by the subject. Nebulized particles for inhalation by a subject typically have an average size less than 8 μm. In some embodiments, the nebulized particles for inhalation by a subject have an average size between approximately 1-8 μm. In particular embodiments, the nebulized particles for inhalation by a subject have an average size between approximately 1-5 μm. In one specific embodiment, the fine particle fraction (FPF) of the nebulized composition with an average particle size less than 5 μm is at least about 30%, more typically at least about 40%, e.g., at least about 50%, more typically at least about 60%. In another specific embodiment, the mean respirable emitted dose (i.e., the percentage of FPF with a particle size <5 μm; e.g., as determined by next generation impactor with 15 L/min extraction) is at least about 30% of the emitted dose, e.g., at least about 31%, at least about 32%, at least about 33%, at least about 34%, or at least about 35% the emitted dose. In yet another specific embodiment, the mean respirable delivered dose (i.e., the percentage of FPF with a particle size <5 μm; e.g., as determined by next generation impactor with 15 L/min extraction) is at least about 15% of the emitted dose, e.g. at least 16% or 16.5% of the emitted dose.

In some embodiments, nebulization is performed with a nebulizer. In a specific embodiment, the reservoir volume of the nebulizer ranges from about 5.0 mL to about 8.0 mL. For example, the reservoir volume of the nebulizer may be about 5.0 mL, is about 6.0 mL, is about 7.0 mL, or is about 8.0 mL.

In some embodiments, at least about 60%, e.g., at least about 65% or at least about 70%, of the mRNA maintains its integrity after nebulization.

In some embodiments, the nebulization rate is greater than 0.2 mL/min, e.g. greater than 0.25 mL/min, greater than 0.3 mL/min, greater than 0.45 mL/min, or typically ranges between 0.2 mL/min and 0.5 mL/min.

In some embodiments, the therapeutic methods described herein comprise administering a composition of the invention intravenously.

In some embodiments, the therapeutic methods described herein comprise administering a composition of the invention once. In other embodiments, the composition is administered at least once. For example, the composition may be administered at least twice. Alternatively, the composition may be administered at least three times. The interval between administrations may be 1 day, 2 days, 3 days, 4 days, 5 days, 6 days, 7 days, or 8 days.

In some embodiments, the therapeutic methods described herein comprise administering a composition of the invention before and/or after exposure, or suspected exposure, to a virus which uses human ACE2 protein for cellular entry in a subject. For example, a composition of the invention may be administered to a subject after infection with such a virus. In some embodiments, a composition of the invention is administered to a subject after exposure, or suspected exposure, to a virus which uses human ACE2 protein for cellular entry in a subject (such as SARS-CoV-2), and before the onset of at least one symptom of coronavirus disease (COVID-19).

In a particular embodiment, the invention provides a method of treating or preventing an infection in a subject caused by a virus which uses human ACE2 protein for cellular entry, wherein the method comprises administering a composition comprising the mRNA of the invention encapsulated in a lipid nanoparticle (LNP) to the subject intravenously. In another particular embodiment, the invention provides a method of neutralizing a virus which uses human ACE2 protein for cellular entry in a subject, wherein the method comprises administering a composition comprising the mRNA of the invention encapsulated in a lipid nanoparticle (LNP) to the subject intravenously. In such embodiments, the polypeptide encoded by the mRNA of the invention usually comprises an immunoglobulin Fc region, typically linked to the C-terminus of the extracellular domain of human ACE2 protein or portion thereof.

The lipid component of LNP typically consists of a cationic lipid, a non-cationic lipid, a PEG-modified lipid, and optionally a cholesterol-based lipid. For example, the cationic lipid may be selected from cKK-E12, cKK-E10, OF-Deg-Lin, and OF-02. The cationic lipid also may be selected from RL3-DMA-07D, RL2-DMP-07D, cHse-E-3-E10, cHse-E-3-E12, and cDD-TE-4-E12. The non-cationic lipid may be selected from DOPE and DEPE. The cholesterol-based lipid may be cholesterol. The PEG-modified lipid may be DMG-PEG-2K.

In a further particular embodiment, the invention provides a method of treating or preventing an infection in a subject caused by a virus which uses human ACE2 protein for cellular entry, wherein the method comprises administering a composition comprising the mRNA of the invention encapsulated in a lipid nanoparticle (LNP) to the subject by nebulization. In yet a further particular embodiment, the invention provides a method of neutralizing a virus which uses human ACE2 protein for cellular entry in a subject, wherein the method comprises administering a composition comprising the mRNA of the invention encapsulated in a lipid nanoparticle (LNP) to the subject by nebulization. In such embodiments, the lipid component of lipid nanoparticle (LNP) typically consists of a cationic lipid, a non-cationic lipid, a PEG-modified lipid, and optionally cholesterol. For example, the cationic lipid may be selected from GL-TES-SA-DMP-E18-2, GL-TES-SA-DME-E18-2, TL1-01D-DMA, TL1-04D-DMA, SY-3-E14-DMAPr, TL1-10D-DMA, HEP-E3-E10, HEP-E4-E10, ICE, HGT4002, SI-4-E14-DMAPr, TL1-12D-DMA, SY-010, and SY-011. The non-cationic lipid may be selected from DOPE and DEPE. The PEG-modified lipid may be DMG-PEG-2K. A particularly suitable LNP may be composed of ICE and HGT4002 as the cationic lipid, DOPE as the non-cationic lipid is DOPE, and DMG-PEG-2K as the PEG-modified lipid.

### BRIEF DESCRIPTION OF THE DRAWINGS

Embodiments of the invention will be described, by way of example, with reference to the following drawings, in which:

FIG. 1 is a schematic illustration of a computational method that can generate optimized nucleotide sequences suitable for use with the present invention.

FIG. 2 is a schematic illustration of the domain structure of the human angiotensin-converting enzyme 2 (ACE2) protein.

FIG. 3 is a schematic illustration of the polypeptides encoded by mRNAs of the invention. These polypeptides comprise the extracellular domain of human ACE2 protein and may include (a) one or more amino acid substitutions relative to the naturally occurring human ACE2 protein to increase the binding affinity to the spike (S) glycoprotein of SARS-CoV-2 and/or (b) an immunoglobulin Fc region and an optional localization sequence.

FIG. 4A illustrates effective secretion of polypeptides comprising the extracellular domain of human ACE2 protein after transfection of human cells with the sequence-optimized mRNAs of the invention. The depicted western blots show the presence of the expressed polypeptides in cell lysates and the cell supernatants, detected using two primary antibodies. FIG. 4B is a bar graph depicting the quantification of the western blot data shown in FIG. 4A.

FIG. 5A illustrates in vivo expression of polypeptides comprising the extracellular domain of human ACE2 protein after lung delivery of the sequence-optimized mRNAs of the invention. The depicted western blot shows the presence of the expressed polypeptides in bronchoalveolar lavage fluid (BALF) samples from mice following intratracheal administration of lipid nanoparticles (LNPs) encapsulating the mRNAs. FIG. 5B is a bar graph depicting the quantification of the western blot data shown in FIG. 5A.

FIG. 6A and FIG. 6B illustrate in vivo expression of polypeptides comprising the extracellular domain of human ACE2 protein including one or more amino acid substitutions relative to a naturally occurring human ACE2 protein after lung delivery of the sequence-optimized mRNAs of the invention. The depicted western blots show the presence of the expressed polypeptides in BALF samples from mice following intratracheal administration of LNPs encapsulating the mRNAs. FIG. 6C is a bar graph depicting the quantification of the western blot data shown in FIGS. 6A and 6B.

FIG. 7A illustrates prolonged in vivo expression of polypeptides comprising the extracellular domain of human ACE2 protein after lung delivery of the sequence-optimized mRNAs of the invention. The depicted western blots show the presence of the expressed polypeptides in BALF samples from mice taken at various time points after intratracheal administration of LNPs encapsulating the mRNAs. FIG. 7B is a line graph depicting the quantification of the western blot data shown in FIG. 7A.

FIG. 8 illustrates effective secretion of polypeptides comprising the extracellular domain of human ACE2 protein and an immunoglobulin Fc region after transfection of human cells with mRNAs of the invention. The depicted western blot shows the presence of the expressed polypeptides in cell supernatants.

FIG. 9A illustrates schematically a Human Bronchial Epithelial Cell-Air Liquid Interface (HBEC-ALI) culture and a timeline for establishing it. FIG. 9B is an example micrograph of the differentiated epithelium formed in an established HBEC-ALI culture. The sample was stained with hematoxylin and eosin (H&E).

FIGS. 10A and 10B illustrate successful transcytosis of polypeptides comprising the extracellular domain of human ACE2 protein and an immunoglobulin Fc region across the differentiated epithelium to the apical side of an HBEC-ALI culture after the addition of conditioned media from HEK293 cells transfected with mRNAs of the invention to the basolateral growth medium of the HBEC-ALI culture. The depicted western blots show the presence of the polypeptides in the conditioned media (Input), the mucus wash samples taken from the apical surface of HBEC-ALI cultures incubated with conditioned media containing the polypeptides encoded by the mRNA of the invention alone (Sup), and the mucus wash samples taken from the apical surface of HBEC-ALI cultures incubated with conditioned media mixed with HBEC-ALI culture medium in a 1:1 ratio (Mix). The white arrows point to the bands in the mucus wash samples representing the transcytosed polypeptides.

FIGS. 11A and 11B illustrate the prolonged presence of polypeptides comprising the extracellular domain of human ACE2 protein and an immunoglobulin Fc region in the lungs of mice after intravenous administration of mRNAs of the invention. The depicted western blots shows the presence of the expressed polypeptides in BALF samples from mice taken at various time points after intravenous administration of LNPs encapsulating the mRNAs. FIG. 11C is a bar graph depicting the quantification of the western blot data shown in FIGS. 11A and 11B. FIG. 11D is a bar graph depicting the quantification of western blot data showing the presence of the polypeptides in the serum of mice at the indicated time points after intravenous administration of LNPs. The LNPs of FIGS. 11A-D comprised cKK-E12, DOPE, cholesterol and DMG-PEG2K at molar ratios of 40:30:35:5. Panels E and F of FIG. 11 shows a series of exemplary graphs illustrating in vivo expression of various soluble ACE2 polypeptides of the present invention in BALF and serum, respectively, after intravenous administration, as measured by ELISA. FIG. 11G shows an exemplary Western blot comparing expression of soluble ACE2 polypeptide encoded by SEQ ID NO: 3 in BALF fluid and serum after intravenous dosing. FIG. 11H shows dimerization of sACE2 and sACE2-Fc in unreduced mouse BALF. Western blots of the constructs expressed by SEQ ID NO: 2 and SEQ ID NO: 3 in BALF of CD-1 mice 24 hours after intravenous dosing with the LNPs. The LNPs of FIGS. 11-E-H comprised HGT4002, DOPE, and DMG-PEG2K at molar ratios of 60:35:5.

FIG. 12 illustrates in vivo expression of a polypeptide comprising the extracellular domain of human ACE2 protein and an immunoglobulin Fc region after lung delivery of a sequence-optimized mRNA of the invention. FIG. 12 is a bar graph depicting ELISA data showing the presence of the expressed polypeptide encoded by SEQ ID NO: 3 in BALF samples taken from CD-1 mice and Syrian hamsters, respectively.

FIG. 13 illustrates the effective neutralization of pseudovirus particles displaying the spike (S) glycoprotein of SARS-CoV-2 in vitro by polypeptides comprising the extracellular domain of human ACE2 protein. Cultured human cells were transfected with mRNAs of the invention, resulting in the effective secretion of the mRNA-encoded polypeptides into cell culture supernatant, as illustrated in FIG. 13A. These supernatants were applied to a reporter virus particle (RVP) neutralization assay. The RVPs displayed the spike (S) glycoprotein of SARS-CoV-2. Panels B-D of FIG. 13 show line graphs depicting the percentage of cells infected with RVPs displaying the SARS-CoV-2 spike protein of the Wuhan variant, the D614G variant, and the South African variant, respectively, after incubation with the supernatants diluted as indicated.

FIG. 14 illustrates the effective neutralization of pseudovirus particles displaying the spike (S) glycoprotein of SARS-CoV-2 in vitro by polypeptides comprising the extracellular domain of human ACE2 protein. FIG. 14A shows a line graph depicting the percentage of cells infected with UK S glycoprotein variant RVPs after incubation with the variant human ACE2 polypeptides encoded by SEQ ID Nos: 2, 4, 5 and 6, respectively. RVPs incubated with medium only served as a baseline. at the indicated dilutions. FIG. 14B shows a line graph depicting the relative percentage cells infected with delta S glycoprotein variant RVPs after incubation with the variant human ACE2 polypeptides encoded by SEQ ID Nos: 2, 4, 5 and 6, respectively, at concentrations as indicated.

FIG. 15 illustrates the effective neutralization of pseudovirus particles displaying different spike (S) glycoprotein variants of SARS-CoV-2 in vitro by a polypeptide comprising the extracellular domain of human ACE2 protein and an immunoglobulin Fc region (sACE2-Fc fusion protein). It shows an exemplary line graph depicting the percentage of cells infected with Wuhan, UK, SA, Brazil, California, and Ohio S glycoprotein variant RVPs, and a dilution matched control. It illustrates potent neutralization from the sACE2-Fc fusion protein encoded by SEQ ID NO: 3.

FIG. 16 illustrates the effective neutralization of pseudovirus particles displaying the spike (S) glycoprotein of SARS-CoV-2 in vitro by a polypeptide comprising the extracellular domain of human ACE2 protein and an immunoglobulin Fc region (sACE2-Fc fusion protein). It shows an exemplary line graph depicting the percentage of cells infected with Wuhan-Hu-1, Alpha (UK), Beta (South African), Gamma (Brazil), Delta (India), Eta (UK) and Iota (New York) S glycoprotein variant RVPs, and a dilution matched control. It illustrates potent neutralization from the sACE2-Fc fusion protein encoded by SEQ ID NO: 3.

FIG. 17 illustrates the neutralization of SARS-CoV-2 in a Syrian Golden hamster challenge study, following intratracheal administration of LNPs loaded with sequence-optimized mRNAs of the invention encoding sACE2 (SEQ ID NO: 2) or sACE2-Fc (SEQ ID NO: 3). The LNPs comprises SY-3-E14-DMAPr, DOPE, cholesterol and DMG-PEG2K at molar ratios of 50:15:30:5. FIG. 17A shows a line graph of percent bodyweight change over time in hamsters following the administration of the LNPs and subsequent challenge with SARS-CoV-2. The viral challenge occurred 1 day after LNP administration. Saline treated hamsters with and without infection served as controls.

FIG. 17B shows a bar graph of viral load in hamsters sacrificed on day 3 after infection with SARS-CoV-2. It demonstrates decreased viral load in hamsters pre-treated with LNPs encapsulating a sequence-optimized sACE2-encoding mRNA of the invention. Statistical analysis for bodyweight was an unpaired two-tailed t-test, and statistical analysis for TCID50 was an unpaired two-tailed t-test with Welch's adjustment. * represents p≤0.05, ** represents p≤0.01, *** represents p≤0.001, and **** represents p≤0.0001.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

As used herein, the term “extracellular domain of human ACE2 protein” may refer to the entire extracellular domain, or a portion thereof, which retains the ability to bind to a viral surface protein. In some embodiments, the term refers to amino acid residues 1-740 of the naturally occurring human ACE2 protein.

As used herein, the term “mRNA” refers to a polyribonucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, in vitro transcribed, or chemically synthesized. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a poly(A) tail.

As used herein the term “sequence-optimized” is used to describe a nucleotide sequence that is modified relative to a naturally-occurring or wild-type nucleic acid. Such modifications may include, e.g., codon optimization as well as the use of 5′ UTRs and 3′ UTRs which are not normally associated with the naturally-occurring or wild-type nucleic acid. As used herein, the terms “codon optimization” and “codon-optimized” refer to modifications of the codon composition of a naturally-occurring or wild-type nucleic acid encoding a peptide, polypeptide or protein that do not alter its amino acid sequence, thereby improving protein expression of said nucleic acid. In the context of the present invention, “codon optimization” may also refer to the process by which one or more optimized nucleotide sequences are arrived at by removing with filters less than optimal nucleotide sequences from a list of nucleotide sequences, such as filtering by guanine-cytosine content, codon adaptation index, presence of destabilizing nucleic acid sequences or motifs, and/or presence of pause sites and/or terminator signals.

As used herein, the term “template DNA” (or “DNA template”) relates to a DNA molecule comprising a nucleic acid sequence encoding an mRNA transcript to be synthesized by in vitro transcription. The template DNA is used as template for in vitro transcription in order to produce the mRNA transcript encoded by the template DNA. The template DNA comprises all elements necessary for in vitro transcription, particularly a promoter element for binding of a DNA-dependent RNA polymerase, such as, e.g., T3, T7 and SP6 RNA polymerases, which is operably linked to the DNA sequence encoding a desired mRNA transcript. Furthermore the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

As used herein, the term “decoy receptor” relates to a version of a cellular receptor which cannot be used by a pathogen for cellular entry, wherein the cellular receptor is normally present on the surface of a cell and used by a pathogen for entry into the cell. For example, the pathogen may be a bacterium or a virus. The decoy receptor may lack one or more transmembrane regions of the receptor which normally tether the receptor to the cell membrane. The decoy receptor may be secreted by a cell. The decoy receptor may also be translocated across a cell or layer of cells.

As used herein, the term “localization sequence” relates to an amino acid sequence which facilitates transcytosis of a linked polypeptide across an epithelium. The localization sequence may be linked to the carboxy-terminus (C-terminus) of a polypeptide. The localization sequence may be linked to the polypeptide by a linker sequence. The localization sequence may also be exogenous to the polypeptide. For example, the localization sequence may facilitate transport of the linked polypeptide across a layer of airway epithelial cells such that polypeptides including one of these sequences may be more effectively delivered to the airway or lung lumen.

As used herein, the term “subject” refers to a mammal, such as a human or other animal. Typically, a subject is a human. The subject can be male or female and can be any suitable age, including infant, juvenile, adolescent, adult, and geriatric subjects.

As used herein, the term “N/P ratio” refers to a molar ratio of cationic lipids in a lipid nanoparticle relative to mRNA encapsulated within that lipid nanoparticle. As such, N/P ratio is typically calculated as the ratio of moles of cationic lipids in a lipid nanoparticle relative to moles of mRNA encapsulated within that nanoparticle. For example, a 4-fold molar excess of cationic lipid per mole mRNA is referred to as an “N/P” ratio of 4.

All technical and scientific terms used herein have the same meaning as commonly understood by one of ordinary skill in the art to which this application belongs and as commonly used in the art to which this application belongs. The publications and other reference materials referenced herein to describe the background of the invention and to provide additional detail regarding its practice are hereby incorporated by reference.

### DETAILED DESCRIPTION OF THE INVENTION

mRNA has been shown to be an effective agent for the delivery of vaccine antigens to subjects. The use of mRNA has the advantage that it can be rapidly synthesized at scale to find utility in pandemic situations caused by a rapidly spreading infectious agent, such as a viral pathogen. However, antigen-based vaccination approaches have the disadvantage that they may become ineffective against a viral pathogen due to randomly occurring genetic modifications and the selective pressure such pathogens are exposed to during their life cycle. Moreover, vaccine-based approaches are useful to prevent future infections, but typically do not protect subjects that have already been exposed to the viral pathogen and may begin to show signs of disease. Therefore, alternative approaches are needed for limiting the spread of infection in subjects that have been exposed to an easily transmissible viral pathogen. Ideally, such alternative approaches are designed to take advantage of the rapid deployment afforded by mRNA therapeutics.

Over the last two decades, viruses which use the human angiotensin-converting enzyme 2 (ACE2) protein for cellular entry have caused several fast-spreading epidemics or pandemics in the human population. The present invention provides compositions that are useful in treating or preventing an infection in a subject caused by such viruses. The compositions of the invention act by neutralizing such viruses, preventing them from binding to the human ACE2 protein and gaining cellular entry. In particular, the invention provides an mRNA comprising an optimized nucleotide sequence encoding a polypeptide comprising the extracellular domain of ACE2 protein, or a portion thereof, which binds to a viral surface protein. The mRNA may be encapsulated in lipid nanoparticles (LNPs) for delivery to a subject in vivo. Typically, compositions comprising the LNPs are delivered to a subject either intravenously or as an aerosol, for example via nebulization.

Viruses which Use the Human ACE2 Protein for Cellular Entry

### Coronaviruses

Coronaviruses (CoVs) are the largest group of viruses belonging to the Nidovirales order, which includes Coronaviridae, Arteriviridae, and Roniviridae families. CoVs are spherical enveloped viruses with a positive-sense single-stranded RNA genome and a nucleocapsid of helical symmetry with a diameter of approximately 125 nm.

### SARS-CoV and SARS-CoV-2.

Viruses which use the human ACE2 protein as a cell surface receptor for cellular entry have caused at least one widespread epidemic involving severe acute respiratory syndrome (SARS), and one ongoing global pandemic involving Coronavirus Disease 2019 (COVID-19). The causative agent of SARS is SARS coronavirus (SARS-CoV), and the causative agent of COVID-19 is SARS coronavirus 2 (SARS-CoV-2). SARS-CoV-2 is a beta-coronavirus. Infections caused by viruses, which rely on binding to the angiotensin-converting enzyme (ACE2) receptor to enter cells of a subject, can cause acute pneumonia, respiratory distress, and other organ damage in subjects and are associated with high mortality, in particular in subjects above 50 years of age.

Subjects can be infected with SARS-CoV and SARS-CoV-2 through direct contact with contaminated surfaces, direct contact with respiratory droplets of an infected person, and in particular, through airborne transmission. SARS-CoV and SARS-CoV-2 frequently establish infection through binding to the ACE2 receptors present on mucosal and airway epithelial cells of subjects, entering mucosal and airway epithelial cells, and commencing viral replication.

SARS-CoV-2 spike (S) Glycoprotein

Cellular entry of SARS-CoV-2 depends on the binding of spike (S) glycoproteins to receptors on the cell surface and on S protein priming by host cell proteases. The S protein comprises two functional subunits responsible for binding to the host cell receptor (S1 subunit) and fusion of the viral and cellular membranes (S2 subunit). The S protein forms a homotrimer that produces a distinctive spike structure on the surface of the virus. The S1 subunit has a large receptor-binding domain (RBD), while S2 forms the stalk of the spike molecule. The RBD of the SARS-CoV-2 spike (S) glycoprotein, in homotrimeric form, binds to the human ACE2 protein receptor on cell surfaces. A serine protease present on the cell surface, TMPRS22, primes the spike (S) glycoprotein for fusion with the cell membrane in the process of cellular entry. The amino acid sequence of the full-length SARS-CoV-2 S glycoprotein can be found at GenBank accession no. QHD43416.1. The S1 subunit is located at residues 1 to 681, the S2 subunit is located at residues 686 to 1208 and the S2′ subunit is located at residues 816 to 1208. The C-terminal end of the S protein contains a transmembrane domain, and the last 19 amino acids of the cytoplasmic tail contain an endoplasmic reticulum (ER)-retention signal.

### Variants of SARS-CoV-2 and Mutations in Spike (S) Glycoprotein

Although the observed diversity among pandemic SARS-CoV-2 sequences is low, its rapid global spread provides the virus with ample opportunity for natural selection to act upon rare but favorable mutations. It is advantageous to target the circulating SARS-CoV-2 virus variants using polypeptides comprising the extracellular domain of human angiotensin-converting enzyme 2 (ACE2) protein, or a portion thereof, rather than just targeting the index strain from Wuhan. The soluble ACE2 ‘decoy receptors’ encoded by the mRNAs provided herein provide robust protection against SARS-CoV-2, despite the presence of mutations in the viral spike (S) glycoprotein which may facilitate immune evasion, provided the virus still relies on the ACE2 receptor for cellular entry.

An amino acid change in the SARS-CoV-2 S glycoprotein, D614G, emerged early during the 2020 COVID-19 pandemic and as of March 2021 may be the most prevalent form of the virus around the world. Patients infected with G614 shed more viral nucleic acid compared with those with D614, and G614-bearing viruses show significantly higher infectious titers in vitro than their D614 counterparts (Korber et al., 2020, Cell 182, 1-16).

Other rare mutations that have been identified in the SARS-CoV-2 S protein are summarized in the table below (Korber et al. 2020, https://doi.org/10.1101/2020.04.29.069054):

| Spike Protein Mutation | Spike location possible impact |
|---|---|
| L5F | Signal Peptide |
| L8V/W | Signal Peptide |
| H49Y | S1 NTD domain |
| Y145H/del | S1 NTD domain |
| Q239K | S1 NTD domain |
| V367F | Up/Down conformations |
| G476S | Directly in the RBD |
| V483A | Up/Down conformations |
| V6151/F | In SARS-CoV ADE epitope |
| A831V | Potential fusion peptide in S2 |
| D839Y/N/E | S2 subunit |
| S943P | Fusion core of HR1 |
| P1263L | Cytoplasmic Tail |

Further SARS-CoV-2 S glycoprotein mutations include: L18F, HV 69-70 deletion (Δ69-70), Y144 deletion (ΔY144), E154Q, Q218E, A222V, S447N, F490S, S494P, N501Y, A570D, E583D, T618E, P681H, A701V, T716I, T7231, 1843V, S982A and D1118H. In late 2020, new SARS-CoV-2 variants emerged in the UK, South Africa, Brazil and California that contained multiple mutations. The mutations present in the SARS-CoV-2 S glycoprotein in the UK variant (named lineage B.1.1.7) include a H69 deletion (ΔH69), V70 deletion (ΔV70), a Y144 deletion (ΔY144), N501Y, A570D, P681H, T716I, S982A and D1118H mutations (Rambaut et al. 2020 https://virological/org/t/preliminary-genomic-characterisation-of-an-emergent-sars-cov-2-lineage-in-the-uk-defined-by-a-novel-set-of-spike-mutatations/563). In October 2020, the South African variant (named lineage B.1.351) emerged, which includes six mutations in the SARS-CoV-2 S glycoprotein protein—D80A, K417N, E484K, N501Y, D614G and A701V. By the end of November 2020, three further SARS-CoV-2 S glycoprotein mutations had emerged (L18F, R246I and K417N) and the deletion of three amino acids at L242 (ΔL242), A243 (ΔA243) and L244 (ΔL244) (Tegally et al. (2020) https://doi.org/10.1101/2020.12.21.20248640). The mutations present in the SARS-CoV-2 S glycoprotein in the Brazilian variant (named lineage P.1) include L18F, T20N, P26S, D138Y, R190S, K417T, E484K, N501Y, H655Y, T10271 and V1176F. The mutations present in the SARS-CoV-2 S glycoprotein in the Californian variant (known as CAL.20C) include S13I, W152C and L452R (Zhang et al. (2021) https://doi.org/1031101/2021.01.18.21249786).

### COVID-19

Coronavirus Disease 2019 (COVID-19) is an infectious disease caused by severe respiratory syndrome coronavirus 2 (SARS-CoV-2). The compositions of the invention (e.g., the optimized mRNAs described herein) are generally useful to treat subjects suffering from COVID-19. They can also be used as preventive therapy to avoid the development of COVID-19 in subjects who have been exposed to a patient suffering from COVID-19.

Subjects that may benefit from administration of the compositions of the invention (e.g., the optimized mRNAs described herein) may suffer from various forms of COVID-19. Subjects with mild COVID-19 may have symptoms including (a) a high temperature or fever, (b) a new, continuous cough, and/or (c) a loss of or change to the subject's sense of smell or taste. Severe COVID-19 may comprise symptoms including acute pneumonia, respiratory distress, or other organ damage. The compositions of the invention may be particularly beneficial to subjects suffering from severe COVID-19, many of whom may require hospitalization. Subjects with severe COVID-19 may (a) have hypoxia, (b) have oxygen saturation levels (SpO2) less than or equal to 92% as measured by pulse oximetry, (c) require non-invasive respiratory support, (d) require invasive respiratory support, or (e) require continuous positive airway pressure (CPAP) therapy.

Particular groups of subjects may be deemed clinically vulnerable to COVID-19. Prevention of COVID-19 post exposure may be the primary treatment goal in these subjects. Subjects clinically vulnerable to COVID-19 include subjects with one or more of the following conditions: (1) solid organ transplant recipients, (2) subjects with cancer who are undergoing chemotherapy, (3) subjects with lung cancer who are undergoing radiotherapy, (4) subjects with cancers of the blood or bone marrow such as leukaemia, lymphoma or myeloma, (5) subjects receiving immunotherapy or antibody treatments for cancer, (6) subjects having cancer treatments that can affect the immune system, such as protein kinase inhibitors or PARP inhibitors, (7) subjects who have had bone marrow or stem cell transplants in the last 6 months and/or who are taking immunosuppression drugs, (8) subjects with severe respiratory conditions including, e.g., cystic fibrosis, asthma and chronic obstructive pulmonary disease (COPD), (9) subjects with rare diseases that increase the risk of infections (such as severe combined immunodeficiency (SCID) or homozygous sickle cell disease), (10) subjects on immunosuppression therapies, (11) subjects with problems with their spleen, for example subjects who have had a splenectomy, (12) adult subjects with Down's syndrome, (13) adult subjects on dialysis or with chronic kidney disease (stage 5), (14) women who are pregnant with significant heart disease (congenital or acquired), and (15) other subjects who have been classed as clinically vulnerable, based on clinical judgement and an assessment of their needs.

### Decoy ACE2 Receptors

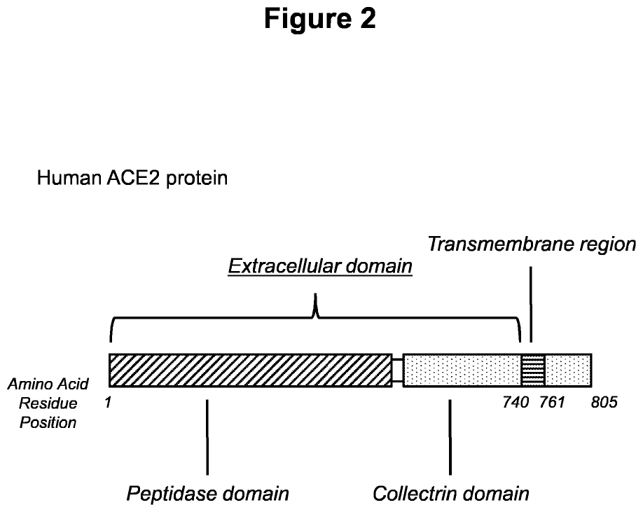

The invention provides an mRNA comprising an optimized nucleotide sequence encoding a polypeptide comprising the extracellular domain of human angiotensin-converting enzyme 2 (ACE2) protein, or a portion thereof, which binds to a viral surface protein. In a particular embodiment, the extracellular domain of human ACE2 protein comprises amino acid residues 1-740 of the naturally occurring human ACE2 protein encoded by SEQ ID NO: 10. To function as ‘decoy receptors’, the polypeptides of the invention do not comprise the transmembrane portion of the naturally occurring human ACE2 protein as illustrated in FIG. 2, to ensure that the viruses cannot use the polypeptides of the invention to enter and infect the cells of a subject.

The polypeptides described herein comprising the extracellular domain of human ACE2 protein can bind to the homotrimeric form of the spike (S) glycoprotein in the pre-fusion conformation present on the surface of SARS-CoV-2, thereby preventing the virus from utilizing this glycoprotein for cellular entry and neutralizing said virus. Typically, the polypeptides encoded by the mRNAs of the invention bind to a viral surface protein with a comparable binding affinity to the endogenous human ACE2 protein. In some embodiments, the polypeptides have improved binding affinity to the viral surface protein relative to the endogenous human ACE2 protein. Accordingly, the polypeptides of the invention may be able to ‘titrate’ away infectious viral particles and reduce the number of exposed viral surface proteins available to bind endogenous human ACE2 protein receptors present on the cell surface, or out-compete the endogenous human ACE2 protein receptors present on the cell surface for binding to the viral surface protein, thereby neutralizing the virus and/or preventing further infection of susceptible cells. Polypeptides with improved binding affinity may be generated by substituting one or more amino acid sequences in the extracellular domain of the naturally occurring SARS-CoV-2 protein, as explained in more detail below.

The soluble ACE2 ‘decoy receptors’ encoded by the mRNAs provided herein provide robust protection against SARS-CoV-2, despite the presence of mutations in the viral spike (S) glycoprotein which may facilitate immune evasion, provided the virus still relies on the ACE2 receptor for cellular entry. The ACE2 ‘decoy receptors’ encoded by the mRNAs provided herein may also be effective in neutralizing known or emerging viruses (e.g., SARS-CoV and other beta-coronaviruses which are able to infect animals and/or humans) which rely on the ACE2 receptor for cellular entry, in the future.

The binding between a polypeptide comprising the extracellular domain of human angiotensin-converting enzyme 2 (ACE2) protein and a viral surface protein can be determined using a range of well-established methods, for example: pull-down assays; immunoprecipitation assays; enzyme-linked immunosorbent assays (ELISA); flow cytometry assays; electrophoretic mobility shift assays (EMSA); labelled ligand-binding assays including fluorescent ligand binding assays, fluorescence resonance energy transfer (FRET) assays, surface plasmon resonance (SPR) assays; label-free ligand binding assays including biolayer inferometry (BLI) assays, nuclear magnetic resonance (NMR) assays, thermodynamic binding assays including isothermal titration calorimetry (ITC) assays; bead binding assays; and competition binding assays. Binding activity can be assessed relative to a reference polypeptide, such as the naturally occurring human ACE2 protein encoded by the amino acid sequence of SEQ ID NO: 10, or a polypeptide comprising the extracellular domain of human ACE2 protein, or a portion thereof, which binds a viral surface protein of interest. In a particular embodiment, the binding between a polypeptide comprising the extracellular domain of human ACE2 protein and a viral surface protein (e.g., the SARS-CoV-2 spike (S) glycoprotein) can be determined with a pseudovirus neutralization assay, e.g. the neutralization assay using Reporter Virus Particles (RVPs) as described in the examples. A polypeptide which is determined by such an assay to be more effective than a reference polypeptide in neutralizing pseudovirus particles carrying the viral surface protein of interest (e.g., the SARS-CoV-2 spike (S) glycoprotein) may have increased binding affinity.

### Amino Acid Substitutions

In specific embodiments, the extracellular domain of human ACE2 protein encoded by an mRNA of the invention contains one or more amino acid substitutions, relative to a naturally occurring human ACE2 protein encoded by the amino acid sequence of SEQ ID NO: 10. In typical embodiments, the one or more amino acid substitutions increase the binding affinity for SARS-CoV-2 spike (S) glycoprotein relative to a naturally occurring human ACE2 protein (e.g., as determined by a pseudovirus neutralization assay).

The ACE2 protein plays a role in the renin-angiotensin system (RAS) involved in the progression of cardiovascular diseases, such as hypertension, myocardial infarction, and heart failure. ACE2 converts the angiotensin II peptide to form angiotensin which promotes vasoconstriction and increases blood pressure, among other functions. The catalytic site of the human ACE2 protein is located in its extracellular domain and catalytic activity may require glycosylation of the protein. In accordance with the invention, polypeptides comprising the extracellular domain of human angiotensin-converting enzyme 2 (ACE2) protein are expressed at high levels in vivo. Accordingly, it may be advantageous to express catalytically inactive forms of the extracellular domain of human ACE2 to function as ‘decoy receptors’, to avoid undesired side effects. Accordingly, in some embodiments, the one or more amino acid substitutions inactivate the catalytic activity of human ACE2 protein. In other embodiments, the one or more amino acid substitutions affect the glycosylation of human ACE2 protein.

Amino acid substitutions that increase the binding affinity of human ACE2 protein for SARS-CoV-2 spike (S) glycoprotein, inactive the catalytic activity and/or affect is glycosylation are known in the art. For example, amino acid substitutions at position 273 of the human ACE2 protein have been reported to abolish catalytic activity (Ameratunga et al., N Z Med J. 2020 May 22; 133(1515):112-118). Amino acid substitutions at positions 90 and 92 of the human ACE2 protein have been reported to reduce N-glycosylation and increase the binding affinity of the ACE2 protein to the receptor binding domain of the spike (S) glycoprotein of SARS-CoV-2 (Linsky et al., Science. 2020 Dec. 4; 370(6521): pp. 1208-1214). Amino acid substitutions at positions 374 and 378 of the human ACE2 protein have been reported to abolish catalytic activity and improve binding affinity to the spike (S) glycoprotein of SARS-CoV-2 (Glasgow et al., Proc Natl Acad Sci USA. 2020 Nov. 10; 117(45): pp. 28046-28055).

Accordingly, in some embodiments, the one or more amino acid substitutions is at any one of the following amino acid positions of the naturally occurring human ACE2 protein encoded by the amino acid sequence of SEQ ID NO: 10: positions 90, 92, 273, 374, and 378. In particular embodiments, the one or more amino acid substitutions are selected from the group consisting of: N90D, T92A, R273A, H374N, and H378N. In one specific embodiment, the one or more amino acid substitutions comprise or are N90D and R273A. In another specific embodiment, the one or more amino acid substitutions comprise or are N90D and T92A. In yet another specific embodiment, the one or more amino acid substitutions comprise or are H374N and H378N.

In some embodiments, the amino acid sequence of the polypeptide encoded by an mRNA of the invention is at least 90%, 95%, 96%, 97%, 98%, or 99% identical to the amino acid sequence of any one of SEQ ID NOs: 11 to 18, 34 and 35. In particular embodiments, the amino acid sequence of the polypeptide is at least 90% identical to the amino acid sequence of any one of SEQ ID NOs: 11 to 18, 34 and 35. In particular embodiments, the amino acid sequence of the polypeptide is at least 95% identical to the amino acid sequence of any one of SEQ ID NOs: 11 to 18, 34 and 35. In particular embodiments, the amino acid sequence of the polypeptide is at least 96% identical to the amino acid sequence of any one of SEQ ID NOs: 11 to 18, 34 and 35. In particular embodiments, the amino acid sequence of the polypeptide is at least 97% identical to the amino acid sequence of any one of SEQ ID NOs: 11 to 18, 34 and 35. In particular embodiments, the amino acid sequence of the polypeptide is at least 98% identical to the amino acid sequence of any one of SEQ ID NOs: 11 to 18, 34 and 35. In particular embodiments, the amino acid sequence of the polypeptide is at least 99% identical to the amino acid sequence of any one of SEQ ID NOs: 11 to 18, 34 and 35.

In a specific embodiment, the amino acid sequence of the polypeptide is at least 90% identical to the amino acid sequence of SEQ ID NO: 11. In another specific embodiment, the amino acid sequence of the polypeptide is at least 95% identical to the amino acid sequence of SEQ ID NO: 11. In another specific embodiment, the amino acid sequence of the polypeptide is at least 96% identical to the amino acid sequence of SEQ ID NO: 11. In another specific embodiment, the amino acid sequence of the polypeptide is at least 97% identical to the amino acid sequence of SEQ ID NO: 11. In another specific embodiment, the amino acid sequence of the polypeptide is at least 98% identical to the amino acid sequence of SEQ ID NO: 11. In another specific embodiment, the amino acid sequence of the polypeptide is at least 99% identical to the amino acid sequence of SEQ ID NO: 11.

In a specific embodiment, the amino acid sequence of the polypeptide is at least 90% identical to the amino acid sequence of SEQ ID NO: 12. In another specific embodiment, the amino acid sequence of the polypeptide is at least 95% identical to the amino acid sequence of SEQ ID NO: 12. In another specific embodiment, the amino acid sequence of the polypeptide is at least 96% identical to the amino acid sequence of SEQ ID NO: 12. In another specific embodiment, the amino acid sequence of the polypeptide is at least 97% identical to the amino acid sequence of SEQ ID NO: 12. In another specific embodiment, the amino acid sequence of the polypeptide is at least 98% identical to the amino acid sequence of SEQ ID NO: 12. In another specific embodiment, the amino acid sequence of the polypeptide is at least 99% identical to the amino acid sequence of SEQ ID NO: 12.

In a specific embodiment, the amino acid sequence of the polypeptide is at least 90% identical to the amino acid sequence of SEQ ID NO: 13. In another specific embodiment, the amino acid sequence of the polypeptide is at least 95% identical to the amino acid sequence of SEQ ID NO: 13. In another specific embodiment, the amino acid sequence of the polypeptide is at least 96% identical to the amino acid sequence of SEQ ID NO: 13. In another specific embodiment, the amino acid sequence of the polypeptide is at least 97% identical to the amino acid sequence of SEQ ID NO: 13. In another specific embodiment, the amino acid sequence of the polypeptide is at least 98% identical to the amino acid sequence of SEQ ID NO: 13. In another specific embodiment, the amino acid sequence of the polypeptide is at least 99% identical to the amino acid sequence of SEQ ID NO: 13.

In a specific embodiment, the amino acid sequence of the polypeptide is at least 90% identical to the amino acid sequence of SEQ ID NO: 14. In another specific embodiment, the amino acid sequence of the polypeptide is at least 95% identical to the amino acid sequence of SEQ ID NO: 14. In another specific embodiment, the amino acid sequence of the polypeptide is at least 96% identical to the amino acid sequence of SEQ ID NO: 14. In another specific embodiment, the amino acid sequence of the polypeptide is at least 97% identical to the amino acid sequence of SEQ ID NO: 14. In another specific embodiment, the amino acid sequence of the polypeptide is at least 98% identical to the amino acid sequence of SEQ ID NO: 14. In another specific embodiment, the amino acid sequence of the polypeptide is at least 99% identical to the amino acid sequence of SEQ ID NO: 14.

In a specific embodiment, the amino acid sequence of the polypeptide is at least 90% identical to the amino acid sequence of SEQ ID NO: 15. In another specific embodiment, the amino acid sequence of the polypeptide is at least 95% identical to the amino acid sequence of SEQ ID NO: 15. In another specific embodiment, the amino acid sequence of the polypeptide is at least 96% identical to the amino acid sequence of SEQ ID NO: 15. In another specific embodiment, the amino acid sequence of the polypeptide is at least 97% identical to the amino acid sequence of SEQ ID NO: 15. In another specific embodiment, the amino acid sequence of the polypeptide is at least 98% identical to the amino acid sequence of SEQ ID NO: 15. In another specific embodiment, the amino acid sequence of the polypeptide is at least 99% identical to the amino acid sequence of SEQ ID NO: 15.

In a specific embodiment, the amino acid sequence of the polypeptide is at least 90% identical to the amino acid sequence of SEQ ID NO: 16. In another specific embodiment, the amino acid sequence of the polypeptide is at least 95% identical to the amino acid sequence of SEQ ID NO: 16. In another specific embodiment, the amino acid sequence of the polypeptide is at least 96% identical to the amino acid sequence of SEQ ID NO: 16. In another specific embodiment, the amino acid sequence of the polypeptide is at least 97% identical to the amino acid sequence of SEQ ID NO: 16. In another specific embodiment, the amino acid sequence of the polypeptide is at least 98% identical to the amino acid sequence of SEQ ID NO: 16. In another specific embodiment, the amino acid sequence of the polypeptide is at least 99% identical to the amino acid sequence of SEQ ID NO: 16.

In a specific embodiment, the amino acid sequence of the polypeptide is at least 90% identical to the amino acid sequence of SEQ ID NO: 17. In another specific embodiment, the amino acid sequence of the polypeptide is at least 95% identical to the amino acid sequence of SEQ ID NO: 17. In another specific embodiment, the amino acid sequence of the polypeptide is at least 96% identical to the amino acid sequence of SEQ ID NO: 17. In another specific embodiment, the amino acid sequence of the polypeptide is at least 97% identical to the amino acid sequence of SEQ ID NO: 17. In another specific embodiment, the amino acid sequence of the polypeptide is at least 98% identical to the amino acid sequence of SEQ ID NO: 17. In another specific embodiment, the amino acid sequence of the polypeptide is at least 99% identical to the amino acid sequence of SEQ ID NO: 17.

In a specific embodiment, the amino acid sequence of the polypeptide is at least 90% identical to the amino acid sequence of SEQ ID NO: 18. In another specific embodiment, the amino acid sequence of the polypeptide is at least 95% identical to the amino acid sequence of SEQ ID NO: 18. In another specific embodiment, the amino acid sequence of the polypeptide is at least 96% identical to the amino acid sequence of SEQ ID NO: 18. In another specific embodiment, the amino acid sequence of the polypeptide is at least 97% identical to the amino acid sequence of SEQ ID NO: 18. In another specific embodiment, the amino acid sequence of the polypeptide is at least 98% identical to the amino acid sequence of SEQ ID NO: 18. In another specific embodiment, the amino acid sequence of the polypeptide is at least 99% identical to the amino acid sequence of SEQ ID NO: 18.

In a specific embodiment, the amino acid sequence of the polypeptide is at least 90% identical to the amino acid sequence of SEQ ID NO: 34. In another specific embodiment, the amino acid sequence of the polypeptide is at least 95% identical to the amino acid sequence of SEQ ID NO: 34. In another specific embodiment, the amino acid sequence of the polypeptide is at least 96% identical to the amino acid sequence of SEQ ID NO: 34. In another specific embodiment, the amino acid sequence of the polypeptide is at least 97% identical to the amino acid sequence of SEQ ID NO: 34. In another specific embodiment, the amino acid sequence of the polypeptide is at least 98% identical to the amino acid sequence of SEQ ID NO: 34. In another specific embodiment, the amino acid sequence of the polypeptide is at least 99% identical to the amino acid sequence of SEQ ID NO: 34.

In a specific embodiment, the amino acid sequence of the polypeptide is at least 90% identical to the amino acid sequence of SEQ ID NO: 35. In another specific embodiment, the amino acid sequence of the polypeptide is at least 95% identical to the amino acid sequence of SEQ ID NO: 35. In another specific embodiment, the amino acid sequence of the polypeptide is at least 96% identical to the amino acid sequence of SEQ ID NO: 35. In another specific embodiment, the amino acid sequence of the polypeptide is at least 97% identical to the amino acid sequence of SEQ ID NO: 35. In another specific embodiment, the amino acid sequence of the polypeptide is at least 98% identical to the amino acid sequence of SEQ ID NO: 35. In another specific embodiment, the amino acid sequence of the polypeptide is at least 99% identical to the amino acid sequence of SEQ ID NO: 35.

In particular embodiments, the amino acid sequence of the polypeptide comprises or consists of the amino acid sequence of any one of SEQ ID NOs: 11 to 18, 34 and 35. In a specific embodiment, the amino acid sequence of the polypeptide comprises or consists of the amino acid sequence of SEQ ID NO: 11. In a specific embodiment, the amino acid sequence of the polypeptide comprises or consists of the amino acid sequence of SEQ ID NO: 12. In a specific embodiment, the amino acid sequence of the polypeptide comprises or consists of the amino acid sequence of SEQ ID NO: 13. In a specific embodiment, the amino acid sequence of the polypeptide comprises or consists of the amino acid sequence of SEQ ID NO: 14. In a specific embodiment, the amino acid sequence of the polypeptide comprises or consists of the amino acid sequence of SEQ ID NO: 15. In a specific embodiment, the amino acid sequence of the polypeptide comprises or consists of the amino acid sequence of SEQ ID NO: 16. In a specific embodiment, the amino acid sequence of the polypeptide comprises or consists of the amino acid sequence of SEQ ID NO: 17. In a specific embodiment, the amino acid sequence of the polypeptide comprises or consists of the amino acid sequence of SEQ ID NO: 18. In a specific embodiment, the amino acid sequence of the polypeptide comprises or consists of the amino acid sequence of SEQ ID NO: 34. In a specific embodiment, the amino acid sequence of the polypeptide comprises or consists of the amino acid sequence of SEQ ID NO: 35.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 2 and encodes the amino acid sequence of SEQ ID NO: 11. In a particular embodiment, the optimized nucleotide sequence is at least 90% identical to the nucleic acid sequence of SEQ ID NO: 2 and encodes the amino acid sequence of SEQ ID NO: 11. In another particular embodiment, the optimized nucleotide sequence is at least 95% identical to the nucleic acid sequence of SEQ ID NO: 2 and encodes the amino acid sequence of SEQ ID NO: 11. In another particular embodiment, the optimized nucleotide sequence is at least 96% identical to the nucleic acid sequence of SEQ ID NO: 2 and encodes the amino acid sequence of SEQ ID NO: 11. In another particular embodiment, the optimized nucleotide sequence is at least 97% identical to the nucleic acid sequence of SEQ ID NO: 2 and encodes the amino acid sequence of SEQ ID NO: 11. In another particular embodiment, the optimized nucleotide sequence is at least 98% identical to the nucleic acid sequence of SEQ ID NO: 2 and encodes the amino acid sequence of SEQ ID NO: 11. In another particular embodiment, the optimized nucleotide sequence is at least 99% identical to the nucleic acid sequence of SEQ ID NO: 2 and encodes the amino acid sequence of SEQ ID NO: 11. In a specific embodiment, the optimized nucleotide sequence comprises the nucleic acid sequence of SEQ ID NO: 2 and encodes the amino acid sequence of SEQ ID NO: 11. In another specific embodiment, the optimized nucleotide sequence consists of the nucleic acid sequence of SEQ ID NO: 2 and encodes the amino acid sequence of SEQ ID NO: 11.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 3 and encodes the amino acid sequence of SEQ ID NO: 12. In a particular embodiment, the optimized nucleotide sequence is at least 90% identical to the nucleic acid sequence of SEQ ID NO: 3 and encodes the amino acid sequence of SEQ ID NO: 12. In another particular embodiment, the optimized nucleotide sequence is at least 95% identical to the nucleic acid sequence of SEQ ID NO: 3 and encodes the amino acid sequence of SEQ ID NO: 12. In another particular embodiment, the optimized nucleotide sequence is at least 96% identical to the nucleic acid sequence of SEQ ID NO: 3 and encodes the amino acid sequence of SEQ ID NO: 12. In another particular embodiment, the optimized nucleotide sequence is at least 97% identical to the nucleic acid sequence of SEQ ID NO: 3 and encodes the amino acid sequence of SEQ ID NO: 12. In another particular embodiment, the optimized nucleotide sequence is at least 98% identical to the nucleic acid sequence of SEQ ID NO: 3 and encodes the amino acid sequence of SEQ ID NO: 12. In another particular embodiment, the optimized nucleotide sequence is at least 99% identical to the nucleic acid sequence of SEQ ID NO: 3 and encodes the amino acid sequence of SEQ ID NO: 12. In a specific embodiment, the optimized nucleotide sequence comprises the nucleic acid sequence of SEQ ID NO: 3 and encodes the amino acid sequence of SEQ ID NO: 12. In another specific embodiment, the optimized nucleotide sequence consists of the nucleic acid sequence of SEQ ID NO: 3 and encodes the amino acid sequence of SEQ ID NO: 12.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 4 and encodes the amino acid sequence of SEQ ID NO: 13. In a particular embodiment, the optimized nucleotide sequence is at least 90% identical to the nucleic acid sequence of SEQ ID NO: 4 and encodes the amino acid sequence of SEQ ID NO: 13. In another particular embodiment, the optimized nucleotide sequence is at least 95% identical to the nucleic acid sequence of SEQ ID NO: 4 and encodes the amino acid sequence of SEQ ID NO: 13. In another particular embodiment, the optimized nucleotide sequence is at least 96% identical to the nucleic acid sequence of SEQ ID NO: 4 and encodes the amino acid sequence of SEQ ID NO: 13. In another particular embodiment, the optimized nucleotide sequence is at least 97% identical to the nucleic acid sequence of SEQ ID NO: 4 and encodes the amino acid sequence of SEQ ID NO: 13. In another particular embodiment, the optimized nucleotide sequence is at least 98% identical to the nucleic acid sequence of SEQ ID NO: 4 and encodes the amino acid sequence of SEQ ID NO: 13. In another particular embodiment, the optimized nucleotide sequence is at least 99% identical to the nucleic acid sequence of SEQ ID NO: 4 and encodes the amino acid sequence of SEQ ID NO: 13. In a specific embodiment, the optimized nucleotide sequence comprises the nucleic acid sequence of SEQ ID NO: 4 and encodes the amino acid sequence of SEQ ID NO: 13. In another specific embodiment, the optimized nucleotide sequence consists of the nucleic acid sequence of SEQ ID NO: 4 and encodes the amino acid sequence of SEQ ID NO: 13.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 5 and encodes the amino acid sequence of SEQ ID NO: 14. In a particular embodiment, the optimized nucleotide sequence is at least 90% identical to the nucleic acid sequence of SEQ ID NO: 5 and encodes the amino acid sequence of SEQ ID NO: 14. In another particular embodiment, the optimized nucleotide sequence is at least 95% identical to the nucleic acid sequence of SEQ ID NO: 5 and encodes the amino acid sequence of SEQ ID NO: 14. In another particular embodiment, the optimized nucleotide sequence is at least 96% identical to the nucleic acid sequence of SEQ ID NO: 5 and encodes the amino acid sequence of SEQ ID NO: 14. In another particular embodiment, the optimized nucleotide sequence is at least 97% identical to the nucleic acid sequence of SEQ ID NO: 5 and encodes the amino acid sequence of SEQ ID NO: 14. In another particular embodiment, the optimized nucleotide sequence is at least 98% identical to the nucleic acid sequence of SEQ ID NO: 5 and encodes the amino acid sequence of SEQ ID NO: 14. In another particular embodiment, the optimized nucleotide sequence is at least 99% identical to the nucleic acid sequence of SEQ ID NO: 5 and encodes the amino acid sequence of SEQ ID NO: 14. In a specific embodiment, the optimized nucleotide sequence comprises the nucleic acid sequence of SEQ ID NO: 5 and encodes the amino acid sequence of SEQ ID NO: 14. In another specific embodiment, the optimized nucleotide sequence consists of the nucleic acid sequence of SEQ ID NO: 5 and encodes the amino acid sequence of SEQ ID NO: 14.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 6 and encodes the amino acid sequence of SEQ ID NO: 15. In a particular embodiment, the optimized nucleotide sequence is at least 90% identical to the nucleic acid sequence of SEQ ID NO: 6 and encodes the amino acid sequence of SEQ ID NO: 15. In another particular embodiment, the optimized nucleotide sequence is at least 95% identical to the nucleic acid sequence of SEQ ID NO: 6 and encodes the amino acid sequence of SEQ ID NO: 15. In another particular embodiment, the optimized nucleotide sequence is at least 96% identical to the nucleic acid sequence of SEQ ID NO: 6 and encodes the amino acid sequence of SEQ ID NO: 15. In another particular embodiment, the optimized nucleotide sequence is at least 97% identical to the nucleic acid sequence of SEQ ID NO: 6 and encodes the amino acid sequence of SEQ ID NO: 15. In another particular embodiment, the optimized nucleotide sequence is at least 98% identical to the nucleic acid sequence of SEQ ID NO: 6 and encodes the amino acid sequence of SEQ ID NO: 15. In another particular embodiment, the optimized nucleotide sequence is at least 99% identical to the nucleic acid sequence of SEQ ID NO: 6 and encodes the amino acid sequence of SEQ ID NO: 15. In a specific embodiment, the optimized nucleotide sequence comprises the nucleic acid sequence of SEQ ID NO: 6 and encodes the amino acid sequence of SEQ ID NO: 15. In another specific embodiment, the optimized nucleotide sequence consists of the nucleic acid sequence of SEQ ID NO: 6 and encodes the amino acid sequence of SEQ ID NO: 15.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 7 and encodes the amino acid sequence of SEQ ID NO: 16. In a particular embodiment, the optimized nucleotide sequence is at least 90% identical to the nucleic acid sequence of SEQ ID NO: 7 and encodes the amino acid sequence of SEQ ID NO: 16. In another particular embodiment, the optimized nucleotide sequence is at least 95% identical to the nucleic acid sequence of SEQ ID NO: 7 and encodes the amino acid sequence of SEQ ID NO: 16. In another particular embodiment, the optimized nucleotide sequence is at least 96% identical to the nucleic acid sequence of SEQ ID NO: 7 and encodes the amino acid sequence of SEQ ID NO: 16. In another particular embodiment, the optimized nucleotide sequence is at least 97% identical to the nucleic acid sequence of SEQ ID NO: 7 and encodes the amino acid sequence of SEQ ID NO: 16. In another particular embodiment, the optimized nucleotide sequence is at least 98% identical to the nucleic acid sequence of SEQ ID NO: 7 and encodes the amino acid sequence of SEQ ID NO: 16. In another particular embodiment, the optimized nucleotide sequence is at least 99% identical to the nucleic acid sequence of SEQ ID NO: 7 and encodes the amino acid sequence of SEQ ID NO: 16. In a specific embodiment, the optimized nucleotide sequence comprises the nucleic acid sequence of SEQ ID NO: 7 and encodes the amino acid sequence of SEQ ID NO: 16. In another specific embodiment, the optimized nucleotide sequence consists of the nucleic acid sequence of SEQ ID NO: 7 and encodes the amino acid sequence of SEQ ID NO: 16.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 8 and encodes the amino acid sequence of SEQ ID NO: 17. In a particular embodiment, the optimized nucleotide sequence is at least 90% identical to the nucleic acid sequence of SEQ ID NO: 8 and encodes the amino acid sequence of SEQ ID NO: 17. In another particular embodiment, the optimized nucleotide sequence is at least 95% identical to the nucleic acid sequence of SEQ ID NO: 8 and encodes the amino acid sequence of SEQ ID NO: 17. In another particular embodiment, the optimized nucleotide sequence is at least 96% identical to the nucleic acid sequence of SEQ ID NO: 8 and encodes the amino acid sequence of SEQ ID NO: 17. In another particular embodiment, the optimized nucleotide sequence is at least 97% identical to the nucleic acid sequence of SEQ ID NO: 8 and encodes the amino acid sequence of SEQ ID NO: 17. In another particular embodiment, the optimized nucleotide sequence is at least 98% identical to the nucleic acid sequence of SEQ ID NO: 8 and encodes the amino acid sequence of SEQ ID NO: 17. In another particular embodiment, the optimized nucleotide sequence is at least 99% identical to the nucleic acid sequence of SEQ ID NO: 8 and encodes the amino acid sequence of SEQ ID NO: 17. In a specific embodiment, the optimized nucleotide sequence comprises the nucleic acid sequence of SEQ ID NO: 8 and encodes the amino acid sequence of SEQ ID NO: 17. In another specific embodiment, the optimized nucleotide sequence consists of the nucleic acid sequence of SEQ ID NO: 8 and encodes the amino acid sequence of SEQ ID NO: 17.

In some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 9 and encodes the amino acid sequence of SEQ ID NO: 18. In a particular embodiment, the optimized nucleotide sequence is at least 90% identical to the nucleic acid sequence of SEQ ID NO: 9 and encodes the amino acid sequence of SEQ ID NO: 18. In another particular embodiment, the optimized nucleotide sequence is at least 95% identical to the nucleic acid sequence of SEQ ID NO: 9 and encodes the amino acid sequence of SEQ ID NO: 18. In another particular embodiment, the optimized nucleotide sequence is at least 96% identical to the nucleic acid sequence of SEQ ID NO: 9 and encodes the amino acid sequence of SEQ ID NO: 18. In another particular embodiment, the optimized nucleotide sequence is at least 97% identical to the nucleic acid sequence of SEQ ID NO: 9 and encodes the amino acid sequence of SEQ ID NO: 18. In another particular embodiment, the optimized nucleotide sequence is at least 98% identical to the nucleic acid sequence of SEQ ID NO: 9 and encodes the amino acid sequence of SEQ ID NO: 18. In another particular embodiment, the optimized nucleotide sequence is at least 99% identical to the nucleic acid sequence of SEQ ID NO: 9 and encodes the amino acid sequence of SEQ ID NO: 18. In a specific embodiment, the optimized nucleotide sequence comprises the nucleic acid sequence of SEQ ID NO: 9 and encodes the amino acid sequence of SEQ ID NO: 18. In another specific embodiment, the optimized nucleotide sequence consists of the nucleic acid sequence of SEQ ID NO: 9 and encodes the amino acid sequence of SEQ ID NO: 18.

The data presented in the examples of the present application support the notion that fusing an engineered variant human ACE2 polypeptide (in particular that encoded by SEQ ID NO: 4 or 5) to an Fc region (e.g., the one encoded by SEQ ID NO: 27) to yield an ACE2-fusion protein would result in a more effective neutralization of viruses that must bind to ACE2 protein in order to gain entry into cells (e.g., various SARS-CoV2 variants, as exemplified herein). Accordingly, in some embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 32 and encodes the amino acid sequence of SEQ ID NO: 34. In a particular embodiment, the optimized nucleotide sequence is at least 90% identical to the nucleic acid sequence of SEQ ID NO: 32 and encodes the amino acid sequence of SEQ ID NO: 34. In another particular embodiment, the optimized nucleotide sequence is at least 95% identical to the nucleic acid sequence of SEQ ID NO: 32 and encodes the amino acid sequence of SEQ ID NO: 34. In another particular embodiment, the optimized nucleotide sequence is at least 96% identical to the nucleic acid sequence of SEQ ID NO: 32 and encodes the amino acid sequence of SEQ ID NO: 34. In another particular embodiment, the optimized nucleotide sequence is at least 97% identical to the nucleic acid sequence of SEQ ID NO: 32 and encodes the amino acid sequence of SEQ ID NO: 34. In another particular embodiment, the optimized nucleotide sequence is at least 98% identical to the nucleic acid sequence of SEQ ID NO: 32 and encodes the amino acid sequence of SEQ ID NO: 34. In another particular embodiment, the optimized nucleotide sequence is at least 99% identical to the nucleic acid sequence of SEQ ID NO: 32 and encodes the amino acid sequence of SEQ ID NO: 34. In a specific embodiment, the optimized nucleotide sequence comprises the nucleic acid sequence of SEQ ID NO: 32 and encodes the amino acid sequence of SEQ ID NO: 34. In another specific embodiment, the optimized nucleotide sequence consists of the nucleic acid sequence of SEQ ID NO: 32 and encodes the amino acid sequence of SEQ ID NO: 34. In other embodiments, the optimized nucleotide sequence is at least 60%, 65%, 70%, 75%, 80%, 85%, 90%, 95%, 96%, 97%, 98%, or 99% identical to, comprises, or consists of the nucleic acid sequence of SEQ ID NO: 33 and encodes the amino acid sequence of SEQ ID NO: 35. In a particular embodiment, the optimized nucleotide sequence is at least 90% identical to the nucleic acid sequence of SEQ ID NO: 33 and encodes the amino acid sequence of SEQ ID NO: 35. In another particular embodiment, the optimized nucleotide sequence is at least 95% identical to the nucleic acid sequence of SEQ ID NO: 33 and encodes the amino acid sequence of SEQ ID NO: 35. In another particular embodiment, the optimized nucleotide sequence is at least 96% identical to the nucleic acid sequence of SEQ ID NO: 33 and encodes the amino acid sequence of SEQ ID NO: 35. In another particular embodiment, the optimized nucleotide sequence is at least 97% identical to the nucleic acid sequence of SEQ ID NO: 33 and encodes the amino acid sequence of SEQ ID NO: 35. In another particular embodiment, the optimized nucleotide sequence is at least 98% identical to the nucleic acid sequence of SEQ ID NO: 33 and encodes the amino acid sequence of SEQ ID NO: 35. In another particular embodiment, the optimized nucleotide sequence is at least 99% identical to the nucleic acid sequence of SEQ ID NO: 33 and encodes the amino acid sequence of SEQ ID NO: 35. In a specific embodiment, the optimized nucleotide sequence comprises the nucleic acid sequence of SEQ ID NO: 33 and encodes the amino acid sequence of SEQ ID NO: 35. In another specific embodiment, the optimized nucleotide sequence consists of the nucleic acid sequence of SEQ ID NO: 33 and encodes the amino acid sequence of SEQ ID NO: 35.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. In a specific embodiment, an alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489.

### Polypeptides Comprising an Immunoglobulin Fc Region

In some embodiments, a polypeptide encoded by an mRNA of the invention comprises the extracellular domain of human ACE2 protein linked to an immunoglobulin fragment crystallizable (Fc) region. Such a polypeptide may be particularly useful when intravenous route is used to administer the mRNA of the invention (e.g., encapsulated in a lipid nanoparticle). The immunoglobulin Fc region, when linked to a polypeptide, increases its half-life in systemic circulation. This makes it possible to administer an mRNA of the invention systemically, typically by intravenous administration. Cells accessible through systemic delivery (primarily the liver, and to some extent the spleen) are transfected by the mRNA, which is then expressed. The expressed polypeptides are secreted into the circulation (i.e., they may be detected in the serum) and may be taken up by target cells in tissues remote to the site of mRNA expression, e.g., the lungs. Polypeptides encoded by an mRNA of the invention comprising the extracellular domain of human ACE2 protein linked to an immunoglobulin Fc region may be particularly effective in neutralizing viruses which use the human ACE2 protein for cellular entry, e.g., due to higher avidity.

In particular embodiments, the polypeptide encoded by an mRNA of the invention comprises an immunoglobulin Fc region at the C-terminus of the extracellular domain of human ACE2 protein. In some embodiments, the extracellular domain of human ACE2 protein and the immunoglobulin Fc region are linked by a linker sequence, e.g., GGGGS (SEQ ID NO: 28). The immunoglobulin Fc region can be derived from IgG1, IgG2, or IgG4. In a specific embodiment, the immunoglobulin Fc region is derived from human IgG1. For example, the immunoglobulin Fc region may comprise the amino acid sequence of SEQ ID NO: 27 (which is derived from human IgG1):

PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK.

Polypeptides Comprising Localization Sequences and/or Linker Sequences

In some embodiments, the polypeptide encoded by an mRNA of the invention comprises a localization sequence which facilitates the delivery of the polypeptide to a target cell in a tissue of therapeutic interest (e.g., the lungs). In some embodiments, the localization sequence is an amino acid sequence which facilitates transcytosis of the extracellular domain of human ACE2 protein across an epithelium. For example, the localization sequence may facilitate transport of the extracellular domain of human ACE2 protein across a layer of airway epithelial cells for effective delivery to the airways or the lumen of the lungs. Typically, the localization sequence is exogenous to the naturally occurring human ACE2 protein encoded by SEQ ID NO: 10. In some embodiments, the extracellular domain of human ACE2 protein may include an endogenous localization sequence.

A localization sequence (in particular an exogenous localization sequence) may be at the carboxy-terminus (C-terminus) of the extracellular domain of human ACE2 protein within a polypeptide encoded by the mRNA of the invention. The localization sequence may be linked to the extracellular domain of human ACE2 protein by a linker sequence, e.g., GGGGS (SEQ ID NO: 28).

In particular embodiments, the polypeptide encoded by an mRNA of the invention comprises the extracellular domain of human ACE2 protein, an immunoglobulin Fc region and a localization sequence (typically in this order from N-terminus to C-terminus). In particular embodiments, the extracellular domain of human ACE2 protein, the immunoglobulin Fc region and the localization sequence within the polypeptide are linked by one or more linker sequences, e.g., GGGGS (SEQ ID NO: 28).

In some embodiments, the localization sequence is a KLKL localization sequence. In specific embodiments, the KLKL localization sequence comprises the amino acid sequence of SEQ ID NO: 29: QRNPKLKLIRRHPTLRIPPI.

In some embodiments, the localization sequence is a RLRL localization sequence. In specific embodiments, the RLRL localization sequence comprises the amino acid sequence of SEQ ID NO: 30: QRNPRLRLIRRHPTLRIPPI.

In some embodiments, the localization sequence is an immunoglobulin J chain. In specific embodiments, the immunoglobulin J chain comprises the amino acid sequence of SEQ ID NO: 31:

QEDERIVLVDNKCKSARITSRIIRSSEDPNEDIVERNIRIIVPLNNREN
ISDPTSPLRTRFVYHLSDLSKKCDPTEVELDNQIVTATQSNICDEDSAT
ETCYTYDRNKCYTAVVPLVYGGETKMVETALTPDACYPD.

The KLKL localization sequence, RLRL localization sequence, and J chain bind to the polymeric Ig receptor (pIgR) which are expressed on the surface of lung epithelial cells. The localization sequences can facilitate transcytosis across the airway epithelium, such that polypeptides including one of these sequences may be more effectively delivered to the lung lumen, where they are available to bind to and neutralize viral surface proteins of infectious virus particles that use the human ACE2 protein for cellular entry (Borrok et al., JCI Insight 2018 Jun. 21; 3(12): e97844).

In particular embodiments, a polypeptide encoded by an mRNA of the invention which comprises the extracellular domain of human ACE2 protein, a localization sequence and optionally a immunoglobulin Fc region, also comprises one or more amino acid substitutions that increase the binding affinity for SARS-CoV-2 spike (S) glycoprotein relative to a naturally occurring human ACE2 protein. For example, the extracellular domain of human ACE2 protein encoded by SEQ ID NO: 13 may be combined with a localization sequence (e.g., a KLKL localization sequence or a RLRL localization sequence) and optionally an immunoglobulin Fc region (e.g., an immunoglobulin Fc region derived from human IgG1 such as the one encoded by the amino acid sequence of SEQ ID NO: 27) to form a single fusion protein. Similarly, the extracellular domain of human ACE2 protein encoded by SEQ ID NO: 14 may be combined with a localization sequence (e.g., a KLKL localization sequence or a RLRL localization sequence) and optionally an immunoglobulin Fc region (e.g., an immunoglobulin Fc region derived from human IgG1 such as the one encoded by the amino acid sequence of SEQ ID NO: 27) to form a single fusion protein. Alternatively, the extracellular domain of human ACE2 protein encoded by SEQ ID NO: 15 may be combined with a localization sequence (e.g., a KLKL localization sequence or a RLRL localization sequence) and optionally an immunoglobulin Fc region (e.g., an immunoglobulin Fc region derived from human IgG1 such as the one encoded by the amino acid sequence of SEQ ID NO: 27) to form a single fusion protein. In the fusion protein, the extracellular domain of human ACE2 protein, the localization sequence and optionally the immunoglobulin Fc region may be linked by a linker sequence (e.g., SEQ ID NO: 28).

The amino acid sequences of an exemplary immunoglobulin Fc region, exemplary linker sequence, and exemplary localization sequences are shown below in Table 1.

TABLE 1
Amino acid sequences of exemplary immunoglobulin Fc region, exemplary
and exemplary localization sequences

| | SEQ ID | |
|---|---|---|
| NAME | NO: | SEQUENCE |
| Immunoglobulin | 27 | PKSCDKTHTCPPCPAPELLGGPSVFLFPPKPKDTLMISR |
| Fc region from | | TPEVTCVVVDVSHEDPEVKFNWYVDGVEVHNAKTKP |
| human IgG1 | | REEQYNSTYRVVSVLTVLHQDWLNGKEYKCKVSNK |
| | | ALPAPIEKTISKAKGQPREPQVYTLPPSRDELTKNQVS |
| | | LTCLVKGFYPSDIAVEWESNGQPENNYKTTPPVLDSD |
| | | GSFFLYSKLTVDKSRWQQGNVFSCSVMHEALHNHYT |
| | | QKSLSLSPGK |
| GS linker | 28 | GGGGS |
| KLKL | 29 | QRNPKLKLIRRHPTLRIPPI |
| localization | | |
| sequence | | |
| RLRL | 30 | QRNPRLRLIRRHPTLRIPPI |
| localization | | |
| sequence | | |
| Immunoglobulin J | 31 | QEDERIVLVDNKCKSARITSRIIRSSEDPNEDIVERNIRII |
| chain | | VPLNNRENISDPTSPLRTRFVYHLSDLSKKCDPTEVEL |
| | | DNQIVTATQSNICDEDSATETCYTYDRNKCYTAVVPL |
| | | VYGGETKMVETALTPDACYPD |

### Multimeric Constructs

In certain embodiments, the polypeptide comprises two extracellular domains of human ACE2 protein or portions thereof operably linked to form a dimer. Such polypeptides comprising two extracellular domains of human ACE2 protein can function as a ‘bivalent decoy’, wherein each monomeric extracellular domain of human ACE2 protein can bind to a viral surface protein, such as the spike (5) glycoprotein of SARS-CoV-2 (Linsky et al., Science. 2020 Dec. 4; 370(6521): pp. 1208-1214).

### Exemplary Polypeptides Comprising the Extracellular Domain of Human ACE2 Protein

The amino acid sequences of exemplary polypeptides comprising the extracellular domain of human ACE2 protein, encoded by mRNAs comprising optimized nucleotide sequences of the invention, are shown in Table 2.

TABLE 2
Amino acid sequences of exemplary polypeptides comprising the extracellular
domain of human ACE2 encoded by optimized nucleotide sequences

| | SEQ ID | |
|---|---|---|
| NAME | NO: | SEQUENCE |
| Reference | 10 | MSSSSWLLLSLVAVTAAQSTIEEQAKTFLDKFNHEAE |
| human ACE2 | | DLFYQSSLASWNYNTNITEENVQNMNNAGDKWSAFL |
| protein | | KEQSTLAQMYPLQEIQNLTVKLQLQALQQNGSSVLSE |
| sequence | | DKSKRLNTILNTMSTIYSTGKVCNPDNPQECLLLEPGL |
| (obtained from | | NEIMANSLDYNERLWAWESWRSEVGKQLRPLYEEYV |
| NCBI Genbank | | VLKNEMARANHYEDYGDYWRGDYEVNGVDGYDYS |
| Reference | | RGQLIEDVEHTFEEIKPLYEHLHAYVRAKLMNAYPSYI |
| Sequence | | SPIGCLPAHLLGDMWGRFWTNLYSLTVPFGQKPNIDV |
| NM_001371415.1) | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVSIWLIVFGVVMGVIVVGIVILIFTGIRDRKK |
| | | KNKARSGENPYASIDISKGENNPGFQNTDDVQTSF |
| hsACE2 | 11 | MSSSSWLLLSLVAVTAAQSTIEEQAKTFLDKFNHEAE |
| | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVS |
| hsACE2-GS- | 12 | MSSSSWLLLSLVAVTAAQSTIEEQAKTFLDKFNHEAE |
| Fc | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVSGGGGSPKSCDKTHTCPPCPAPELLGGPSV |
| | | FLFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQVY |
| | | TLPPSRDELTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGK |
| hsACE2 N90D | 13 | MSSSSWLLLSLVAVTAAQSTIEEQAKTFLDKFNHEAE |
| R273A | | DLFYQSSLASWNYNTNITEENVQNMNNAGDKWSAFL |
| | | KEQSTLAQMYPLQEIQDLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGAFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVS |
| hsACE2 N90D | 14 | MSSSSWLLLSLVAVTAAQSTIEEQAKTFLDKFNHEAE |
| T92A | | DLFYQSSLASWNYNTNITEENVQNMNNAGDKWSAFL |
| | | KEQSTLAQMYPLQEIQDLAVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVS |
| hsACE2 | 15 | MSSSSWLLLSLVAVTAAQSTIEEQAKTFLDKFNHEAE |
| H374N H378N | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHNEMGNIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVS |
| hsACE2-GS- | 16 | MSSSSWLLLSLVAVTAAQSTIEEQAKTFLDKFNHEAE |
| Fc-GS-KLKL | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVSGGGGSPKSCDKTHTCPPCPAPELLGGPSV |
| | | FLFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQVY |
| | | TLPPSRDELTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGKGGGGSQRNPKLKLI |
| | | RRHPTLRIPPI |
| hsACE2-GS- | 17 | MSSSSWLLLSLVAVTAAQSTIEEQAKTFLDKFNHEAE |
| Fc-GS-RLRL | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVSGGGGSPKSCDKTHTCPPCPAPELLGGPSV |
| | | FLFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQVY |
| | | TLPPSRDELTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGKGGGGSQRNPRLRLI |
| | | RRHPTLRIPPI |
| hsACE2-GS- | 18 | MSSSSWLLLSLVAVTAAQSTIEEQAKTFLDKFNHEAE |
| Fc-GS-J chain | | DLFYQSSLASWNYNTNITEENVQNMNNAGDKWSAFL |
| | | KEQSTLAQMYPLQEIQNLTVKLQLQALQQNGSSVLSE |
| | | DKSKRLNTILNTMSTIYSTGKVCNPDNPQECLLLEPGL |
| | | NEIMANSLDYNERLWAWESWRSEVGKQLRPLYEEYV |
| | | VLKNEMARANHYEDYGDYWRGDYEVNGVDGYDYS |
| | | RGQLIEDVEHTFEEIKPLYEHLHAYVRAKLMNAYPSYI |
| | | SPIGCLPAHLLGDMWGRFWTNLYSLTVPFGQKPNIDV |
| | | TDAMVDQAWDAQRIFKEAEKFFVSVGLPNMTQGFW |
| | | ENSMLTDPGNVQKAVCHPTAWDLGKGDFRILMCTKV |
| | | TMDDFLTAHHEMGHIQYDMAYAAQPFLLRNGANEG |
| | | FHEAVGEIMSLSAATPKHLKSIGLLSPDFQEDNETEINF |
| | | LLKQALTIVGTLPFTYMLEKWRWMVFKGEIPKDQWM |
| | | KKWWEMKREIVGVVEPVPHDETYCDPASLFHVSNDY |
| | | SFIRYYTRTLYQFQFQEALCQAAKHEGPLHKCDISNST |
| | | EAGQKLFNMLRLGKSEPWTLALENVVGAKNMNVRP |
| | | LLNYFEPLFTWLKDQNKNSFVGWSTDWSPYADQSIK |
| | | VRISLKSALGDKAYEWNDNEMYLFRSSVAYAMRQYF |
| | | LKVKNQMILFGEEDVRVANLKPRISFNFFVTAPKNVS |
| | | DIIPRTEVEKAIRMSRSRINDAFRLNDNSLEFLGIQPTL |
| | | GPPNQPPVSGGGGSPKSCDKTHTCPPCPAPELLGGPSV |
| | | FLFPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALPAPIEKTISKAKGQPREPQVY |
| | | TLPPSRDELTKNQVSLTCLVKGFYPSDIAVEWESNGQP |
| | | ENNYKTTPPVLDSDGSFFLYSKLTVDKSRWQQGNVFS |
| | | CSVMHEALHNHYTQKSLSLSPGKGGGGSQEDERIVLV |
| | | DNKCKSARITSRIIRSSEDPNEDIVERNIRIIVPLNNRENI |
| | | SDPTSPLRTRFVYHLSDLSKKCDPTEVELDNQIVTATQ |
| | | SNICDEDSATETCYTYDRNKCYTAVVPLVYGGETKM |
| | | VETALTPDACYPD |
| hsACE2-GS- | 34 | MSSSSWLLLSLVAVTAAQSTIEEQAKTFLDKFNHEAE |
| Fc wherein the | | DLFYQSSLASWNYNTNITEENVQNMNNAGDKWS |
| hsACE2 is | | AFLKEQSTLAQMYPLQEIQDLTVKLQLQALQQNGSSV |
| hsACE2 N90D | | LSEDKSKRLNTILNTMSTIYSTGKVCNPDNPQE |
| R273A | | CLLLEPGLNEIMANSLDYNERLWAWESWRSEVGKQL |
| | | RPLYEEYVVLKNEMARANHYEDYGDYWRGDYEVN |
| | | GVDGYDYSRGQLIEDVEHTFEEIKPLYEHLHAYVRAK |
| | | LMNAYPSYISPIGCLPAHLLGDMWGAFWTNLYS |
| | | LTVPFGQKPNIDVTDAMVDQAWDAQRIFKEAEKFFVS |
| | | VGLPNMTQGFWENSMLTDPGNVQKAVCHPTAWD |
| | | LGKGDFRILMCTKVTMDDFLTAHHEMGHIQYDMAY |
| | | AAQPFLLRNGANEGFHEAVGEIMSLSAATPKHLKS |
| | | IGLLSPDFQEDNETEINFLLKQALTIVGTLPFTYMLEK |
| | | WRWMVFKGEIPKDQWMKKWWEMKREIVGVVEP |
| | | VPHDETYCDPASLFHVSNDYSFIRYYTRTLYQFQFQE |
| | | ALCQAAKHEGPLHKCDISNSTEAGQKLFNMLRL |
| | | GKSEPWTLALENVVGAKNMNVRPLLNYFEPLFTWLK |
| | | DQNKNSFVGWSTDWSPYADQSIKVRISLKSALGD |
| | | KAYEWNDNEMYLFRSSVAYAMRQYFLKVKNQMILF |
| | | GEEDVRVANLKPRISFNFFVTAPKNVSDIIPRTEV |
| | | EKAIRMSRSRINDAFRLNDNSLEFLGIQPTLGPPNQPPV |
| | | SGGGGSPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSRDE |
| | | LTKNQVSLTCLVKGFYPSDIAVEWESNGQPENNYKTT |
| | | PPVLDSDGSFFLYSKLTVDKSRWQQGNVFSCSVMHE |
| | | ALHNHYTQKSLSLSPGK |
| hsACE2-GS- | 35 | MSSSSWLLLSLVAVTAAQSTIEEQAKTFLDKFNHEAE |
| Fc wherein the | | DLFYQSSLASWNYNTNITEENVQNMNNAGDKWS |
| hsACE2 is | | AFLKEQSTLAQMYPLQEIQDLAVKLQLQALQQNGSSV |
| hsACE2 N90D | | LSEDKSKRLNTILNTMSTIYSTGKVCNPDNPQE |
| T92A | | CLLLEPGLNEIMANSLDYNERLWAWESWRSEVGKQL |
| | | RPLYEEYVVLKNEMARANHYEDYGDYWRGDYEVN |
| | | GVDGYDYSRGQLIEDVEHTFEEIKPLYEHLHAYVRAK |
| | | LMNAYPSYISPIGCLPAHLLGDMWGRFWTNLYS |
| | | LTVPFGQKPNIDVTDAMVDQAWDAQRIFKEAEKFFVS |
| | | VGLPNMTQGFWENSMLTDPGNVQKAVCHPTAWD |
| | | LGKGDFRILMCTKVTMDDFLTAHHEMGHIQYDMAY |
| | | AAQPFLLRNGANEGFHEAVGEIMSLSAATPKHLKS |
| | | IGLLSPDFQEDNETEINFLLKQALTIVGTLPFTYMLEK |
| | | WRWMVFKGEIPKDQWMKKWWEMKREIVGVVEP |
| | | VPHDETYCDPASLFHVSNDYSFIRYYTRTLYQFQFQE |
| | | ALCQAAKHEGPLHKCDISNSTEAGQKLFNMLRL |
| | | GKSEPWTLALENVVGAKNMNVRPLLNYFEPLFTWLK |
| | | DQNKNSFVGWSTDWSPYADQSIKVRISLKSALGD |
| | | KAYEWNDNEMYLFRSSVAYAMRQYFLKVKNQMILF |
| | | GEEDVRVANLKPRISFNFFVTAPKNVSDIIPRTEV |
| | | EKAIRMSRSRINDAFRLNDNSLEFLGIQPTLGPPNQPPV |
| | | SGGGGSPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEY |
| | | KCKVSNKALPAPIEKTISKAKGQPREPQVYTLPPSRDE |
| | | LTKNQVSLTCLVKGFYPSDIAVEWESNGQPENNYKTT |
| | | PPVLDSDGSFFLYSKLTVDKSRWQQGNVFSCSVMHE |
| | | ALHNHYTQKSLSLSPGK |

### Generation of Optimized Nucleotide Sequences

The present invention provides mRNAs that comprise optimized nucleotide sequence encoding a polypeptide comprising the extracellular domain of human angiotensin-converting enzyme 2 (ACE2) protein, or a portion thereof, which binds to a viral surface protein. These mRNAs are modified relative to their naturally occurring counterparts to (a) improve the yield of full-length mRNAs during in vitro synthesis, and (b) to maximize expression of the encoded polypeptide after delivery of the mRNA to a target cell in vivo. Sequence motifs that favour rapid degradation of the mRNA in the target cell have also been removed.

A high level of in vivo expression of polypeptides comprising the extracellular domain of human ACE2 protein is desirable because the more ‘decoy receptors’ are available to bind to a viral surface protein, the more viral particles can be neutralized, thereby preventing them from entering cells for viral replication.

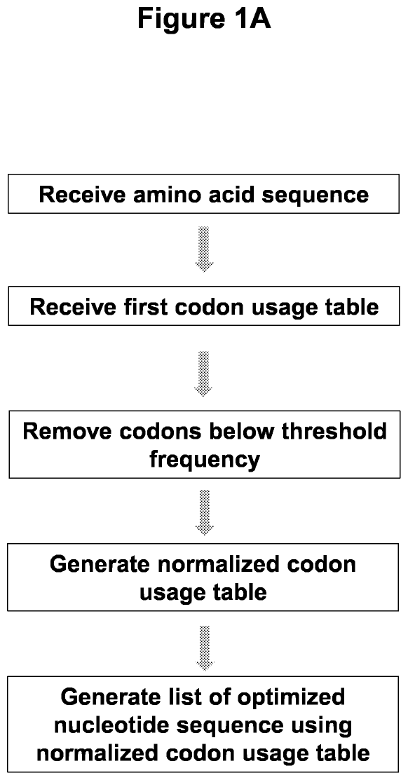

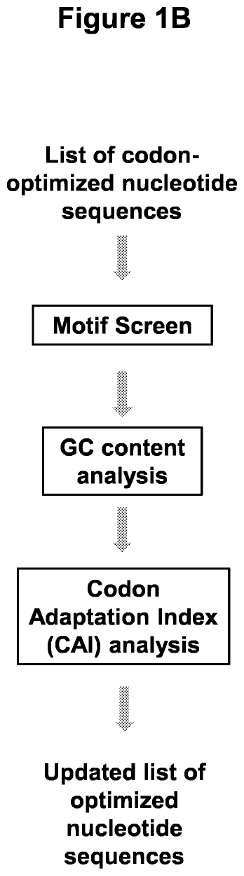

FIGS. 1A and 1B illustrate a process for generating optimized nucleotide sequences in accordance with the invention. The process first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded polypeptide.

### Codon Optimization

The genetic code has 64 possible codons. Each codon comprises a sequence of three nucleotides. The usage frequency for each codon in the protein-coding regions of the genome can be calculated by determining the number of instances that a specific codon appears within the protein-coding regions of the genome, and subsequently dividing the obtained value by the total number of codons that encode the same amino acid within protein-coding regions of the genome.

A codon usage table contains experimentally derived data regarding how often, for the particular biological source from which the table has been generated, each codon is used to encode a certain amino acid. This information is expressed, for each codon, as a percentage (0 to 100%), or fraction (0 to 1), of how often that codon is used to encode a certain amino acid relative to the total number of times a codon encodes that amino acid.

Codon usage tables are stored in publically available databases, such as the Codon Usage Database (Nakamura et al. (2000) Nucleic Acids Research 28(1), 292; available online at https://www.kazusa.or.jp/codon/), and the High-performance Integrated Virtual Environment-Codon Usage Tables (HIVE-CUTs) database (Athey et al., (2017), BMC Bioinformatics 18(1), 391; available online at http://hive.biochemistry.gwu.edu/review/codon).

During the first step of codon optimization, codons are removed from a first codon usage table which reflects the frequency of each codon in a given organism (e.g., a mammal or human) if they are associated with a codon usage frequency which is less than a threshold frequency (e.g., 10%). The codon usage frequencies of the codons not removed in the first step are normalized to generate a normalized codon usage table. An optimized nucleotide sequence encoding an amino acid sequence of interest is generated by selecting a codon for each amino acid in the amino acid sequence based on the usage frequency of the one or more codons associated with a given amino acid in the normalized codon usage table. The probability of selecting a certain codon for a given amino acid is equal to the usage frequency associated with the codon associated with this amino acid in the normalized codon usage table.

The codon-optimized sequences of the invention are generated by a computer-implemented method for generating an optimized nucleotide sequence. The method comprises: (i) receiving an amino acid sequence, wherein the amino acid sequence encodes a peptide, polypeptide, or protein; (ii) receiving a first codon usage table, wherein the first codon usage table comprises a list of amino acids, wherein each amino acid in the table is associated with at least one codon and each codon is associated with a usage frequency; (iii) removing from the codon usage table any codons associated with a usage frequency which is less than a threshold frequency; (iv) generating a normalized codon usage table by normalizing the usage frequencies of the codons not removed in step (iii); and (v) generating an optimized nucleotide sequence encoding the amino acid sequence by selecting a codon for each amino acid in the amino acid sequence based on the usage frequency of the one or more codons associated with the amino acid in the normalized codon usage table. The threshold frequency can be in the range of 5%-30%, in particular 5%, 10%, 15%, 20%, 25%, or 30%. In the context of the present invention, the threshold frequency is typically 10%.

The step of generating a normalized codon usage table comprises: (a) distributing the usage frequency of each codon associated with a first amino acid and removed in step (iii) to the remaining codons associated with the first amino acid; and (b) repeating step (a) for each amino acid to produce a normalized codon usage table. In some embodiments, the usage frequency of the removed codons is distributed equally amongst the remaining codons. In some embodiments, the usage frequency of the removed codons is distributed amongst the remaining codons proportionally based on the usage frequency of each remaining codon. “Distributed” in this context may be defined as taking the combined magnitude of the usage frequencies of removed codons associated with a certain amino acid and apportioning some of this combined frequency to each of the remaining codons encoding the certain amino acid.

The step of selecting a codon for each amino acid comprises: (a) identifying, in the normalized codon usage table, the one or more codons associated with a first amino acid of the amino acid sequence; (b) selecting a codon associated with the first amino acid, wherein the probability of selecting a certain codon is equal to the usage frequency associated with the codon associated with the first amino acid in the normalized codon usage table; and (c) repeating steps (a) and (b) until a codon has been selected for each amino acid in the amino acid sequence.

The step of generating an optimized nucleotide sequence by selecting a codon for each amino acid in the amino acid sequence (step (v) in the above method) is performed n times to generate a list of optimized nucleotide sequences.

### Motif Screen

A motif screen filter is applied to the list of optimized nucleotide sequences. Optimized nucleotide sequences encoding any known negative cis-regulatory elements and negative repeat elements are removed from the list to generate an updated list.

For each optimized nucleotide sequence in the list, it is also determined whether it contains a termination signal. Any nucleotide sequence that contains one or more termination signals is removed from the list generating an updated list. In some embodiments, the termination signal has the following nucleotide sequence: 5′-X₁ATCTX₂TX₃-3′, wherein X₁, X₂ and X₃ are independently selected from A, C, T or G. In some embodiments, the termination signal has one of the following nucleotide sequences: TATCTGTT; and/or TTTTTT; and/or AAGCTT; and/or GAAGAGC; and/or TCTAGA. In a typical embodiment, the termination signal has the following nucleotide sequence: 5′-X₁AUCUX₂UX₃-3′, wherein X₁, X₂ and X₃ are independently selected from A, C, U or G. In a specific embodiment, the termination signal has one of the following nucleotide sequences: UAUCUGUU; and/or UUUUUU; and/or AAGCUU; and/or GAAGAGC; and/or UCUAGA.

### Guanine-Cytosine (GC) Content

The method further comprises determining a guanine-cytosine (GC) content of each of the optimized nucleotide sequences in the updated list of optimized nucleotide sequences. The GC content of a sequence is the percentage of bases in the nucleotide sequence that are guanine or cytosine. The list of optimized nucleotide sequences is further updated by removing any nucleotide sequence from the list, if its GC content falls outside a predetermined GC content range.

Determining a GC content of each of the optimized nucleotide sequences comprises, for each nucleotide sequence: determining a GC content of one or more additional portions of the nucleotide sequence, wherein the additional portions are non-overlapping with each other and with the first portion, and wherein updating the list of optimized sequences comprises: removing the nucleotide sequence if the GC content of any portion falls outside the predetermined GC content range, optionally wherein determining the GC content of the nucleotide sequence is halted when the GC content of any portion is determined to be outside the predetermined GC content range. In some embodiments, the first portion and/or the one or more additional portions of the nucleotide sequence comprise a predetermined number of nucleotides, optionally wherein the predetermined number of nucleotides is in the range of: 5 to 300 nucleotides, or 10 to 200 nucleotides, or 15 to 100 nucleotides, or 20 to 50 nucleotides. In the context of the present invention, the predetermined number of nucleotides is typically 30 nucleotides. The predetermined GC content range can be 15%-75%, or 40%-60%, or, 30%-70%. In the context of the present invention, the predetermined GC content range is typically 30%-70%.

A suitable GC content filter in the context of the invention may first analyze the first 30 nucleotides of the optimized nucleotide sequence, i.e., nucleotides 1 to 30 of the optimized nucleotide sequence. Analysis may comprise determining the number of nucleotides in the portion with are either G or C, and determining the GC content of the portion may comprise dividing the number of G or C nucleotides in the portion by the total number of nucleotides in the portion. The result of this analysis will provide a value describing the proportion of nucleotides in the portion that are G or C, and may be a percentage, for example 50%, or a decimal, for example 0.5. If the GC content of the first portion falls outside a predetermined GC content range, the optimized nucleotide sequence may be removed from the list of optimized nucleotide sequences.

If the GC content of the first portion falls inside the predetermined GC content range, the GC content filter may then analyze a second portion of the optimized nucleotide sequence. In this example, this may be the second 30 nucleotides, i.e., nucleotides 31 to 60, of the optimized nucleotide sequence. The portion analysis may be repeated for each portion until either: a portion is found having a GC content falling outside the predetermined GC content range, in which case the optimized nucleotide sequence may be removed from the list, or the whole optimized nucleotide sequence has been analyzed and no such portion has been found, in which case the GC content filter retains the optimized nucleotide sequence in the list and may move on to the next optimized nucleotide sequence in the list.

### Codon Adaptation Index (CAI)

The method further comprises determining a codon adaptation index of each of the optimized nucleotide sequences in the most recently updated list of optimized nucleotide sequences. The codon adaptation index of a sequence is a measure of codon usage bias and can be a value between 0 and 1. The most recently updated list of optimized nucleotide sequences is further updated by removing any nucleotide sequence if its codon adaptation index is less than or equal to a predetermined codon adaptation index threshold. The codon adaptation index threshold can 0.7, or 0.75, or 0.8, or 0.85, or 0.9. The inventors have found that optimized nucleotide sequences with a codon adaptation index equal to or greater than 0.8 deliver very high protein yield. Therefore in the context of the invention, the codon adaptation index threshold is typically 0.8.

A codon adaptation index may be calculated, for each optimized nucleotide sequence, in any way that would be apparent to a person skilled in the art, for example as described in “The codon adaptation index—a measure of directional synonymous codon usage bias, and its potential applications” (Sharp and Li, 1987. Nucleic Acids Research 15(3), p. 1281-1295); available online at https://www.ncbi.nlm.nih.gov/pmc/articles/PMC340524/.

Implementing a codon adaptation index calculation may include a method according to, or similar to, the following. For each amino acid in a sequence, a weight of each codon in a sequence may be represented by a parameter termed relative adaptiveness (wᵢ). Relative adaptiveness may be computed from a reference sequence set, as the ratio between the observed frequency of the codon fᵢ and the frequency of the most frequent synonymous codon fⱼ for that amino acid. The codon adaptation index of a sequence may then be calculated as the geometric mean of the weight associated to each codon over the length of the sequence (measured in codons). The reference sequence set used to calculate codon adaptation index may be the same reference sequence set from which a codon usage table used with methods of the invention is derived.

### Exemplary Optimized Nucleotide Sequences Encoding the Extracellular Domain of Human ACE2 Protein

Exemplary optimized nucleotide sequences encoding the extracellular domain of human ACE2 protein have been generated in accordance with the methods described herein, as shown in Table 3. The reference nucleotide sequence of SEQ TD NO: 1 has not been optimized in accordance with the methods of the invention, and consists of the protein-coding region of the mRNA encoding human ACE2 protein obtained from NCBI Genbank Reference Sequence NM_001371415.1.

TABLE 3
Exemplary optimized nucleotide sequences encoding the extracellular domain of
human ACE2 protein

| | SEQ ID | |
|---|---|---|
| NAME | NO: | SEQUENCE |
| Reference | 1 | AUGUCAAGCUCUUCCUGGCUCCUUCUCAGCCUUG |
| human ACE2 | | UUGCUGUAACUGCUGCUCAGUCCACCAUUGAGGA |
| mRNA protein | | ACAGGCCAAGACAUUUUUGGACAAGUUUAACCAC |
| coding | | GAAGCCGAAGACCUGUUCUAUCAAAGUUCACUUG |
| sequence | | CUUCUUGGAAUUAUAACACCAAUAUUACUGAAGA |
| (this sequence | | GAAUGUCCAAAACAUGAAUAAUGCUGGGGACAAA |
| consists of the | | UGGUCUGCCUUUUUAAAGGAACAGUCCACACUUG |
| protein-coding | | CCCAAAUGUAUCCACUACAAGAAAUUCAGAAUCU |
| region of the | | CACAGUCAAGCUUCAGCUGCAGGCUCUUCAGCAA |
| mRNA | | AAUGGGUCUUCAGUGCUCUCAGAAGACAAGAGCA |
| encoding | | AACGGUUGAACACAAUUCUAAAUACAAUGAGCAC |
| human ACE2 | | CAUCUACAGUACUGGAAAAGUUUGUAACCCAGAU |
| protein, and | | AAUCCACAAGAAUGCUUAUUACUUGAACCAGGUU |
| was obtained | | UGAAUGAAAUAAUGGCAAACAGUUUAGACUACAA |
| from NCBI | | UGAGAGGCUCUGGGCUUGGGAAAGCUGGAGAUCU |
| Genbank | | GAGGUCGGCAAGCAGCUGAGGCCAUUAUAUGAAG |
| Reference | | AGUAUGUGGUCUUGAAAAAUGAGAUGGCAAGAGC |
| Sequence | | AAAUCAUUAUGAGGACUAUGGGGAUUAUUGGAGA |
| NM_001371415.1) | | GGAGACUAUGAAGUAAAUGGGGUAGAUGGCUAUG |
| | | ACUACAGCCGCGGCCAGUUGAUUGAAGAUGUGGA |
| | | ACAUACCUUUGAAGAGAUUAAACCAUUAUAUGAA |
| | | CAUCUUCAUGCCUAUGUGAGGGCAAAGUUGAUGA |
| | | AUGCCUAUCCUUCCUAUAUCAGUCCAAUUGGAUG |
| | | CCUCCCUGCUCAUUUGCUUGGUGAUAUGUGGGGU |
| | | AGAUUUUGGACAAAUCUGUACUCUUUGACAGUUC |
| | | CCUUUGGACAGAAACCAAACAUAGAUGUUACUGA |
| | | UGCAAUGGUGGACCAGGCCUGGGAUGCACAGAGA |
| | | AUAUUCAAGGAGGCCGAGAAGUUCUUUGUAUCUG |
| | | UUGGUCUUCCUAAUAUGACUCAAGGAUUCUGGGA |
| | | AAAUUCCAUGCUAACGGACCCAGGAAAUGUUCAG |
| | | AAAGCAGUCUGCCAUCCCACAGCUUGGGACCUGG |
| | | GGAAGGGCGACUUCAGGAUCCUUAUGUGCACAAA |
| | | GGUGACAAUGGACGACUUCCUGACAGCUCAUCAU |
| | | GAGAUGGGGCAUAUCCAGUAUGAUAUGGCAUAUG |
| | | CUGCACAACCUUUUCUGCUAAGAAAUGGAGCUAA |
| | | UGAAGGAUUCCAUGAAGCUGUUGGGGAAAUCAUG |
| | | UCACUUUCUGCAGCCACACCUAAGCAUUUAAAAU |
| | | CCAUUGGUCUUCUGUCACCCGAUUUUCAAGAAGA |
| | | CAAUGAAACAGAAAUAAACUUCCUGCUCAAACAA |
| | | GCACUCACGAUUGUUGGGACUCUGCCAUUUACUU |
| | | ACAUGUUAGAGAAGUGGAGGUGGAUGGUCUUUAA |
| | | AGGGGAAAUUCCCAAAGACCAGUGGAUGAAAAAG |
| | | UGGUGGGAGAUGAAGCGAGAGAUAGUUGGGGUGG |
| | | UGGAACCUGUGCCCCAUGAUGAAACAUACUGUGA |
| | | CCCCGCAUCUCUGUUCCAUGUUUCUAAUGAUUAC |
| | | UCAUUCAUUCGAUAUUACACAAGGACCCUUUACC |
| | | AAUUCCAGUUUCAAGAAGCACUUUGUCAAGCAGC |
| | | UAAACAUGAAGGCCCUCUGCACAAAUGUGACAUC |
| | | UCAAACUCUACAGAAGCUGGACAGAAACUGUUCA |
| | | AUAUGCUGAGGCUUGGAAAAUCAGAACCCUGGAC |
| | | CCUAGCAUUGGAAAAUGUUGUAGGAGCAAAGAAC |
| | | AUGAAUGUAAGGCCACUGCUCAACUACUUUGAGC |
| | | CCUUAUUUACCUGGCUGAAAGACCAGAACAAGAA |
| | | UUCUUUUGUGGGAUGGAGUACCGACUGGAGUCCA |
| | | UAUGCAGACCAAAGCAUCAAAGUGAGGAUAAGCC |
| | | UAAAAUCAGCUCUUGGAGAUAAAGCAUAUGAAUG |
| | | GAACGACAAUGAAAUGUACCUGUUCCGAUCAUCU |
| | | GUUGCAUAUGCUAUGAGGCAGUACUUUUUAAAAG |
| | | UAAAAAAUCAGAUGAUUCUUUUUGGGGAGGAGGA |
| | | UGUGCGAGUGGCUAAUUUGAAACCAAGAAUCUCC |
| | | UUUAAUUUCUUUGUCACUGCACCUAAAAAUGUGU |
| | | CUGAUAUCAUUCCUAGAACUGAAGUUGAAAAGGC |
| | | CAUCAGGAUGUCCCGGAGCCGUAUCAAUGAUGCU |
| | | UUCCGUCUGAAUGACAACAGCCUAGAGUUUCUGG |
| | | GGAUACAGCCAACACUUGGACCUCCUAACCAGCCC |
| | | CCUGUUUCCAUAUGGCUGAUUGUUUUUGGAGUUG |
| | | UGAUGGGAGUGAUAGUGGUUGGCAUUGUCAUCCU |
| | | GAUCUUCACUGGGAUCAGAGAUCGGAAGAAGAAA |
| | | AAUAAAGCAAGAAGUGGAGAAAAUCCUUAUGCCU |
| | | CCAUCGAUAUUAGCAAAGGAGAAAAUAAUCCAGG |
| | | AUUCCAAAACACUGAUGAUGUUCAGACCUCCUUU |
| | | UAGAAAAAUCUAUGUUUUUCCUCUUGAGGUGAUU |
| | | UUGUUGUAUGUAAAUGUUAAUUUCAUGGUAUAGA |
| | | AAAUAUAAGAUGAUAAAGAUAUCAUUAAAUGUCA |
| | | AAACUAUGACUCUGUUCAGAAAAAAAAUUGUCCA |
| | | AAGACAACAUGGCCAAGGAGAGAGCAUCUUCAUU |
| | | GACAUUGCUUUCAGUAUUUAUUUCUGUCUCUGGA |
| | | UUUGACUUCUGUUCUGUUUCUUAAUAAGGAUUUU |
| | | GUAUUAGAGUAUAUUAGGGAAAGUGUGUAUUUGG |
| | | UCUCACAGGCUGUUCAGGGAUAAUCUAAAUGUAA |
| | | AUGUCUGUUGAAUUUCUGAAGUUGAAAACAAGGA |
| | | UAUAUCAUUGGAGCAAGUGUUGGAUCUUGUAUGG |
| | | AAUAUGGAUGGAUCACUUGUAAGGACAGUGCCUG |
| | | GGAACUGGUGUAGCUGCAAGGAUUGAGAAUGGCA |
| | | UGCAUUAGCUCACUUUCAUUUAAUCCAUUGUCAA |
| | | GGAUGACAUGCUUUCUUCACAGUAACUCAGUUCA |
| | | AGUACUAUGGUGAUUUGCCUACAGUGAUGUUUGG |
| | | AAUCGAUCAUGCUUUCUUCAAGGUGACAGGUCUA |
| | | AAGAGAGAAGAAUCCAGGGAACAGGUAGAGGACA |
| | | UUGCUUUUUCACUUCCAAGGUGCUUGAUCAACAU |
| | | CUCCCUGACAACACAAAACUAGAGCCAGGGGCCUC |
| | | CGUGAACUCCCAGAGCAUGCCUGAUAGAAACUCA |
| | | UUUCUACUGUUCUCUAACUGUGGAGUGAAUGGAA |
| | | AUUCCAACUGUAUGUUCACCCUCUGAAGUGGGUA |
| | | CCCAGUCUCUUAAAUCUUUUGUAUUUGCUCACAG |
| | | UGUUUGAGCAGUGCUGA |
| RNA sequence | 2 | AUGUCUUCCUCUUCUUGGCUGCUGCUGAGCCUGG |
| encoding | | UGGCCGUCACAGCCGCUCAGUCCACAAUCGAGGA |
| hsACE2 | | ACAGGCUAAAACUUUCCUGGACAAAUUCAACCAC |
| | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCUGA |
| RNA sequence | 3 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCGGCGGAGGGGGAAGCCCGAAGUCUUGCG |
| | | AUAAAACCCACACAUGUCCUCCAUGUCCAGCACCC |
| | | GAGUUACUGGGCGGCCCUAGCGUGUUCCUGUUUC |
| | | CACCUAAGCCAAAGGAUACACUGAUGAUCUCACG |
| | | GACACCUGAGGUGACUUGUGUUGUGGUGGACGUG |
| | | UCUCAUGAGGACCCUGAGGUGAAAUUCAACUGGU |
| | | ACGUGGACGGCGUGGAGGUGCACAACGCCAAAAC |
| | | CAAGCCCAGGGAGGAGCAGUACAACUCCACCUAC |
| | | AGGGUGGUGUCUGUGUUGACAGUGCUGCAUCAGG |
| | | ACUGGCUGAACGGGAAGGAGUAUAAGUGUAAGGU |
| | | GUCCAAUAAGGCCCUCCCCGCCCCUAUCGAAAAGA |
| | | CAAUCAGCAAGGCUAAGGGACAGCCCAGGGAGCC |
| | | ACAGGUGUAUACUCUGCCACCUUCACGGGAUGAA |
| | | CUGACCAAGAAUCAGGUGUCUCUGACCUGCCUGG |
| | | UGAAGGGGUUUUACCCAUCCGACAUCGCCGUCGA |
| | | AUGGGAAAGCAAUGGCCAACCCGAGAACAAUUAU |
| | | AAGACAACCCCACCAGUUCUGGACUCCGAUGGAU |
| | | CCUUCUUCCUGUACAGCAAGCUGACUGUGGAUAA |
| | | GUCUCGCUGGCAGCAGGGAAACGUGUUCUCUUGC |
| | | UCCGUGAUGCACGAGGCUCUGCACAAUCAUUACA |
| | | CACAGAAAUCCCUGUCUCUGUCCCCAGGAAAGUG |
| | | A |
| RNA sequence | 4 | AUGUCUUCCUCUUCUUGGCUGCUGCUGAGCCUGG |
| encoding | | UGGCCGUCACAGCCGCUCAGUCCACAAUCGAGGA |
| hsACE2 N90D | | ACAGGCUAAAACUUUCCUGGACAAAUUCAACCAC |
| R273A | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGGACCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCG |
| | | CCUUUUGGACCAAUCUCUACUCUCUGACUGUCCCC |
| | | UUUGGCCAGAAGCCUAACAUCGACGUGACAGAUG |
| | | CAAUGGUGGACCAGGCAUGGGACGCCCAGAGGAU |
| | | CUUCAAGGAAGCAGAGAAAUUCUUCGUGUCCGUG |
| | | GGACUGCCAAAUAUGACCCAGGGCUUUUGGGAGA |
| | | AUAGCAUGCUGACCGACCCAGGGAACGUGCAGAA |
| | | GGCUGUGUGUCACCCUACAGCCUGGGAUCUGGGG |
| | | AAGGGCGACUUCAGGAUCCUGAUGUGUACAAAGG |
| | | UUACAAUGGACGAUUUCCUGACAGCCCAUCACGA |
| | | GAUGGGCCAUAUCCAGUACGACAUGGCCUAUGCU |
| | | GCUCAGCCUUUUCUGCUGAGGAAUGGAGCAAAUG |
| | | AGGGGUUCCAUGAGGCCGUGGGAGAGAUUAUGUC |
| | | CCUGUCUGCAGCUACCCCUAAACACCUGAAAAGCA |
| | | UCGGCCUGCUGUCACCAGACUUCCAGGAGGACAA |
| | | CGAGACAGAGAUCAACUUUCUGCUGAAGCAGGCA |
| | | CUGACAAUCGUGGGGACACUGCCUUUCACCUAUA |
| | | UGCUGGAAAAGUGGAGGUGGAUGGUGUUCAAGGG |
| | | CGAAAUUCCUAAGGACCAGUGGAUGAAGAAAUGG |
| | | UGGGAGAUGAAAAGGGAGAUUGUGGGCGUGGUGG |
| | | AGCCUGUGCCUCACGAUGAGACUUAUUGUGACCC |
| | | CGCCAGCCUGUUUCACGUCUCCAACGACUACUCCU |
| | | UCAUCAGAUAUUACACAAGAACUCUGUACCAGUU |
| | | CCAGUUCCAGGAGGCCCUGUGUCAGGCCGCCAAAC |
| | | ACGAGGGGCCACUGCAUAAAUGCGAUAUUAGCAA |
| | | CAGCACCGAGGCCGGCCAGAAGCUCUUUAAUAUG |
| | | CUGAGGCUGGGCAAGUCCGAGCCUUGGACUCUAG |
| | | CCCUGGAGAACGUGGUGGGAGCAAAGAACAUGAA |
| | | UGUGAGACCACUGCUGAACUACUUCGAGCCCCUG |
| | | UUUACCUGGCUGAAAGACCAGAACAAAAACUCUU |
| | | UUGUGGGCUGGAGCACCGAUUGGUCUCCAUACGC |
| | | AGACCAGAGCAUCAAAGUCCGCAUCUCCCUGAAA |
| | | UCAGCCCUGGGGGAUAAGGCCUAUGAGUGGAAUG |
| | | ACAAUGAAAUGUACCUCUUUCGGUCCAGCGUGGC |
| | | AUAUGCUAUGCGGCAGUACUUCCUGAAGGUCAAG |
| | | AACCAGAUGAUCCUGUUUGGCGAGGAAGACGUGA |
| | | GGGUUGCCAAUCUGAAGCCCCGCAUUUCUUUUAA |
| | | CUUUUUCGUCACUGCCCCCAAGAACGUGAGCGAC |
| | | AUCAUUCCAAGGACCGAAGUGGAAAAGGCUAUCA |
| | | GAAUGUCUCGGUCCAGGAUCAACGACGCCUUCAG |
| | | GUUGAACGACAACAGCCUGGAGUUUCUGGGGAUU |
| | | CAGCCUACACUGGGGCCUCCCAAUCAGCCACCCGU |
| | | GAGCUGA |
| RNA sequence | 5 | AUGUCUUCCUCUUCUUGGCUGCUGCUGAGCCUGG |
| encoding | | UGGCCGUCACAGCCGCUCAGUCCACAAUCGAGGA |
| hsACE2 N90D | | ACAGGCUAAAACUUUCCUGGACAAAUUCAACCAC |
| T92A | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGGACCU |
| | | GGCCGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCUGA |
| RNA sequence | 6 | AUGUCUUCCUCUUCUUGGCUGCUGCUGAGCCUGG |
| encoding | | UGGCCGUCACAGCCGCUCAGUCCACAAUCGAGGA |
| hsACE2 | | ACAGGCUAAAACUUUCCUGGACAAAUUCAACCAC |
| H374N H378N | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUAACG |
| | | AGAUGGGCAAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCUGA |
| RNA sequence | 7 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc-GS-KLKL | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCGGCGGAGGGGGAAGCCCGAAGUCUUGCG |
| | | AUAAAACCCACACAUGUCCUCCAUGUCCAGCACCC |
| | | GAGUUACUGGGCGGCCCUAGCGUGUUCCUGUUUC |
| | | CACCUAAGCCAAAGGAUACACUGAUGAUCUCACG |
| | | GACACCUGAGGUGACUUGUGUUGUGGUGGACGUG |
| | | UCUCAUGAGGACCCUGAGGUGAAAUUCAACUGGU |
| | | ACGUGGACGGCGUGGAGGUGCACAACGCCAAAAC |
| | | CAAGCCCAGGGAGGAGCAGUACAACUCCACCUAC |
| | | AGGGUGGUGUCUGUGUUGACAGUGCUGCAUCAGG |
| | | ACUGGCUGAACGGGAAGGAGUAUAAGUGUAAGGU |
| | | GUCCAAUAAGGCCCUCCCCGCCCCUAUCGAAAAGA |
| | | CAAUCAGCAAGGCUAAGGGACAGCCCAGGGAGCC |
| | | ACAGGUGUAUACUCUGCCACCUUCACGGGAUGAA |
| | | CUGACCAAGAAUCAGGUGUCUCUGACCUGCCUGG |
| | | UGAAGGGGUUUUACCCAUCCGACAUCGCCGUCGA |
| | | AUGGGAAAGCAAUGGCCAACCCGAGAACAAUUAU |
| | | AAGACAACCCCACCAGUUCUGGACUCCGAUGGAU |
| | | CCUUCUUCCUGUACAGCAAGCUGACUGUGGAUAA |
| | | GUCUCGCUGGCAGCAGGGAAACGUGUUCUCUUGC |
| | | UCCGUGAUGCACGAGGCUCUGCACAAUCAUUACA |
| | | CACAGAAAUCCCUGUCUCUGUCCCCAGGAAAGGG |
| | | CGGAGGGGGAAGCCAGAGGAACCCAAAGCUGAAA |
| | | CUGAUUCGCAGACACCCUACCCUGCGCAUCCCACC |
| | | CAUUUGA |
| RNA sequence | 8 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc-GS-RLRL | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCGGCGGAGGGGGAAGCCCGAAGUCUUGCG |
| | | AUAAAACCCACACAUGUCCUCCAUGUCCAGCACCC |
| | | GAGUUACUGGGCGGCCCUAGCGUGUUCCUGUUUC |
| | | CACCUAAGCCAAAGGAUACACUGAUGAUCUCACG |
| | | GACACCUGAGGUGACUUGUGUUGUGGUGGACGUG |
| | | UCUCAUGAGGACCCUGAGGUGAAAUUCAACUGGU |
| | | ACGUGGACGGCGUGGAGGUGCACAACGCCAAAAC |
| | | CAAGCCCAGGGAGGAGCAGUACAACUCCACCUAC |
| | | AGGGUGGUGUCUGUGUUGACAGUGCUGCAUCAGG |
| | | ACUGGCUGAACGGGAAGGAGUAUAAGUGUAAGGU |
| | | GUCCAAUAAGGCCCUCCCCGCCCCUAUCGAAAAGA |
| | | CAAUCAGCAAGGCUAAGGGACAGCCCAGGGAGCC |
| | | ACAGGUGUAUACUCUGCCACCUUCACGGGAUGAA |
| | | CUGACCAAGAAUCAGGUGUCUCUGACCUGCCUGG |
| | | UGAAGGGGUUUUACCCAUCCGACAUCGCCGUCGA |
| | | AUGGGAAAGCAAUGGCCAACCCGAGAACAAUUAU |
| | | AAGACAACCCCACCAGUUCUGGACUCCGAUGGAU |
| | | CCUUCUUCCUGUACAGCAAGCUGACUGUGGAUAA |
| | | GUCUCGCUGGCAGCAGGGAAACGUGUUCUCUUGC |
| | | UCCGUGAUGCACGAGGCUCUGCACAAUCAUUACA |
| | | CACAGAAAUCCCUGUCUCUGUCCCCAGGAAAGGG |
| | | CGGAGGGGGAAGCCAGCGCAAUCCUCGCCUGCGG |
| | | CUGAUCCGGAGACAUCCUACACUGAGAAUUCCCCC |
| | | AAUUUGA |
| RNA sequence | 9 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc-GS-J chain | | GAAGCAGAGGACCUCUUCUAUCAGUCUAGCCUGG |
| | | CCUCCUGGAACUACAACACCAACAUUACUGAGGA |
| | | GAACGUGCAGAACAUGAACAACGCCGGAGACAAG |
| | | UGGUCCGCCUUCCUGAAGGAGCAGUCUACGCUGG |
| | | CACAGAUGUACCCACUGCAGGAGAUUCAGAAUCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCGGCGGAGGGGGAAGCCCGAAGUCUUGCG |
| | | AUAAAACCCACACAUGUCCUCCAUGUCCAGCACCC |
| | | GAGUUACUGGGCGGCCCUAGCGUGUUCCUGUUUC |
| | | CACCUAAGCCAAAGGAUACACUGAUGAUCUCACG |
| | | GACACCUGAGGUGACUUGUGUUGUGGUGGACGUG |
| | | UCUCAUGAGGACCCUGAGGUGAAAUUCAACUGGU |
| | | ACGUGGACGGCGUGGAGGUGCACAACGCCAAAAC |
| | | CAAGCCCAGGGAGGAGCAGUACAACUCCACCUAC |
| | | AGGGUGGUGUCUGUGUUGACAGUGCUGCAUCAGG |
| | | ACUGGCUGAACGGGAAGGAGUAUAAGUGUAAGGU |
| | | GUCCAAUAAGGCCCUCCCCGCCCCUAUCGAAAAGA |
| | | CAAUCAGCAAGGCUAAGGGACAGCCCAGGGAGCC |
| | | ACAGGUGUAUACUCUGCCACCUUCACGGGAUGAA |
| | | CUGACCAAGAAUCAGGUGUCUCUGACCUGCCUGG |
| | | UGAAGGGGUUUUACCCAUCCGACAUCGCCGUCGA |
| | | AUGGGAAAGCAAUGGCCAACCCGAGAACAAUUAU |
| | | AAGACAACCCCACCAGUUCUGGACUCCGAUGGAU |
| | | CCUUCUUCCUGUACAGCAAGCUGACUGUGGAUAA |
| | | GUCUCGCUGGCAGCAGGGAAACGUGUUCUCUUGC |
| | | UCCGUGAUGCACGAGGCUCUGCACAAUCAUUACA |
| | | CACAGAAAUCCCUGUCUCUGUCCCCAGGAAAGGG |
| | | CGGAGGGGGAAGCCAGGAGGACGAGCGCAUCGUU |
| | | CUGGUGGAUAACAAGUGCAAGUCCGCUAGGAUCA |
| | | CUUCCAGGAUCAUUAGGUCCUCCGAGGACCCAAA |
| | | UGAAGAUAUUGUGGAGCGGAACAUUCGGAUUAUU |
| | | GUUCCCCUGAACAAUCGGGAGAAUAUCAGCGACC |
| | | CUACCUCCCCACUCAGAACCAGAUUUGUGUAUCAC |
| | | CUGAGUGACCUGUCCAAGAAGUGCGAUCCCACCG |
| | | AAGUGGAGCUGGAUAAUCAGAUUGUGACCGCUAC |
| | | UCAGUCUAACAUCUGCGACGAAGAUUCCGCCACA |
| | | GAAACAUGCUAUACAUACGAUAGAAACAAGUGUU |
| | | ACACUGCCGUGGUCCCUCUGGUGUACGGCGGAGA |
| | | AACCAAGAUGGUGGAGACCGCCCUGACCCCAGAC |
| | | GCAUGUUACCCAGACUGA |
| RNA sequence | 32 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc, wherein the | | GAAGCAGAGGACCUCUUCUAUCAGUCUAGCCUGG |
| hsACE2 is | | CCUCCUGGAACUACAACACCAACAUUACUGAGGA |
| hsACE2 N90D | | GAACGUGCAGAACAUGAACAACGCCGGAGACAAG |
| R273A | | UGGUCCGCCUUCCUGAAGGAGCAGUCUACGCUGG |
| | | CACAGAUGUACCCACUGCAGGAGAUUCAGGACCU |
| | | GACAGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCG |
| | | CCUUUUGGACCAAUCUCUACUCUCUGACUGUCCCC |
| | | UUUGGCCAGAAGCCUAACAUCGACGUGACAGAUG |
| | | CAAUGGUGGACCAGGCAUGGGACGCCCAGAGGAU |
| | | CUUCAAGGAAGCAGAGAAAUUCUUCGUGUCCGUG |
| | | GGACUGCCAAAUAUGACCCAGGGCUUUUGGGAGA |
| | | AUAGCAUGCUGACCGACCCAGGGAACGUGCAGAA |
| | | GGCUGUGUGUCACCCUACAGCCUGGGAUCUGGGG |
| | | AAGGGCGACUUCAGGAUCCUGAUGUGUACAAAGG |
| | | UUACAAUGGACGAUUUCCUGACAGCCCAUCACGA |
| | | GAUGGGCCAUAUCCAGUACGACAUGGCCUAUGCU |
| | | GCUCAGCCUUUUCUGCUGAGGAAUGGAGCAAAUG |
| | | AGGGGUUCCAUGAGGCCGUGGGAGAGAUUAUGUC |
| | | CCUGUCUGCAGCUACCCCUAAACACCUGAAAAGCA |
| | | UCGGCCUGCUGUCACCAGACUUCCAGGAGGACAA |
| | | CGAGACAGAGAUCAACUUUCUGCUGAAGCAGGCA |
| | | CUGACAAUCGUGGGGACACUGCCUUUCACCUAUA |
| | | UGCUGGAAAAGUGGAGGUGGAUGGUGUUCAAGGG |
| | | CGAAAUUCCUAAGGACCAGUGGAUGAAGAAAUGG |
| | | UGGGAGAUGAAAAGGGAGAUUGUGGGCGUGGUGG |
| | | AGCCUGUGCCUCACGAUGAGACUUAUUGUGACCC |
| | | CGCCAGCCUGUUUCACGUCUCCAACGACUACUCCU |
| | | UCAUCAGAUAUUACACAAGAACUCUGUACCAGUU |
| | | CCAGUUCCAGGAGGCCCUGUGUCAGGCCGCCAAAC |
| | | ACGAGGGGCCACUGCAUAAAUGCGAUAUUAGCAA |
| | | CAGCACCGAGGCCGGCCAGAAGCUCUUUAAUAUG |
| | | CUGAGGCUGGGCAAGUCCGAGCCUUGGACUCUAG |
| | | CCCUGGAGAACGUGGUGGGAGCAAAGAACAUGAA |
| | | UGUGAGACCACUGCUGAACUACUUCGAGCCCCUG |
| | | UUUACCUGGCUGAAAGACCAGAACAAAAACUCUU |
| | | UUGUGGGCUGGAGCACCGAUUGGUCUCCAUACGC |
| | | AGACCAGAGCAUCAAAGUCCGCAUCUCCCUGAAA |
| | | UCAGCCCUGGGGGAUAAGGCCUAUGAGUGGAAUG |
| | | ACAAUGAAAUGUACCUCUUUCGGUCCAGCGUGGC |
| | | AUAUGCUAUGCGGCAGUACUUCCUGAAGGUCAAG |
| | | AACCAGAUGAUCCUGUUUGGCGAGGAAGACGUGA |
| | | GGGUUGCCAAUCUGAAGCCCCGCAUUUCUUUUAA |
| | | CUUUUUCGUCACUGCCCCCAAGAACGUGAGCGAC |
| | | AUCAUUCCAAGGACCGAAGUGGAAAAGGCUAUCA |
| | | GAAUGUCUCGGUCCAGGAUCAACGACGCCUUCAG |
| | | GUUGAACGACAACAGCCUGGAGUUUCUGGGGAUU |
| | | CAGCCUACACUGGGGCCUCCCAAUCAGCCACCCGU |
| | | GAGCUGAGGCGGAGGGGGAAGCCCGAAGUCUUGC |
| | | GAUAAAACCCACACAUGUCCUCCAUGUCCAGCACC |
| | | CGAGUUACUGGGCGGCCCUAGCGUGUUCCUGUUU |
| | | CCACCUAAGCCAAAGGAUACACUGAUGAUCUCAC |
| | | GGACACCUGAGGUGACUUGUGUUGUGGUGGACGU |
| | | GUCUCAUGAGGACCCUGAGGUGAAAUUCAACUGG |
| | | UACGUGGACGGCGUGGAGGUGCACAACGCCAAAA |
| | | CCAAGCCCAGGGAGGAGCAGUACAACUCCACCUAC |
| | | AGGGUGGUGUCUGUGUUGACAGUGCUGCAUCAGG |
| | | ACUGGCUGAACGGGAAGGAGUAUAAGUGUAAGGU |
| | | GUCCAAUAAGGCCCUCCCCGCCCCUAUCGAAAAGA |
| | | CAAUCAGCAAGGCUAAGGGACAGCCCAGGGAGCC |
| | | ACAGGUGUAUACUCUGCCACCUUCACGGGAUGAA |
| | | CUGACCAAGAAUCAGGUGUCUCUGACCUGCCUGG |
| | | UGAAGGGGUUUUACCCAUCCGACAUCGCCGUCGA |
| | | AUGGGAAAGCAAUGGCCAACCCGAGAACAAUUAU |
| | | AAGACAACCCCACCAGUUCUGGACUCCGAUGGAU |
| | | CCUUCUUCCUGUACAGCAAGCUGACUGUGGAUAA |
| | | GUCUCGCUGGCAGCAGGGAAACGUGUUCUCUUGC |
| | | UCCGUGAUGCACGAGGCUCUGCACAAUCAUUACA |
| | | CACAGAAAUCCCUGUCUCUGUCCCCAGGAAAGUG |
| | | ACCGUGAGCUGA |
| RNA sequence | 33 | AUGUCUUCCUCUUCUUGGCUGCUGCUGAGCCUGG |
| encoding | | UGGCCGUCACAGCCGCUCAGUCCACAAUCGAGGA |
| hsACE2-GS- | | ACAGGCUAAAACUUUCCUGGACAAAUUCAACCAC |
| Fc, wherein the | | GAAGCAGAGGACCUCUUCUAUCAGUCUAGCCUGG |
| hsACE2 is | | CCUCCUGGAACUACAACACCAACAUUACUGAGGA |
| hsACE2 N90D | | GAACGUGCAGAACAUGAACAACGCCGGAGACAAG |
| T92A | | UGGUCCGCCUUCCUGAAGGAGCAGUCUACGCUGG |
| | | CACAGAUGUACCCACUGCAGGAGAUUCAGGACCU |
| | | GGCCGUGAAGCUGCAGCUUCAGGCUCUGCAGCAG |
| | | AACGGGUCUAGUGUGCUGUCUGAGGACAAAUCUA |
| | | AGAGGUUGAACACCAUUCUGAAUACAAUGUCCAC |
| | | AAUCUAUAGCACCGGCAAGGUGUGCAAUCCUGAU |
| | | AAUCCUCAGGAGUGCCUGCUGCUGGAGCCAGGAC |
| | | UGAAUGAAAUUAUGGCUAACUCCCUGGAUUACAA |
| | | CGAAAGACUGUGGGCCUGGGAGAGCUGGAGGUCC |
| | | GAGGUGGGAAAGCAGCUGAGACCUCUGUAUGAGG |
| | | AGUAUGUGGUCCUGAAGAAUGAGAUGGCCCGCGC |
| | | CAACCACUACGAAGACUAUGGAGACUACUGGAGA |
| | | GGAGACUAUGAGGUGAACGGAGUGGAUGGAUAUG |
| | | ACUACAGCAGGGGCCAGCUGAUCGAAGAUGUGGA |
| | | ACAUACCUUCGAGGAGAUCAAGCCCCUGUAUGAG |
| | | CAUCUGCACGCAUACGUCAGGGCAAAGCUGAUGA |
| | | ACGCCUAUCCCUCCUACAUCAGCCCAAUCGGGUGU |
| | | CUGCCCGCUCACCUCCUGGGGGAUAUGUGGGGCA |
| | | GGUUUUGGACCAAUCUCUACUCUCUGACUGUCCC |
| | | CUUUGGCCAGAAGCCUAACAUCGACGUGACAGAU |
| | | GCAAUGGUGGACCAGGCAUGGGACGCCCAGAGGA |
| | | UCUUCAAGGAAGCAGAGAAAUUCUUCGUGUCCGU |
| | | GGGACUGCCAAAUAUGACCCAGGGCUUUUGGGAG |
| | | AAUAGCAUGCUGACCGACCCAGGGAACGUGCAGA |
| | | AGGCUGUGUGUCACCCUACAGCCUGGGAUCUGGG |
| | | GAAGGGCGACUUCAGGAUCCUGAUGUGUACAAAG |
| | | GUUACAAUGGACGAUUUCCUGACAGCCCAUCACG |
| | | AGAUGGGCCAUAUCCAGUACGACAUGGCCUAUGC |
| | | UGCUCAGCCUUUUCUGCUGAGGAAUGGAGCAAAU |
| | | GAGGGGUUCCAUGAGGCCGUGGGAGAGAUUAUGU |
| | | CCCUGUCUGCAGCUACCCCUAAACACCUGAAAAGC |
| | | AUCGGCCUGCUGUCACCAGACUUCCAGGAGGACA |
| | | ACGAGACAGAGAUCAACUUUCUGCUGAAGCAGGC |
| | | ACUGACAAUCGUGGGGACACUGCCUUUCACCUAU |
| | | AUGCUGGAAAAGUGGAGGUGGAUGGUGUUCAAGG |
| | | GCGAAAUUCCUAAGGACCAGUGGAUGAAGAAAUG |
| | | GUGGGAGAUGAAAAGGGAGAUUGUGGGCGUGGUG |
| | | GAGCCUGUGCCUCACGAUGAGACUUAUUGUGACC |
| | | CCGCCAGCCUGUUUCACGUCUCCAACGACUACUCC |
| | | UUCAUCAGAUAUUACACAAGAACUCUGUACCAGU |
| | | UCCAGUUCCAGGAGGCCCUGUGUCAGGCCGCCAA |
| | | ACACGAGGGGCCACUGCAUAAAUGCGAUAUUAGC |
| | | AACAGCACCGAGGCCGGCCAGAAGCUCUUUAAUA |
| | | UGCUGAGGCUGGGCAAGUCCGAGCCUUGGACUCU |
| | | AGCCCUGGAGAACGUGGUGGGAGCAAAGAACAUG |
| | | AAUGUGAGACCACUGCUGAACUACUUCGAGCCCC |
| | | UGUUUACCUGGCUGAAAGACCAGAACAAAAACUC |
| | | UUUUGUGGGCUGGAGCACCGAUUGGUCUCCAUAC |
| | | GCAGACCAGAGCAUCAAAGUCCGCAUCUCCCUGA |
| | | AAUCAGCCCUGGGGGAUAAGGCCUAUGAGUGGAA |
| | | UGACAAUGAAAUGUACCUCUUUCGGUCCAGCGUG |
| | | GCAUAUGCUAUGCGGCAGUACUUCCUGAAGGUCA |
| | | AGAACCAGAUGAUCCUGUUUGGCGAGGAAGACGU |
| | | GAGGGUUGCCAAUCUGAAGCCCCGCAUUUCUUUU |
| | | AACUUUUUCGUCACUGCCCCCAAGAACGUGAGCG |
| | | ACAUCAUUCCAAGGACCGAAGUGGAAAAGGCUAU |
| | | CAGAAUGUCUCGGUCCAGGAUCAACGACGCCUUC |
| | | AGGUUGAACGACAACAGCCUGGAGUUUCUGGGGA |
| | | UUCAGCCUACACUGGGGCCUCCCAAUCAGCCACCC |
| | | GUGAGCUGAGGCGGAGGGGGAAGCCCGAAGUCUU |
| | | GCGAUAAAACCCACACAUGUCCUCCAUGUCCAGCA |
| | | CCCGAGUUACUGGGCGGCCCUAGCGUGUUCCUGU |
| | | UUCCACCUAAGCCAAAGGAUACACUGAUGAUCUC |
| | | ACGGACACCUGAGGUGACUUGUGUUGUGGUGGAC |
| | | GUGUCUCAUGAGGACCCUGAGGUGAAAUUCAACU |
| | | GGUACGUGGACGGCGUGGAGGUGCACAACGCCAA |
| | | AACCAAGCCCAGGGAGGAGCAGUACAACUCCACC |
| | | UACAGGGUGGUGUCUGUGUUGACAGUGCUGCAUC |
| | | AGGACUGGCUGAACGGGAAGGAGUAUAAGUGUAA |
| | | GGUGUCCAAUAAGGCCCUCCCCGCCCCUAUCGAAA |
| | | AGACAAUCAGCAAGGCUAAGGGACAGCCCAGGGA |
| | | GCCACAGGUGUAUACUCUGCCACCUUCACGGGAU |
| | | GAACUGACCAAGAAUCAGGUGUCUCUGACCUGCC |
| | | UGGUGAAGGGGUUUUACCCAUCCGACAUCGCCGU |
| | | CGAAUGGGAAAGCAAUGGCCAACCCGAGAACAAU |
| | | UAUAAGACAACCCCACCAGUUCUGGACUCCGAUG |
| | | GAUCCUUCUUCCUGUACAGCAAGCUGACUGUGGA |
| | | UAAGUCUCGCUGGCAGCAGGGAAACGUGUUCUCU |
| | | UGCUCCGUGAUGCACGAGGCUCUGCACAAUCAUU |
| | | ACACACAGAAAUCCCUGUCUCUGUCCCCAGGAAA |
| | | GUGACCGUGAGCUGA |

### Exemplary Optimized Nucleotide Sequences Encoding Immunoglobulin Fe Region, Linker Sequence, and Localization Sequences

Exemplary optimized nucleotide sequences encoding an exemplary immunoglobulin Fc region, exemplary linker sequence, and exemplary localization sequences have been generated in accordance with the methods described herein, as shown in Table 4.

TABLE 4
Exemplary optimized nucleotide sequences encoding exemplary immunoglobulin
Fc region, linker sequence, and localization sequences

| NAME | SEQ ID | |
|---|---|---|
| | NO: | SEQUENCE |
| RNA sequence | 22 | CCGAAGUCUUGCGAUAAAACCCACACAUGUCCUCC |
| encoding | | AUGUCCAGCACCCGAGUUACUGGGCGGCCCUAGC |
| immunoglobulin | | GUGUUCCUGUUUCCACCUAAGCCAAAGGAUACAC |
| Fc region from | | UGAUGAUCUCACGGACACCUGAGGUGACUUGUGU |
| human IgG1 | | UGUGGUGGACGUGUCUCAUGAGGACCCUGAGGUG |
| | | AAAUUCAACUGGUACGUGGACGGCGUGGAGGUGC |
| | | ACAACGCCAAAACCAAGCCCAGGGAGGAGCAGUA |
| | | CAACUCCACCUACAGGGUGGUGUCUGUGUUGACA |
| | | GUGCUGCAUCAGGACUGGCUGAACGGGAAGGAGU |
| | | AUAAGUGUAAGGUGUCCAAUAAGGCCCUCCCCGC |
| | | CCCUAUCGAAAAGACAAUCAGCAAGGCUAAGGGA |
| | | CAGCCCAGGGAGCCACAGGUGUAUACUCUGCCACC |
| | | UUCACGGGAUGAACUGACCAAGAAUCAGGUGUCU |
| | | CUGACCUGCCUGGUGAAGGGGUUUUACCCAUCCG |
| | | ACAUCGCCGUCGAAUGGGAAAGCAAUGGCCAACC |
| | | CGAGAACAAUUAUAAGACAACCCCACCAGUUCUG |
| | | GACUCCGAUGGAUCCUUCUUCCUGUACAGCAAGC |
| | | UGACUGUGGAUAAGUCUCGCUGGCAGCAGGGAAA |
| | | CGUGUUCUCUUGCUCCGUGAUGCACGAGGCUCUG |
| | | CACAAUCAUUACACACAGAAAUCCCUGUCUCUGU |
| | | CCCCAGGAAAG |
| RNA sequence | 23 | GGCGGAGGGGGAAGC |
| encoding GS | | |
| linker | | |
| RNA sequence | 24 | CAGAGGAACCCAAAGCUGAAACUGAUUCGCAGAC |
| encoding KLKL | | ACCCUACCCUGCGCAUCCCACCCAUU |
| localization | | |
| sequence | | |
| RNA sequence | 25 | CAGCGCAAUCCUCGCCUGCGGCUGAUCCGGAGACA |
| encoding RLRL | | UCCUACACUGAGAAUUCCCCCAAUU |
| localization | | |
| sequence | | |
| RNA sequence | 26 | CAGGAGGACGAGCGCAUCGUUCUGGUGGAUAACA |
| encoding | | AGUGCAAGUCCGCUAGGAUCACUUCCAGGAUCAU |
| immunoglobulin J | | UAGGUCCUCCGAGGACCCAAAUGAAGAUAUUGUG |
| chain | | GAGCGGAACAUUCGGAUUAUUGUUCCCCUGAACA |
| | | AUCGGGAGAAUAUCAGCGACCCUACCUCCCCACUC |
| | | AGAACCAGAUUUGUGUAUCACCUGAGUGACCUGU |
| | | CCAAGAAGUGCGAUCCCACCGAAGUGGAGCUGGA |
| | | UAAUCAGAUUGUGACCGCUACUCAGUCUAACAUC |
| | | UGCGACGAAGAUUCCGCCACAGAAACAUGCUAUA |
| | | CAUACGAUAGAAACAAGUGUUACACUGCCGUGGU |
| | | CCCUCUGGUGUACGGCGGAGAAACCAAGAUGGUG |
| | | GAGACCGCCCUGACCCCAGACGCAUGUUACCCAGA |
| | | C |

mRNAs
Structural Elements of mRNTAs

A typical mRNA in accordance with the invention comprises a 5′ cap, a 5′ untranslated region (5′ UTR), a protein-coding region, a 3′ untranslated region (3′ UTR), and a 3′ tail.

### 5′ Cap

In a specific embodiment, the mRNA of the invention comprises a 5′ cap with the following structure:

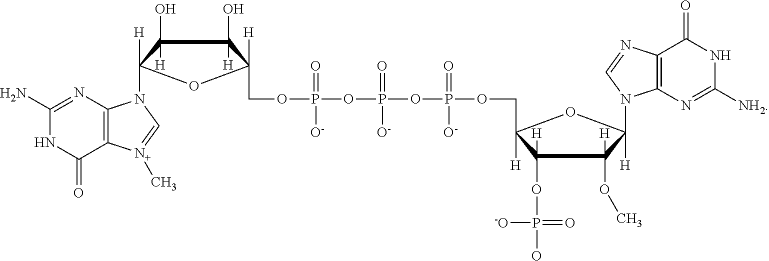

Typically, a 5′ cap and/or a 3′ tail may be added after mRNA synthesis. The presence of the cap is important in providing resistance to nucleases found in most eukaryotic cells. The presence of a “tail” serves to protect the mRNA from exonuclease degradation. Alternatively, the 5′ cap and/or a 3′ tail sequences are included in the DNA template sequences used in in vitro transcription reaction.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

### 3′ Tail

In one specific embodiment, the tail structure of the mRNA comprises a poly(A) tail. In another specific embodiment, the tail structure of the mRNA comprises a poly(C) tail. In some embodiments, the tail structure comprises at least 50 adenosine or cytosine nucleotides. In a typical embodiment, the tail structure is approximately 100-500 nucleotides in length.

A poly(A) or poly(C) tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, respectively. In some embodiments, a tail structure includes combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### 5′ UTRs and 3′ UTRs

In some embodiments, an mRNA comprising an optimized nucleotide sequence encoding a polypeptide comprising the extracellular domain of human ACE2 protein also contains 5′ and 3′ untranslated region (UTR) sequences. In some embodiments, the mRNA comprises a 5′ untranslated region (5′ UTR) different than the naturally occurring 5′ UTR of a human ACE2 mRNA. In a specific embodiment, the 5′ UTR has the nucleotide sequence of SEQ ID NO: 19.

In some embodiments, the mRNA comprises a 3′ untranslated region (3′ UTR) different than the naturally occurring 3′ UTR of a human ACE2 mRNA. In a specific embodiment, the 3′ UTR has the nucleotide sequence of SEQ ID NO: 20 or SEQ ID NO: 21.

Exemplary 5′ and 3′ UTR sequences are shown in Table 5 below:

TABLE 5
Nucleotide sequences of exemplary 5′ untranslated region (5′ UTR)
and 3′ untranslated region (3′ UTR)

| | SEQ ID | |
|---|---|---|
| NAME | NO: | SEQUENCE |
| 5′ UTR | 19 | GGACAGAUCGCCUGGAGACGCCAUCCACGCUGUU |
| sequence | | UUGACCUCCAUAGAAGACACCGGGACCGAUCCAG |
| | | CCUCCGCGGCCGGGAACGGUGCAUUGGAACGCGG |
| | | AUUCCCCGUGCCAAGAGUGACUCACCGUCCUUGAC |
| | | ACG |
| 3′ UTR | 20 | CGGGUGGCAUCCCUGUGACCCCUCCCCAGUGCCUC |
| sequence | | UCCUGGCCCUGGAAGUUGCCACUCCAGUGCCCACC |
| | | AGCCUUGUCCUAAUAAAAUUAAGUUGCAUCAAGC |
| | | U |
| 3′ UTR | 21 | GGGUGGCAUCCCUGUGACCCCUCCCCAGUGCCUCU |
| sequence | | CCUGGCCCUGGAAGUUGCCACUCCAGUGCCCACCA |
| | | GCCUUGUCCUAAUAAAAUUAAGUUGCAUCAAAGC |
| | | U |

Exemplary mRNA Construct

In an exemplary embodiment, an mRNA of the invention comprises the following structural elements, as described in Table 6 below:

TABLE 6
Structural elements of an exemplary mRNA construct

| Structural | | Sequence |
|---|---|---|
| Element | Description | Coordinates |
| 5′ cap | 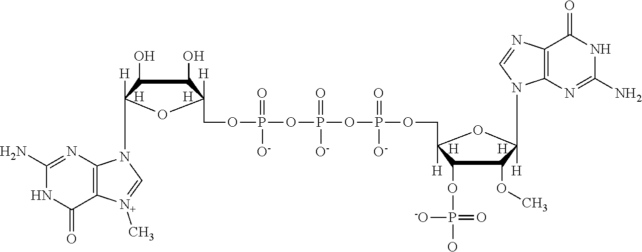 | N/A |
| 5′ UTR | GGACAGAUCGCCUGGAGACGCCAUCCACGCUGU | 1-140 |
| (SEQ ID | UUUGACCUCCAUAGAAGACACCGGGACCGAUCC | |
| NO: 19) | AGCCUCCGCGGCCGGGAACGGUGCAUUGGAACG | |
| | CGGAUUCCCCGUGCCAAGAGUGACUCACCGUCC | |
| | UUGACACG | |
| Optimized | AUGUCUUCCUCUUCUUGGCUGCUGCUGAGCCUG | 141-2363 |
| nucleotide | GUGGCCGUCACAGCCGCUCAGUCCACAAUCGAG | |
| sequence | GAACAGGCUAAAACUUUCCUGGACAAAUUCAA | |
| encoding a | CCACGAAGCAGAGGACCUCUUCUAUCAGUCUAG | |
| polypeptide | CCUGGCCUCCUGGAACUACAACACCAACAUUAC | |
| comprising | UGAGGAGAACGUGCAGAACAUGAACAACGCCG | |
| the | GAGACAAGUGGUCCGCCUUCCUGAAGGAGCAG | |
| extracellular | UCUACGCUGGCACAGAUGUACCCACUGCAGGAG | |
| domain of | AUUCAGAAUCUGACAGUGAAGCUGCAGCUUCA | |
| human | GGCUCUGCAGCAGAACGGGUCUAGUGUGCUGU | |
| angiotensin- | CUGAGGACAAAUCUAAGAGGUUGAACACCAUU | |
| converting | CUGAAUACAAUGUCCACAAUCUAUAGCACCGGC | |
| enzyme 2 | AAGGUGUGCAAUCCUGAUAAUCCUCAGGAGUG | |
| (ACE2) protein | CCUGCUGCUGGAGCCAGGACUGAAUGAAAUUA | |
| (SEQ ID | UGGCUAACUCCCUGGAUUACAACGAAAGACUG | |
| NO: 2) | UGGGCCUGGGAGAGCUGGAGGUCCGAGGUGGG | |
| | AAAGCAGCUGAGACCUCUGUAUGAGGAGUAUG | |
| | UGGUCCUGAAGAAUGAGAUGGCCCGCGCCAACC | |
| | ACUACGAAGACUAUGGAGACUACUGGAGAGGA | |
| | GACUAUGAGGUGAACGGAGUGGAUGGAUAUGA | |
| | CUACAGCAGGGGCCAGCUGAUCGAAGAUGUGG | |
| | AACAUACCUUCGAGGAGAUCAAGCCCCUGUAUG | |
| | AGCAUCUGCACGCAUACGUCAGGGCAAAGCUGA | |
| | UGAACGCCUAUCCCUCCUACAUCAGCCCAAUCG | |
| | GGUGUCUGCCCGCUCACCUCCUGGGGGAUAUGU | |
| | GGGGCAGGUUUUGGACCAAUCUCUACUCUCUG | |
| | ACUGUCCCCUUUGGCCAGAAGCCUAACAUCGAC | |
| | GUGACAGAUGCAAUGGUGGACCAGGCAUGGGA | |
| | CGCCCAGAGGAUCUUCAAGGAAGCAGAGAAAU | |
| | UCUUCGUGUCCGUGGGACUGCCAAAUAUGACCC | |
| | AGGGCUUUUGGGAGAAUAGCAUGCUGACCGAC | |
| | CCAGGGAACGUGCAGAAGGCUGUGUGUCACCCU | |
| | ACAGCCUGGGAUCUGGGGAAGGGCGACUUCAG | |
| | GAUCCUGAUGUGUACAAAGGUUACAAUGGACG | |
| | AUUUCCUGACAGCCCAUCACGAGAUGGGCCAUA | |
| | UCCAGUACGACAUGGCCUAUGCUGCUCAGCCUU | |
| | UUCUGCUGAGGAAUGGAGCAAAUGAGGGGUUC | |
| | CAUGAGGCCGUGGGAGAGAUUAUGUCCCUGUC | |
| | UGCAGCUACCCCUAAACACCUGAAAAGCAUCGG | |
| | CCUGCUGUCACCAGACUUCCAGGAGGACAACGA | |
| | GACAGAGAUCAACUUUCUGCUGAAGCAGGCAC | |
| | UGACAAUCGUGGGGACACUGCCUUUCACCUAUA | |
| | UGCUGGAAAAGUGGAGGUGGAUGGUGUUCAAG | |
| | GGCGAAAUUCCUAAGGACCAGUGGAUGAAGAA | |
| | AUGGUGGGAGAUGAAAAGGGAGAUUGUGGGCG | |
| | UGGUGGAGCCUGUGCCUCACGAUGAGACUUAU | |
| | UGUGACCCCGCCAGCCUGUUUCACGUCUCCAAC | |
| | GACUACUCCUUCAUCAGAUAUUACACAAGAACU | |
| | CUGUACCAGUUCCAGUUCCAGGAGGCCCUGUGU | |
| | CAGGCCGCCAAACACGAGGGGCCACUGCAUAAA | |
| | UGCGAUAUUAGCAACAGCACCGAGGCCGGCCAG | |
| | AAGCUCUUUAAUAUGCUGAGGCUGGGCAAGUC | |
| | CGAGCCUUGGACUCUAGCCCUGGAGAACGUGGU | |
| | GGGAGCAAAGAACAUGAAUGUGAGACCACUGC | |
| | UGAACUACUUCGAGCCCCUGUUUACCUGGCUGA | |
| | AAGACCAGAACAAAAACUCUUUUGUGGGCUGG | |
| | AGCACCGAUUGGUCUCCAUACGCAGACCAGAGC | |
| | AUCAAAGUCCGCAUCUCCCUGAAAUCAGCCCUG | |
| | GGGGAUAAGGCCUAUGAGUGGAAUGACAAUGA | |
| | AAUGUACCUCUUUCGGUCCAGCGUGGCAUAUGC | |
| | UAUGCGGCAGUACUUCCUGAAGGUCAAGAACC | |
| | AGAUGAUCCUGUUUGGCGAGGAAGACGUGAGG | |
| | GUUGCCAAUCUGAAGCCCCGCAUUUCUUUUAAC | |
| | UUUUUCGUCACUGCCCCCAAGAACGUGAGCGAC | |
| | AUCAUUCCAAGGACCGAAGUGGAAAAGGCUAU | |
| | CAGAAUGUCUCGGUCCAGGAUCAACGACGCCUU | |
| | CAGGUUGAACGACAACAGCCUGGAGUUUCUGG | |
| | GGAUUCAGCCUACACUGGGGCCUCCCAAUCAGC | |
| | CACCCGUGAGCUGA | |
| 3′ UTR | CGGGUGGCAUCCCUGUGACCCCUCCCCAGUGCC | 2364-2468 |
| (SEQ ID | UCUCCUGGCCCUGGAAGUUGCCACUCCAGUGCC | |
| NO: 20) | CACCAGCCUUGUCCUAAUAAAAUUAAGUUGCA | |
| | UCAAGCU | |
| Poly(A) tail | (A)ₓ, x = 100-500* | N/A |

*expected range
N/A = not applicable

SEQ TD NO: 2 is shown merely for illustrative purposes. It may be replaced with any of the optimized nucleotide sequences encoding a polypeptide comprising the extracellular domain of human ACE2 protein disclosed herein (e.g., those listed in Table 3). For example, in some embodiments, SEQ ID NO: 2 in Table 6 may be replaced with SEQ TD NO: 4. In other embodiments, SEQ ID NO: 2 in Table 6 may be replaced with SEQ ID NO: 5.

### Nucleotides

In some embodiments, the mRNA comprises or consists of naturally-occurring nucleosides (or unmodified nucleosides; i.e., adenosine, guanosine, cytidine, and uridine). In some embodiments, the mRNA comprises one or more modified nucleosides, such as nucleoside analogs (e.g. adenosine analog, guanosine analog, cytidine analog, or uridine analog). The presence of one or more nucleoside analogs may render an mRNA more stable and/or less immunogenic than a control mRNA with the same sequence but containing only naturally-occurring nucleosides. In a particular embodiment of the invention, mRNAs comprising an optimized nucleotide sequence encoding a polypeptide comprising the extracellular domain of human ACE2 protein are synthesized with naturally-occurring nucleosides. Without wishing to be bound by any particular theory, the inventors believe that the use of mRNAs prepared with naturally-occurring nucleosides is advantageous for providing a composition or therapeutic composition of the invention.

In some embodiments, the mRNA comprises both unmodified and modified nucleosides. In some embodiments, the one or more modified nucleosides is a nucleoside analog. In some embodiments, the one or more modified nucleosides comprises at least one modification selected from a modified sugar, and a modified nucleobase. In some embodiments, the mRNA comprises one or more modified internucleoside linkages.

In some embodiments, the one or more modified nucleosides is a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N-1-methyl-pseudouridine), 2-thiouridine, and 2-thiocytidine. See, e.g., U.S. Pat. No. 8,278,036 or WO 2011/012316 for a discussion of 5-methyl-cytidine, pseudouridine, and 2-thio-uridine and their incorporation into mRNA. In some embodiments, the mRNA may be RNA wherein 25% of U residues are 2-thio-uridine and 25% of C residues are 5-methylcytidine. Teachings for the use of such modified RNA are disclosed in US Patent Publication US 2012/0195936 and international publication WO 2011/012316, both of which are hereby incorporated by reference in their entirety.

### In Vitro Transcription

mRNAs of the invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template or DNA vector containing a promoter, a pool of ribonucleotide triphosphates, a buffer system that may include DTT and magnesium ions, and an appropriate RNA polymerase (e.g., T3, T7, or SP6 RNA polymerase), DNase I, pyrophosphatase, and/or RNase inhibitor. The exact conditions will vary according to the specific application.

For the preparation of mRNA by IVT, a DNA template or DNA vector may be transcribed in vitro. A suitable DNA template or DNA vector typically has a promoter, for example a T3, T7 or SP6 promoter, for in vitro transcription, followed by desired nucleotide sequence for desired mRNA and a termination signal (terminator).

In one aspect, the invention provides a DNA vector encoding an mRNA comprising an optimized nucleotide sequence encoding a polypeptide comprising the extracellular domain of human angiotensin-converting enzyme 2 (ACE2) protein, or a portion thereof, which binds to a viral surface protein. In some embodiments, the DNA vector further comprises a promoter and/or a terminator. In one embodiment, the promoter is a SP6 RNA polymerase promoter. In another embodiment, the promoter is a T7 RNA polymerase promoter.

### Post-Synthesis Purification

Various methods may be used to purify mRNA after synthesis. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention. It is advantageous to purify the mRNA of the invention which may be included in pharmaceutical compositions in some embodiments of the invention, as the purity requirements for mRNA products are more stringent for therapeutic applications.

In some embodiments, the mRNA is purified before capping and tailing. In some embodiments, the mRNA is purified after capping and tailing. In some embodiments, the mRNA is purified both before and after capping and tailing. In some embodiments, the mRNA is purified either before or after or both before and after capping and tailing, by centrifugation. In some embodiments, the mRNA is purified either before or after or both before and after capping and tailing, by filtration. In some embodiments, the mRNA is purified either before or after or both before and after capping and tailing, by Tangential Flow Filtration (TFF).

### Lipid Nanoparticles (LNPs)

The invention also provides a lipid nanoparticle (LNP) encapsulating an mRNA of the invention. Typically, a lipid nanoparticle suitable for use with the present invention comprises one or more cationic lipids, one or more non-cationic lipids (e.g., DOPE and/or cholesterol), and one or more PEG-modified lipids (e.g., DMG-PEG2K).

A typical lipid nanoparticle for use with the invention is composed of four lipid components: a cationic lipid (e.g., a sterol-based cationic lipid), a non-cationic lipid (e.g., DOPE or DEPE), a cholesterol-based lipid (e.g., cholesterol) and a PEG-modified lipid (e.g., DMG-PEG2K). In a specific embodiment, the non-cationic lipid is DOPE. The molar ratio of cationic lipid to non-cationic lipid to cholesterol to PEG-modified lipid typically is between about 30-60:25-35:20-30:1-15, respectively. An exemplary LNP in accordance with the invention may be composed of a cationic lipid selected from cKK-E12, cKK-E10, OF-Deg-Lin and OF-02; a non-cationic lipid selected from DOPE and DEPE; a cholesterol-based lipid such as cholesterol; and a PEG-modified lipid such as DMG-PEG2K.

In some embodiments, a lipid nanoparticle comprises no more than three distinct lipid components. An exemplary lipid nanoparticle is composed of three lipid components: a cationic lipid (e.g., a sterol-based cationic lipid), a non-cationic lipid (e.g., DOPE or DEPE) and a PEG-modified lipid (e.g., DMG-PEG2K). In a specific embodiment, the three distinct lipid components are HGT4002, DOPE and DMG-PEG2K. In an exemplary embodiment, HGT4002, DOPE and DMG-PEG2K are present in a molar ratio of approximately 60:35:5, respectively. Such LNPs may be particularly suitable for aerosol delivery of the mRNAs of the invention.

The lipid nanoparticles for use in the invention can be prepared by various techniques which are presently known in the art. Such methods are described, e.g., in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019, all of which are incorporated herein by reference.

### Lipid Nanoparticle Formulations

In some embodiments, the majority of LNPs in a composition of the invention, i.e., greater than about 50%, 55%, 60%, 65%, 70%, 75%, 80%, 85%, 90%, 95%, 96%, 97%, 98%, or 99% of the LNPs, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, the LNPs in a composition of the invention have a size of about 150 nm or less (e.g., about 145 nm or less, about 140 nm or less, about 135 nm or less, about 130 nm or less, about 125 nm or less, about 120 nm or less, about 115 nm or less, about 110 nm or less, about 105 nm or less, about 100 nm or less, about 95 nm or less, about 90 nm or less, about 85 nm or less, or about 80 nm or less).

In some embodiments, greater than about 70%, 75%, 80%, 85%, 90%, 95%, 96%, 97%, 98%, 99% of the LNPs in a composition provided by the present invention have a size ranging from about 40-90 nm (e.g., about 45-85 nm, about 50-80 nm, about 55-75 nm, about 60-70 nm). In some embodiments, the LNPs have a size ranging from about 40-90 nm (e.g., about 45-85 nm, about 50-80 nm, about 55-75 nm, about 60-70 nm). Compositions with LNPs having an average size of about 50-70 nm (e.g., 55-65 nm) may be particularly suitable for pulmonary delivery via nebulization.

In some embodiments, the dispersity, or measure of heterogeneity in size of molecules (PDI), of lipid nanoparticles in a pharmaceutical composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

LNPs may be prepared by combining mRNA either with a mixture of cationic, non-cationic and PEG-modified lipids or with pre-formed, empty LNPs. For example, LNPs may be prepared by rapidly injection the mRNA in an aqeuous solution into a solvent comprising the lipid mixture. The mRNA and cationic lipid may be present at an equimolar ratio to achieve encapsulation. More typically, LNPs are prepared with a molar excess of the cationic lipid per mole mRNA. Accordingly, in some embodiments, an LNP has an N/P ratio between 1 and 6. In some embodiments, an LNP has an N/P ratio between 3-5. In some embodiments, an LNP has an N/P ratio of about 4.

In some embodiments, an LNP has an encapsulation efficiency of greater than about 80%. In some embodiments, an LNP has an encapsulation efficiency of greater than about 85%. In some embodiments, an LNP has an encapsulation efficiency of greater than about 90%. In some embodiments, an LNP has an encapsulation efficiency of greater than about 92%. In some embodiments, an LNP has an encapsulation efficiency of greater than about 95%. In some embodiments, an LNP has an encapsulation efficiency of greater than about 98%. In some embodiments, an LNP has an encapsulation efficiency of greater than about 99%. Typically, LNPs for use with the invention have an encapsulation efficiency of at least 90%-95%.

### Cationic Lipids

Various cationic lipids which are suitable for use in LNPs are known in the art. These include, for example, DOTAP (1,2-dioleyl-3-trimethylammonium propane), DODAP (1,2-dioleyl-3-dimethylammonium propane), DOTMA (N-[1-(2,3-dioleyloxy)propyl]-N,N,N-trimethylammonium chloride), DLinKC2DMA, DLin-KC2-DM, and C12-200. Exemplary cationic lipids suitable for use in the LNPs, compositions, pharmaceutical compositions and methods of the invention are described herein and include, for instance, the cationic lipids as described in International Patent Publication WO 2010/144740, which is incorporated herein by reference. In some embodiments, the LNPs, compositions, pharmaceutical compositions and methods of the present invention include a cationic lipid, (6Z,9Z,28Z,31Z)-heptatriaconta-6,9,28,31-tetraen-19-yl 4-(dimethylamino) butanoate, having a compound structure of:

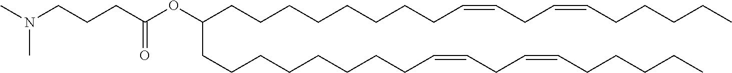 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the LNPs, compositions, pharmaceutical compositions and methods of the present invention include ionizable cationic lipids as described in International Patent Publication WO 2013/149140, which is incorporated herein by reference. In some embodiments, the LNPs, compositions, pharmaceutical compositions and methods of the present invention include a cationic lipid of one of the following formulas:

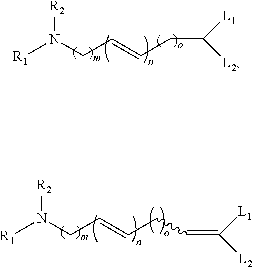

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In some embodiments, the LNPs, compositions, pharmaceutical compositions and methods of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

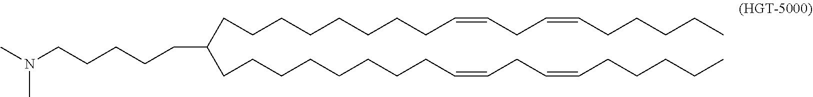 [SMILES: CCCCC/C=C\C/C=C\CCCCCCCCC(CCCCCCCC/C=C\C/C=C\CCCCC)CCCCCN(C)C]

and pharmaceutically acceptable salts thereof. In some embodiments, the LNPs, compositions, pharmaceutical compositions and methods of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

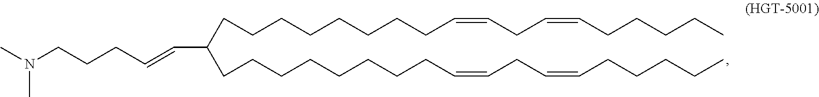 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In some embodiments, the LNPs, compositions, pharmaceutical compositions and methods of the present invention include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

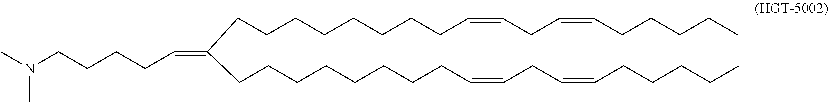 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the LNPs, compositions, pharmaceutical compositions and methods of the invention include cationic lipids described as aminoalcohol lipidoids in International Patent Publication WO 2010/053572, which is incorporated herein by reference. In some embodiments, the LNPs, compositions, pharmaceutical compositions and methods of the present invention include a cationic lipid having a compound structure of:

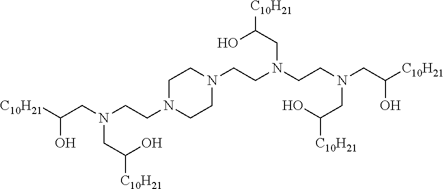

and pharmaceutically acceptable salts thereof.

Other suitable cationic lipids for use in the LNPs, compositions, pharmaceutical compositions and methods of the invention include the cationic lipids as described in International Patent Publication WO 2016/118725, which is incorporated herein by reference. In some embodiments, the LNPs, compositions, pharmaceutical compositions and methods of the present invention include a cationic lipid having a compound structure of:

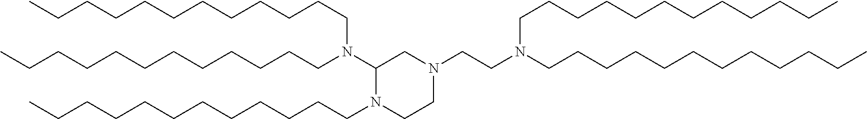 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the LNPs, compositions, pharmaceutical compositions and methods of the invention include the cationic lipids as described in International Patent Publication WO 2016/118724, which is incorporated herein by reference. In some embodiments, the LNPs, compositions, pharmaceutical compositions and methods of the present invention include a cationic lipid having a compound structure of:

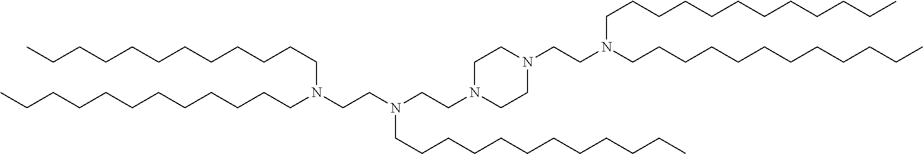 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the LNPs, compositions, pharmaceutical compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the LNPs, compositions, pharmaceutical compositions and methods of the invention include the cationic lipids as described in International Patent Publications WO 2013/063468 and WO 2016/205691, each of which are incorporated herein by reference. In some embodiments, the LNPs, compositions, pharmaceutical compositions and methods of the present invention include a cationic lipid of the following formula:

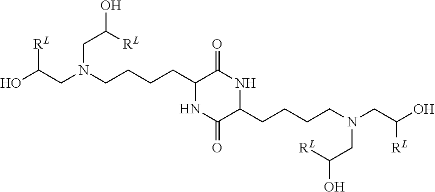

or pharmaceutically acceptable salts thereof, wherein each instance of R^{L} is independently optionally substituted C₆-C₄₀ alkenyl. In some embodiments, the LNPs, compositions, pharmaceutical compositions and methods of the present invention include a cationic lipid having a compound structure of:

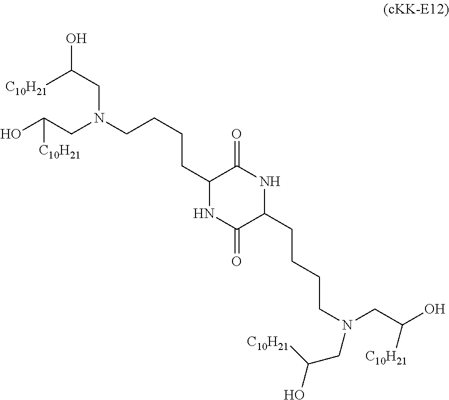

and pharmaceutically acceptable salts thereof. In some embodiments, the LNPs, compositions, pharmaceutical compositions and methods of the present invention include a cationic lipid having a compound structure of:

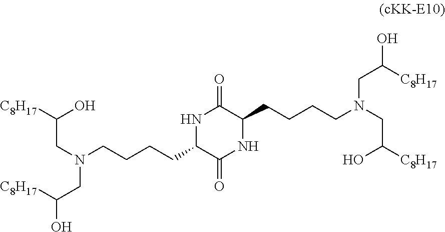

and pharmaceutically acceptable salts thereof.

In some embodiments, the LNPs, compositions, pharmaceutical compositions and methods of the present invention include a cationic lipid having a compound structure of:

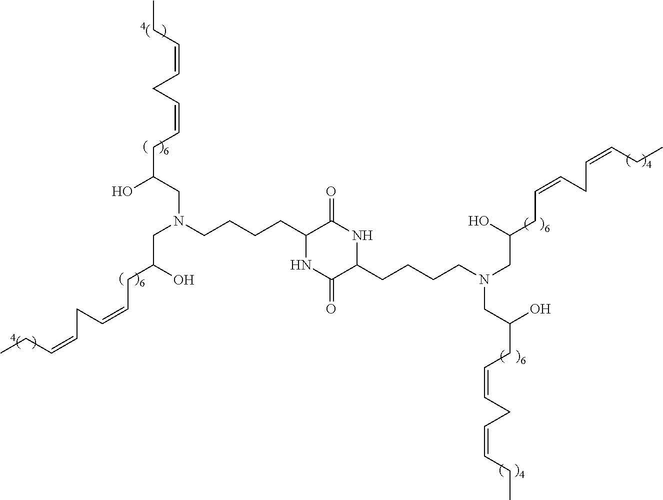

and pharmaceutically acceptable salts thereof. In some embodiments, the LNPs, compositions, pharmaceutical compositions and methods of the present invention include a cationic lipid having a compound structure of:

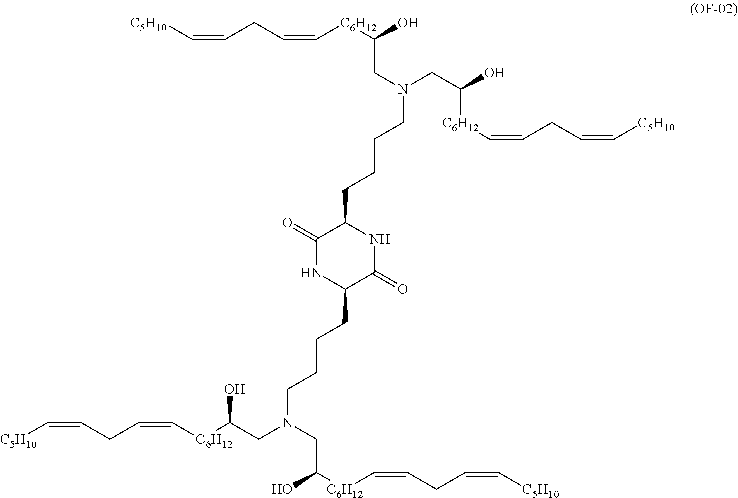

and pharmaceutically acceptable salts thereof. In some embodiments, the LNPs, compositions, pharmaceutical compositions and methods of the present invention include a cationic lipid having a compound structure of:

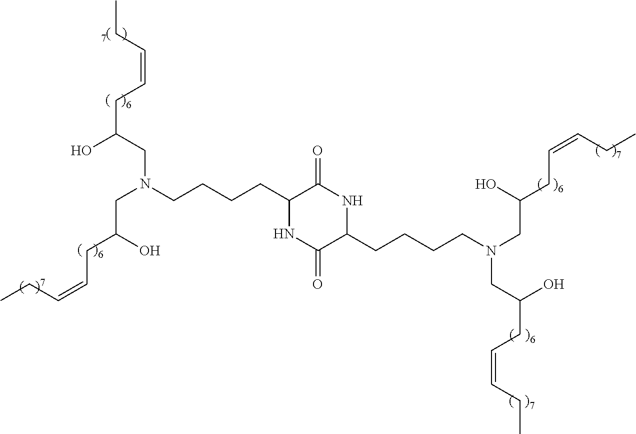

and pharmaceutically acceptable salts thereof. In some embodiments, t e LNPs, compositions, pharmaceutical compositions and methods of the present invention include a cationic lipid having a compound structure of:

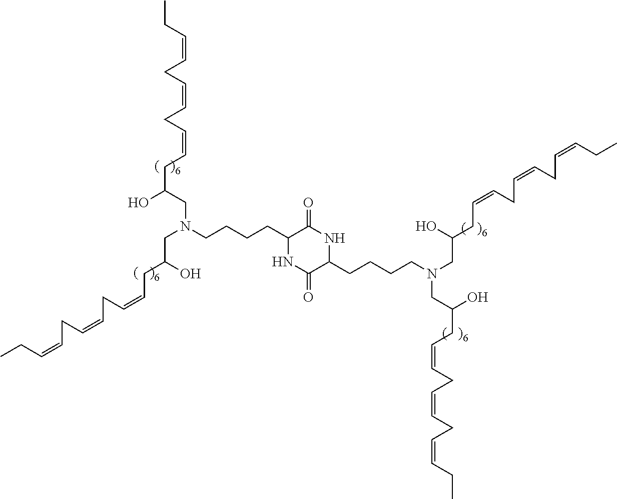

and pharmaceutically acceptable salts thereof.

Other suitable cationic lipids for use in the LNPs, compositions, pharmaceutical compositions and methods of the invention include the cationic lipids as described in International Patent Publication WO 2015/184256, which is incorporated herein by reference. In some embodiments, the LNPs, compositions, pharmaceutical compositions and methods of the present invention include a cationic lipid of the following formula:

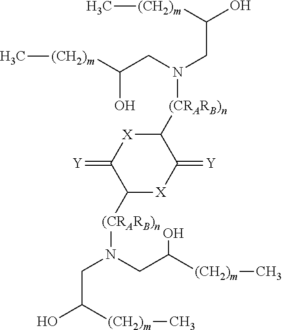

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In some embodiments, the LNPs, compositions, pharmaceutical compositions and methods of the present invention include a cationic lipid, “Target 23”, having a compound structure of:

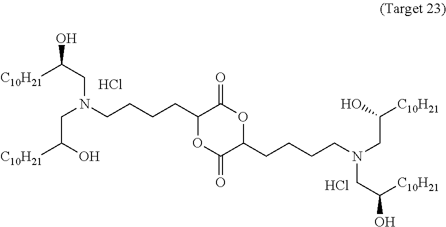

and pharmaceutically acceptable salts thereof.

Other suitable cationic lipids for use in the LNPs, compositions, pharmaceutical compositions and methods of the invention include the cationic lipids as described in International Patent Publication WO 2016/004202, which is incorporated herein by reference. In some embodiments, the LNPs, compositions, pharmaceutical compositions and methods of the present invention include a cationic lipid having the compound structure:

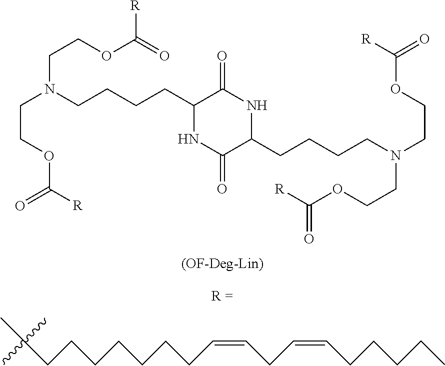

or a pharmaceutically acceptable salt thereof. In some embodiments, the LNPs, compositions, pharmaceutical compositions and methods of the present invention include a cationic lipid having the compound structure:

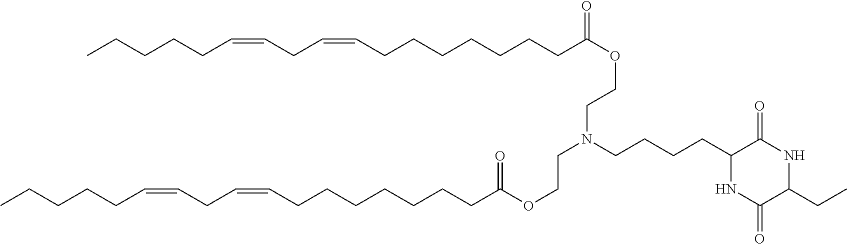 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the LNPs, compositions, pharmaceutical compositions and methods of the present invention include a cationic lipid having the compound structure:

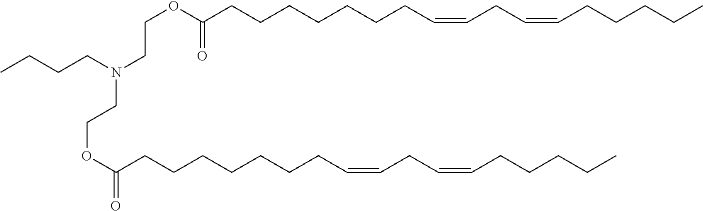 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the LNPs, compositions, pharmaceutical compositions and methods of the present invention include cationic lipids as described in International Patent Publication WO 2020/097384, which is incorporated herein by reference. In some embodiments, the LNPs, compositions, pharmaceutical compositions and methods of the present invention include a cationic lipid of the following formula:

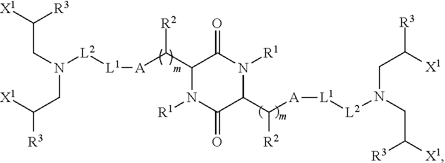

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the LNPs, compositions, pharmaceutical compositions and methods of the present invention include a cationic lipid of the following formula:

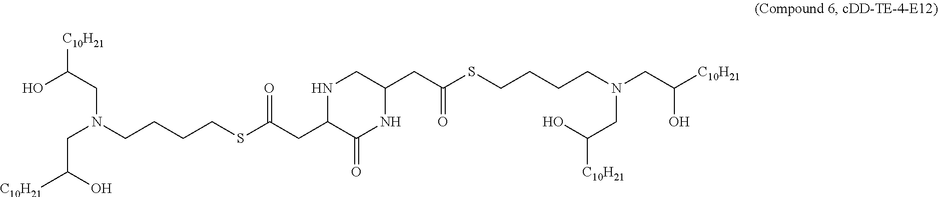

or a pharmaceutically acceptable salt thereof. In some embodiments, the LNPs, compositions, pharmaceutical compositions and methods of the present invention include a cationic lipid of the following formula:

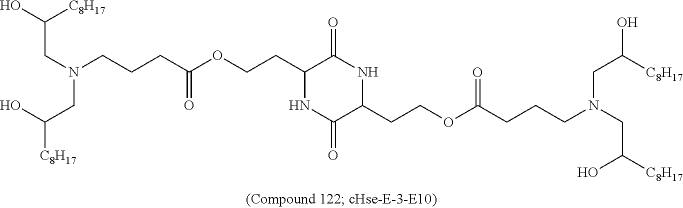

or a pharmaceutically acceptable salt thereof. In some embodiments, the LNPs, compositions, pharmaceutical compositions and methods of the present invention include a cationic lipid of the following formula:

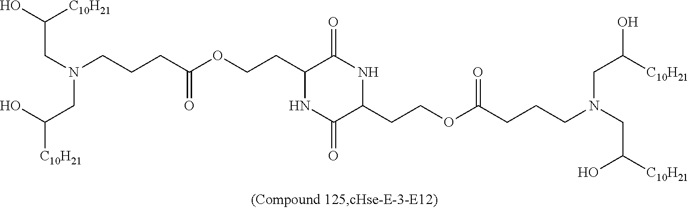

or a pharmaceutically acceptable salt thereof.

Other suitable cationic lipids for use in the LNPs, compositions, pharmaceutical compositions and methods of the invention include the cationic lipids as described in International Patent Publication WO 2021/202694, which is incorporated herein by reference. In some embodiments, the LNPs, compositions, pharmaceutical compositions and methods of the present invention include a cationic lipid of the following formula:

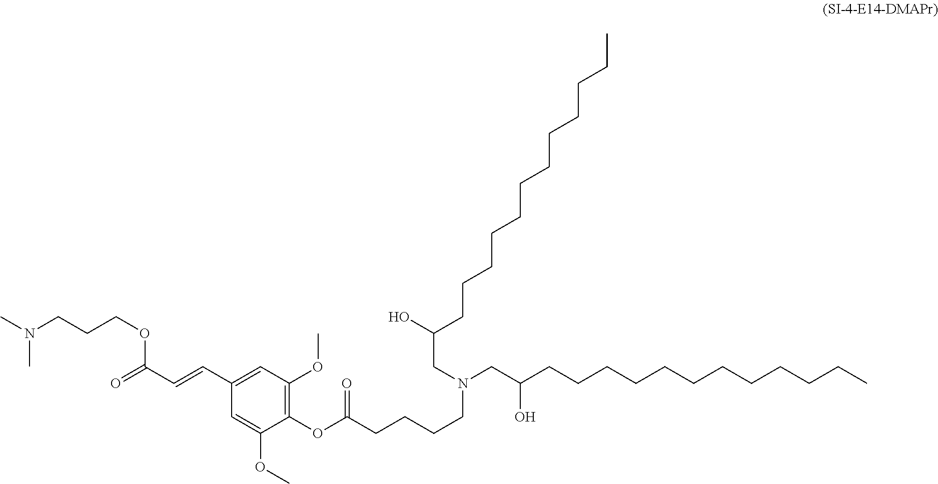 [SMILES: CCCCCCCCCCCCC(O)CN(CCCCC(=O)Oc1c(OC)cc(/C=C/C(=O)OCCCN(C)C)cc1OC)CC(O)CCCCCCCCCCCC]

or a pharmaceutically acceptable salt thereof. Other suitable cationic lipids for use in the pharmaceutical compositions and methods of the present invention include the cationic lipids as described in J. McClellan, M. C. King, Cell 2010, 141, 210-217 and in Whitehead et al., Nature Communications (2014) 5:4277, which is incorporated herein by reference. In some embodiments, the cationic lipids of the LNPs, compositions, pharmaceutical compositions and methods of the present invention include a cationic lipid having a compound structure of:

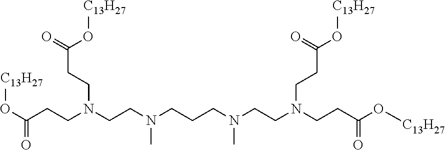

and pharmaceutically acceptable salts thereof. In some embodiments, the LNPs, compositions, pharmaceutical compositions and methods of the present invention include a cationic lipid having a compound structure of:

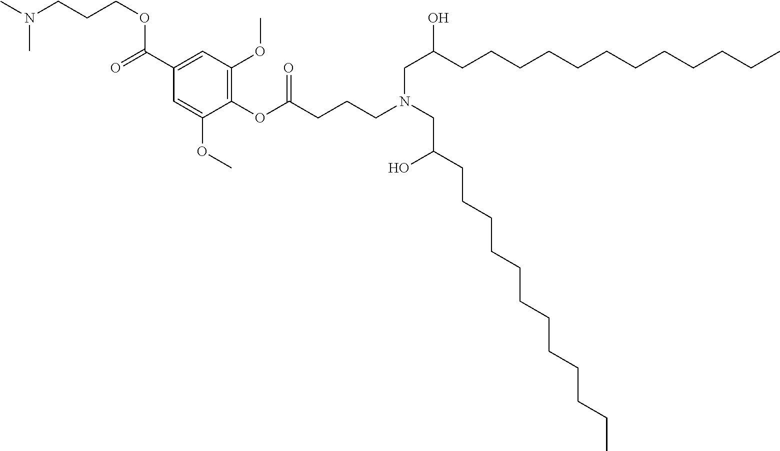 [SMILES: CCCCCCCCCCCCC(O)CN(CCCC(=O)Oc1c(OC)cc(C(=O)OCCCN(C)C)cc1OC)CC(O)CCCCCCCCCCCC]

(SY-3-E14-DMAPr) and pharmaceutically acceptable salts thereof. In some embodiments, the LNPs, compositions, pharmaceutical compositions and methods of the present invention include a cationic lipid of the following formula.

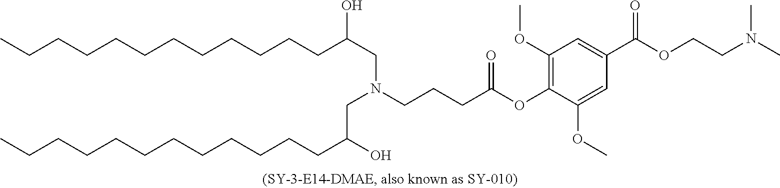 [SMILES: CCCCCCCCCCCCC(O)CN(CCCC(=O)Oc1c(OC)cc(C(=O)OCCN(C)C)cc1OC)CC(O)CCCCCCCCCCCC]

or a pharmaceutically acceptable salt thereof. In some embodiments, the LNPs, compositions, pharmaceutical compositions and methods of the present invention include a cationic lipid of the following formula:

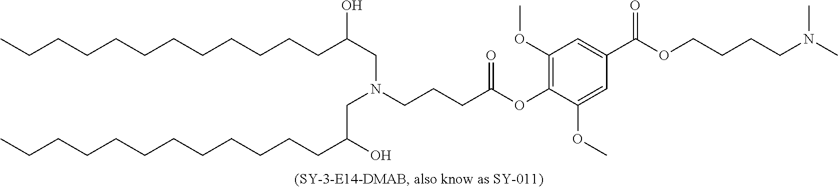 [SMILES: CCCCCCCCCCCCC(O)CN(CCCC(=O)Oc1c(OC)cc(C(=O)OCCCCN(C)C)cc1OC)CC(O)CCCCCCCCCCCC]

or a pharmaceutically acceptable salt thereof.

Other suitable cationic lipids for use in the LNPs, compositions, pharmaceutical compositions and methods of the invention include the cationic lipids as described in International Patent Publication WO 2015/199952, which is incorporated herein by reference. In some embodiments, the LNPs, compositions, pharmaceutical compositions and methods of the present invention include a cationic lipid having the compound structure:

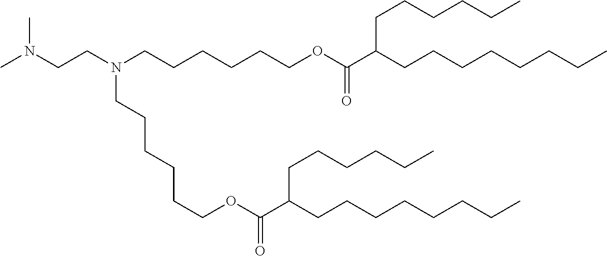 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

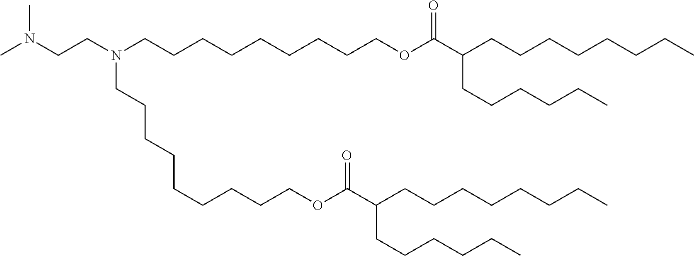 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

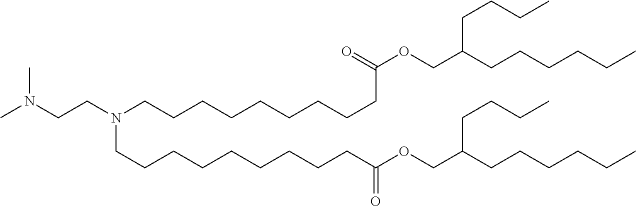 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the LNPs, compositions, pharmaceutical compositions and methods of the present invention include a cationic lipid having the compound structure:

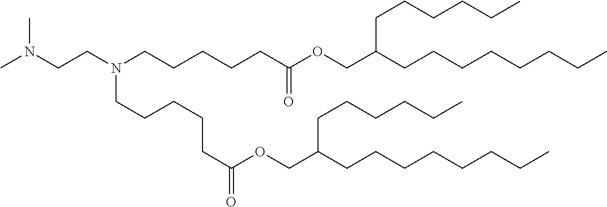 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

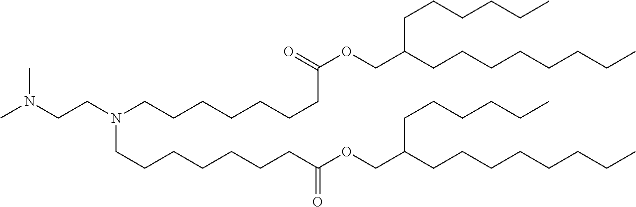 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

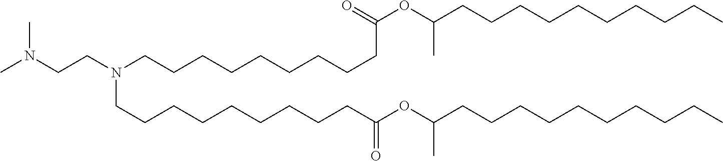 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

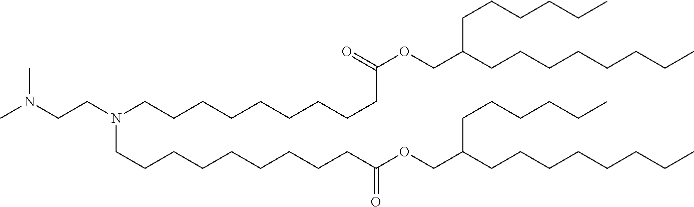 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the LNPs, compositions, pharmaceutical compositions and methods of the present invention include a cationic lipid having the compound structure:

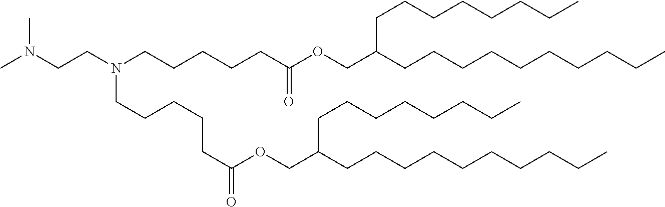 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

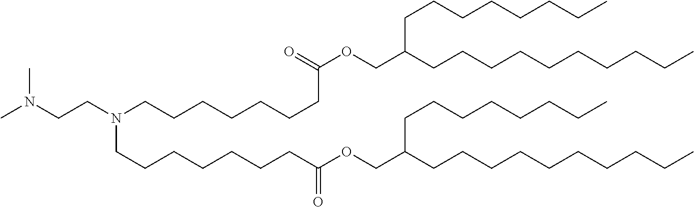 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the LNPs, compositions, pharmaceutical compositions and methods of the present invention include a cationic lipid having the compound structure:

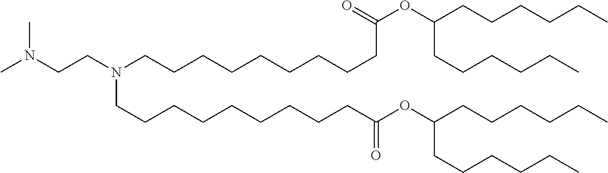 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the LNPs, compositions, pharmaceutical compositions and methods of the present invention include a cationic lipid having the compound structure:

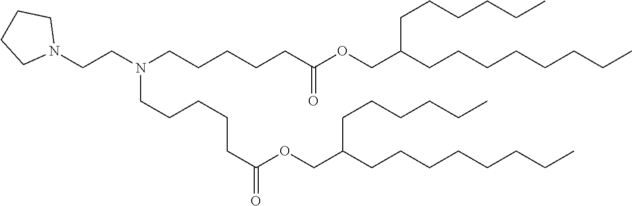 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the LNPs, compositions, pharmaceutical compositions and methods of the present invention include a cationic lipid having the compound structure:

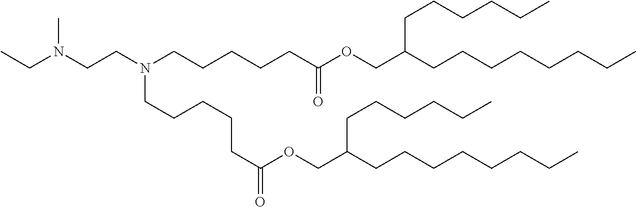 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the LNPs, compositions, pharmaceutical compositions and methods of the present invention include a cationic lipid having the compound structure:

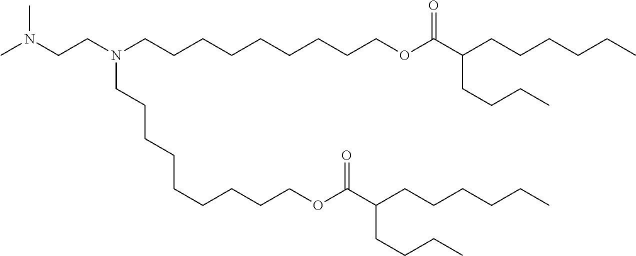 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/004143, which is incorporated herein by reference. In some embodiments, the LNPs, compositions, pharmaceutical compositions and methods of the present invention include a cationic lipid having the compound structure:

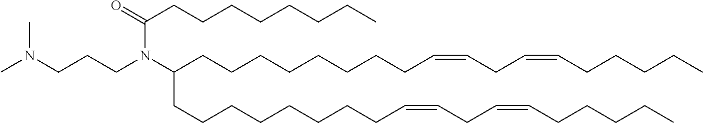 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the LNPs, compositions, pharmaceutical compositions and methods of the present invention include a cationic lipid having the compound structure:

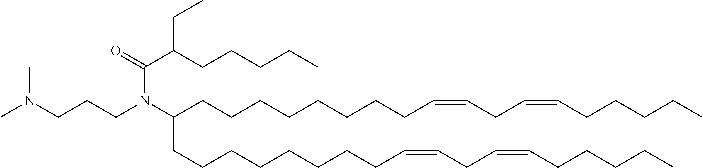 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

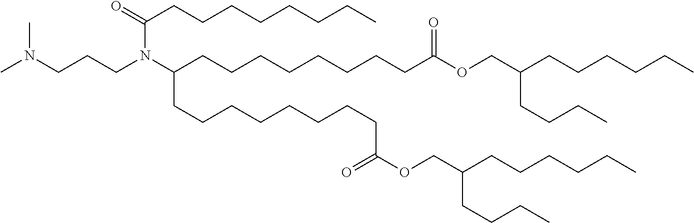 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

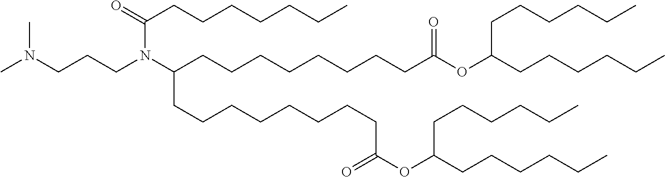 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

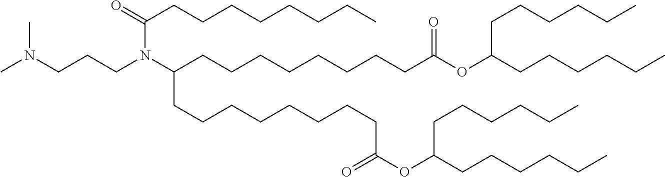 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the LNPs, compositions, pharmaceutical compositions and methods of the present invention include a cationic lipid having the compound structure:

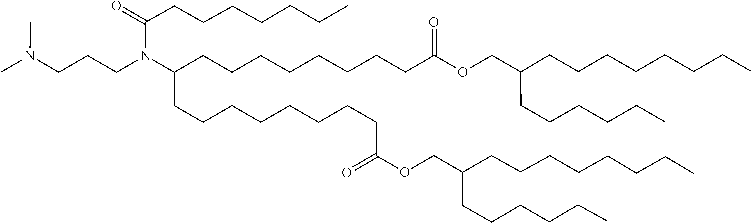 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

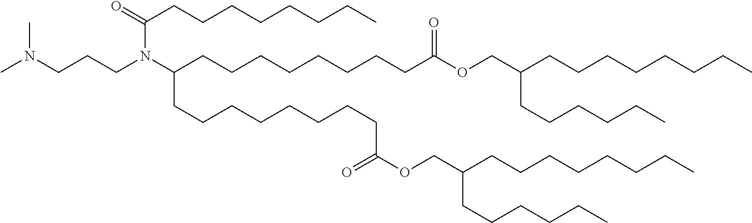 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

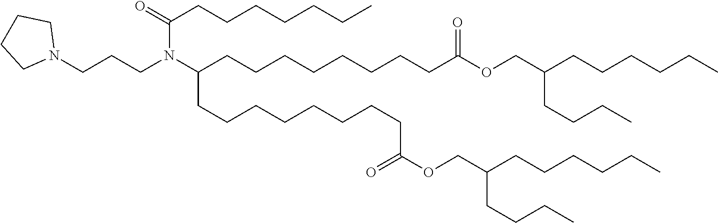 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

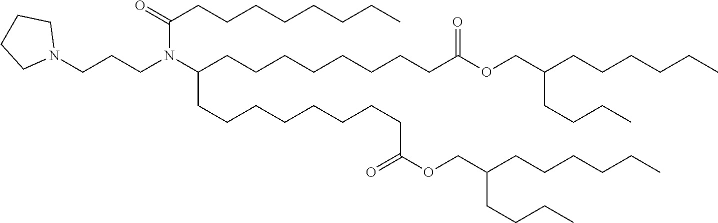 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the LNPs, compositions, pharmaceutical compositions and methods of the present invention include a cationic lipid having the compound structure:

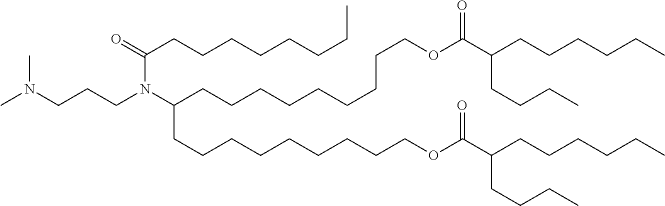 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the LNPs, compositions, pharmaceutical compositions and methods of the present invention include a cationic lipid having the compound structure:

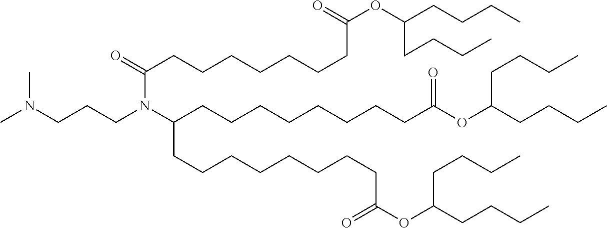 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the LNPs, compositions, pharmaceutical compositions and methods of the present invention include a cationic lipid having the compound structure:

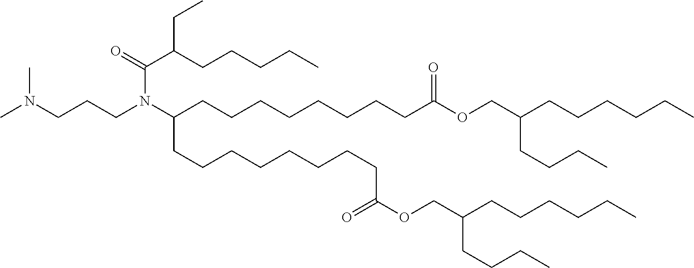 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the LNPs, compositions, pharmaceutical compositions and methods of the present invention include a cationic lipid having the compound structure:

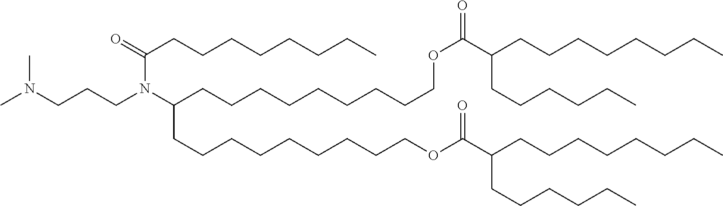 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the LNPs, compositions, pharmaceutical compositions and methods of the present invention include a cationic lipid having the compound structure:

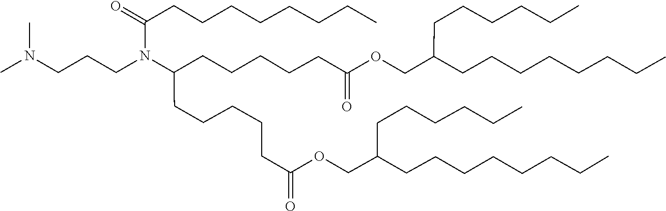 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the LNPs, compositions, pharmaceutical compositions and methods of the present invention include a cationic lipid having the compound structure:

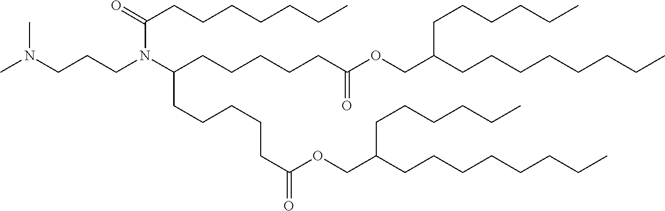 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the LNPs, compositions, pharmaceutical compositions and methods of the present invention include a cationic lipid having the compound structure:

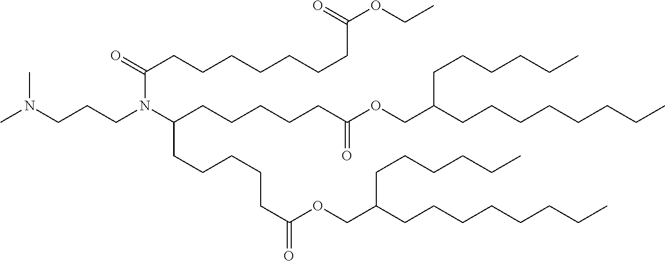 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the LNPs, compositions, pharmaceutical compositions and methods of the present invention include a cationic lipid having the compound structure:

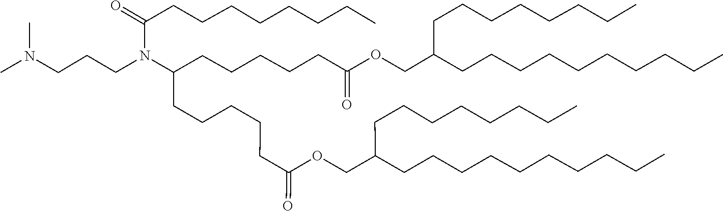 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/075531, which is incorporated herein by reference. In some embodiments, the LNPs, compositions, pharmaceutical compositions and methods of the present invention include a cationic lipid of the following formula:

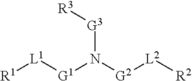

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the LNPs, compositions, pharmaceutical compositions and methods of the invention include the cationic lipids as described in International Patent Publication WO 2017/117528, which is incorporated herein by reference. In some embodiments, the LNPs, compositions, pharmaceutical compositions and methods of the present invention include a cationic lipid having the compound structure:

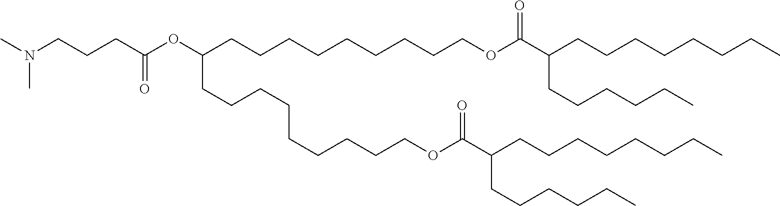 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

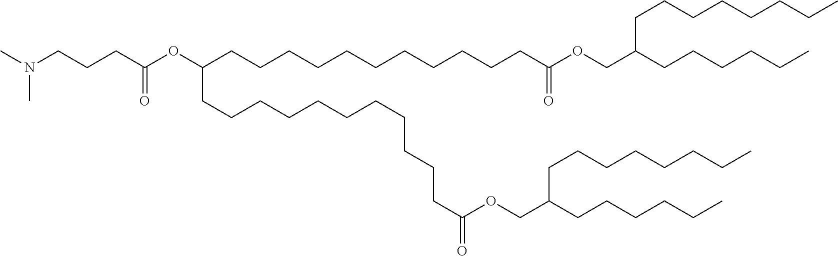 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the LNPs, compositions, pharmaceutical compositions and methods of the present invention include a cationic lipid having the compound structure:

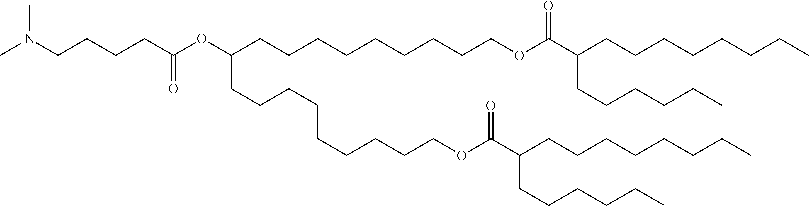 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the LNPs, compositions, pharmaceutical compositions and methods of the invention include the cationic lipids as described in International Patent Publication WO 2017/049245, which is incorporated herein by reference. In some embodiments, the cationic lipids of the LNPs, compositions, pharmaceutical compositions and methods of the present invention include a compound of one of the following formulas:

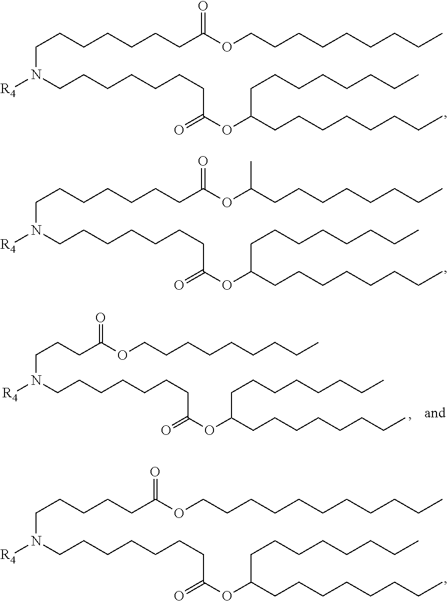

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In some embodiments, the LNPs, compositions, pharmaceutical compositions and methods of the present invention include a cationic lipid having a compound structure of:

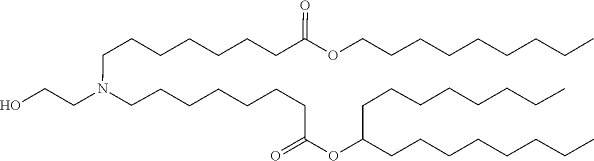 [SMILES: CCCCCCCCCOC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

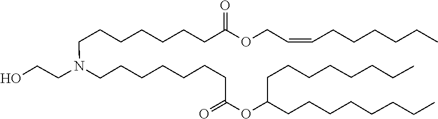 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

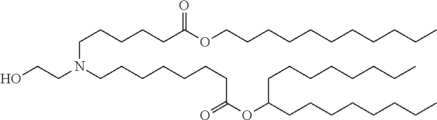 [SMILES: CCCCCCCCCCCOC(=O)CCCCCN(CCO)CCCCCCCC(=O)OC(CCCCCCCC)CCCCCCCC]

and pharmaceutically acceptable salts thereof. In some embodiments, the LNPs, compositions, pharmaceutical compositions and methods of the present invention include a cationic lipid having a compound structure of:

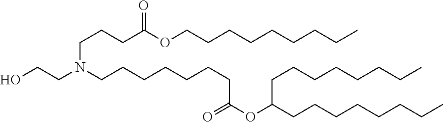 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the LNPs, compositions, pharmaceutical compositions and methods of the invention include the cationic lipids as described in International Patent Publication WO 2017/173054 and WO 2015/095340, each of which is incorporated herein by reference. In some embodiments, the LNPs, compositions, pharmaceutical compositions and methods of the present invention include a cationic lipid having a compound structure of:

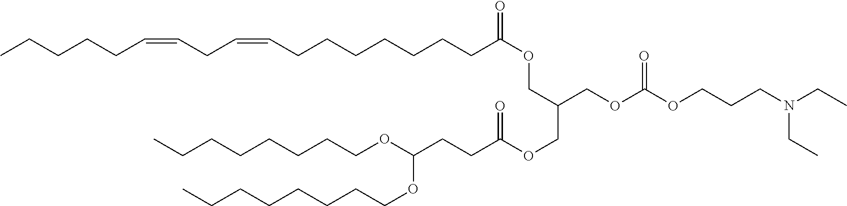 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In some embodiments, the LNPs, compositions, pharmaceutical compositions and methods of the present invention include a cationic lipid having a compound structure of:

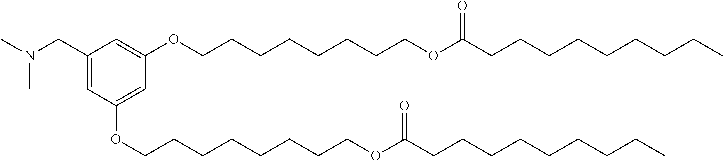 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In some embodiments, the LNPs, compositions, pharmaceutical compositions and methods of the present invention include a cationic lipid having a compound structure of:

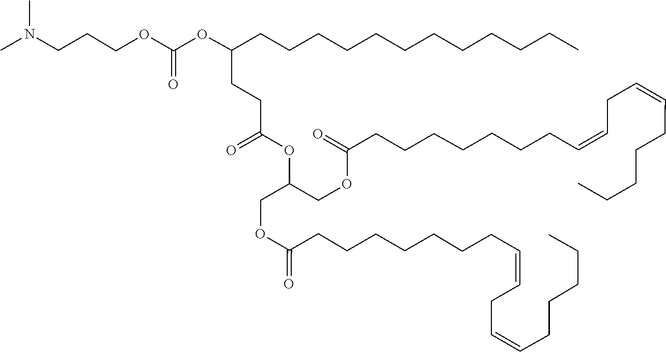 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In some embodiments, the LNPs, compositions, pharmaceutical compositions and methods of the present invention include a cationic lipid having a compound structure of

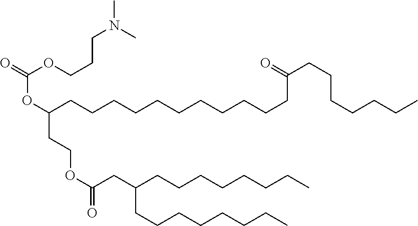 [SMILES: CCCCCCCCC(CCCCCCCC)CC(=O)OCCC(CCCCCCCCCCCC(=O)CCCCCCC)OC(=O)OCCCN(C)C]

and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 63/082,090, filed on Sep. 23, 2020, and International Patent Application PCT/US2021/051403 which are incorporated herein by reference. In some embodiments, the pharmaceutical compositions and methods of the present invention include a cationic lipid having a compound structure of

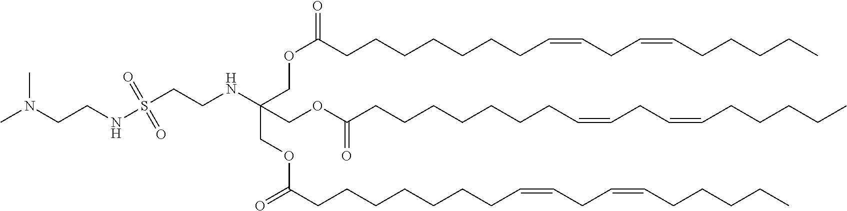 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

(GL-TES-SA-DME-E18-2) and pharmaceutically acceptable salts thereof.

In some embodiments, the LNPs, compositions, pharmaceutical compositions and methods of the present invention include a cationic lipid having a compound structure of:

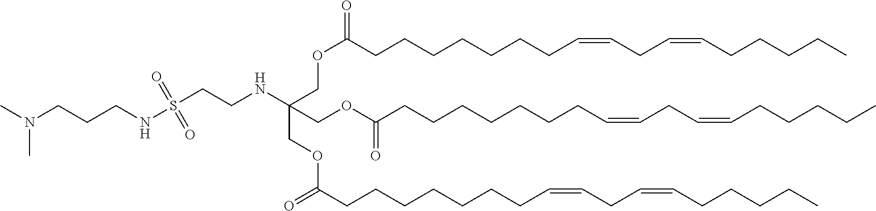 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

(GL-TES-SA-DMP-E18-2) and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 63/082,101, filed on Sep. 23, 2020 and International Patent Application PCT/US2021/51763, which are incorporated herein by reference. In some embodiments, the LNPs, compositions, pharmaceutical compositions and methods of the present invention include a cationic lipid having a compound structure of:

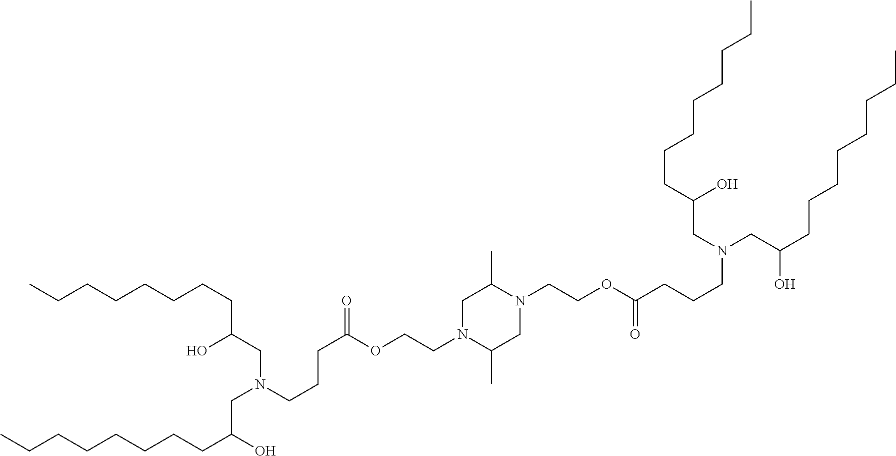 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

(HEP-E3-E10) and pharmaceutically acceptable salts thereof.

In some embodiments, the LNPs, compositions, pharmaceutical compositions and methods of the present invention include a cationic lipid having a compound structure of:

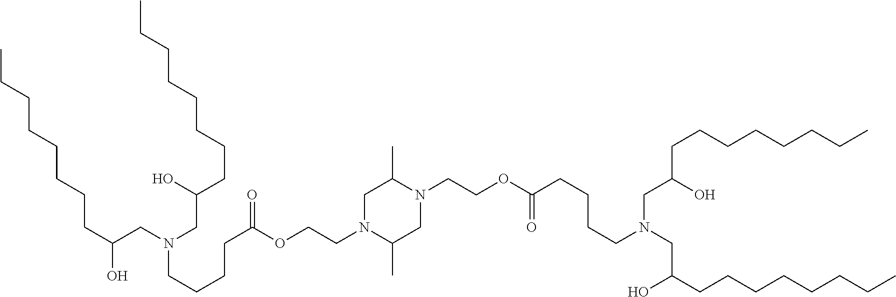 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

(HEP-E4-E10) and pharmaceutically acceptable salts thereof.

Other suitable cationic lipids for use in the LNPs, compositions, pharmaceutical compositions and methods of the present invention include cationic lipids as described in U.S. Provisional Patent Application Ser. No. 62/864,818, filed on Jun. 21, 2019 and International Patent Publication WO 2020/257716, filed on Jun. 19, 2020, which are incorporated herein by reference. In some embodiments, the LNPs, compositions, pharmaceutical compositions and methods of the present invention include a cationic lipid having a compound structure according to the following formula:

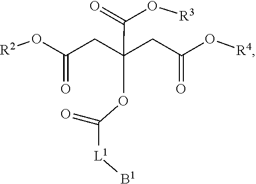

or a pharmaceutically acceptable salt thereof, wherein each of R², R³, and R⁴ is independently C₆-C₃₀ alkyl, C₆-C₃₀ alkenyl, or C₆-C₃₀ alkynyl; L¹ is C₁-C₃₀ alkylene; C₂-C₃₀ alkenylene; or C₂-C₃₀ alkynylene and B¹ is an ionizable nitrogen-containing group. In embodiments, L¹ is C₁-C₁₀ alkylene. In embodiments, L¹ is unsubstituted C₁-C₁₀ alkylene. In embodiments, L¹ is (CH₂)₂, (CH₂)₃, (CH₂)₄, or (CH₂)₅. In embodiments, L¹ is (CH₂), (CH₂)₆, (CH₂)₇, (CH₂)₈, (CH₂)₉, or (CH₂)₁₀. In embodiments, B¹ is independently NH₂, guanidine, amidine, a mono- or dialkylamine, 5- to 6-membered nitrogen-containing heterocycloalkyl, or 5- to 6-membered nitrogen-containing heteroaryl. In embodiments, B¹ is

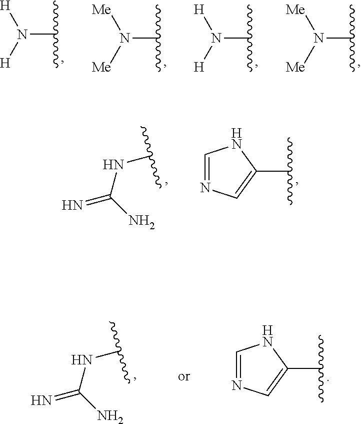

In embodiments, B¹ is

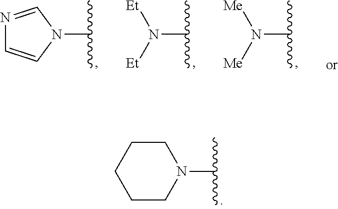

In embodiments, B¹ is

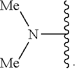 [SMILES: *N(C)C]

In embodiments, each of R², R³, and R⁴ is independently unsubstituted linear C₆-C₂₂ alkyl, unsubstituted linear C₆-C₂₂ alkenyl, unsubstituted linear C₆-C₂₂ alkynyl, unsubstituted branched C₆-C₂₂ alkyl, unsubstituted branched C₆-C₂₂ alkenyl, or unsubstituted branched C₆-C₂₂ alkynyl. In embodiments, each of R², R³, and R⁴ is unsubstituted C₆-C₂₂ alkyl. In embodiments, each of R², R³, and R⁴ is —C₆H₁₃, —C₇H₁₅, —C₈H₁₇, —C₉H₁₉, —C₁₀H₂₁, —C₁₁H₂₃, —C₁₂H₂₅, —C₁₃H₂₇, —C₁₄H₂₉, —C₁₅H₃₁, —C₁₆H₃₃, —C₁₇H₃₅, —C₁₈H₃₇, —C₁₉H₃₉, —C₂₀H₄₁, —C₂₁H₄₃, —C₂₂H₄₅, —C₂₃H₄₇, —C₂₄H₄₉, or —C₂₅H₅₁. In embodiments, each of R², R³, and R⁴ is independently C₆-C₁₂ alkyl substituted by —O(CO)R⁵ or —C(O)OR⁵, wherein R⁵ is unsubstituted C₆-C₁₄ alkyl. In embodiments, each of R², R³, and R⁴ is unsubstituted C₆-C₂₂ alkenyl. In embodiments, each of R², R³, and R⁴ is —(CH₂)₄CH═CH₂, —(CH₂)₅CH═CH₂, —(CH₂)₆CH═CH₂, —(CH₂)₇CH═CH₂, —(CH₂)₈CH═CH₂, —(CH₂)₉CH═CH₂, —(CH₂)₁₀CH═CH₂, —(CH₂)₁₁CH═CH₂, —(CH₂)₁₂CH═CH₂, —(CH₂)₁₃CH═CH₂, —(CH₂)₁₄CH═CH₂, —(CH₂)₁₅CH═CH₂, —(CH₂)₁₆CH═CH₂, —(CH₂)₁₇CH═CH₂, —(CH₂)₁₈CH═CH₂, —(CH₂)₇CH═CH(CH₂)₃CH₃, —(CH₂)₇CH═CH(CH₂)₅CH₃, —(CH₂)₄CH═CH(CH₂)₈CH₃, —(CH₂)₇CH═CH(CH₂)₇CH₃, —(CH₂)₆CH═CHCH₂CH═CH(CH₂)₄CH₃, —(CH₂)₇CH═CHCH₂CH═CH(CH₂)₄CH₃, —(CH₂)₇CH═CHCH₂CH═CHCH₂CH═CHCH₂CH₃, —(CH₂)₃CH═CHCH₂CH═CHCH₂CH═CHCH₂CH═CH(CH₂)₄CH₃, —(CH₂)₃CH═CHCH₂CH═CHCH₂CH═CHCH₂CH═CHCH₂CH═CHCH₂CH₃, —(CH₂)₁₁CH═CH(CH₂)₇CH₃, or —(CH₂)₂CH═CHCH₂CH═CHCH₂CH═CHCH₂CH═CHCH₂CH═CHCH₂CH═CHCH₂CH₃. In embodiments, said C₆-C₂₂ alkenyl is a monoalkenyl, a dienyl, or a trienyl. In embodiments, each of R², R³, and R⁴ is

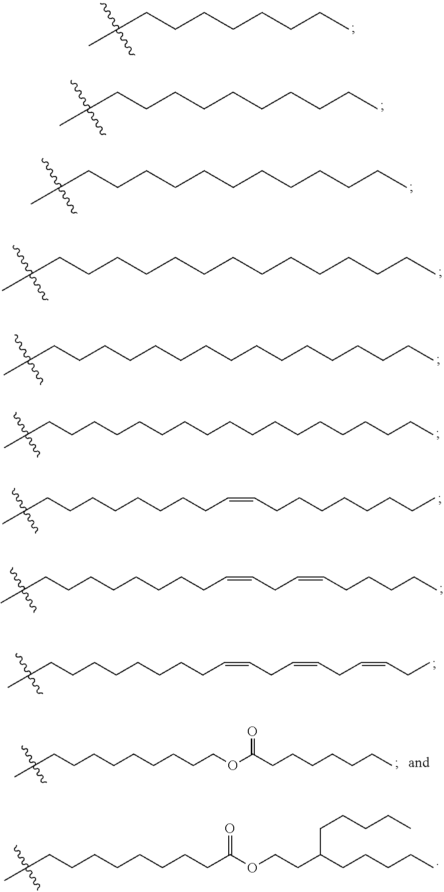

In some embodiments, the LNPs, compositions, pharmaceutical compositions and methods of the present invention include a cationic lipid having a compound structure of:

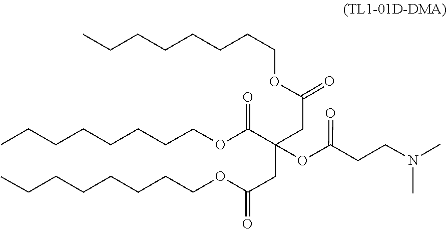 [SMILES: CCCCCCCCOC(=O)CC(CC(=O)OCCCCCCCC)(OC(=O)CCN(C)C)C(=O)OCCCCCCCC]

and pharmaceutically acceptable salts thereof. In some embodiments, the LNPs, compositions, pharmaceutical compositions and methods of the present invention include a cationic lipid having a compound structure of:

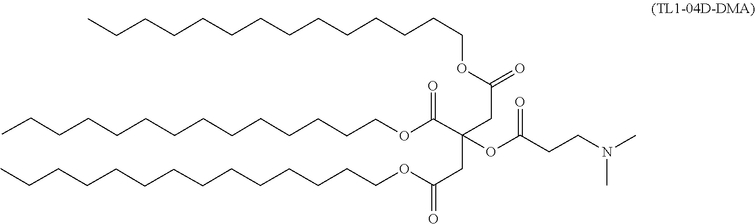 [SMILES: CCCCCCCCCCCCCCOC(=O)CC(CC(=O)OCCCCCCCCCCCCCC)(OC(=O)CCN(C)C)C(=O)OCCCCCCCCCCCCCC]

and pharmaceutically acceptable salts thereof. In some embodiments, the LNPs, compositions, pharmaceutical compositions and methods of the present invention include a cationic lipid having a compound structure of:

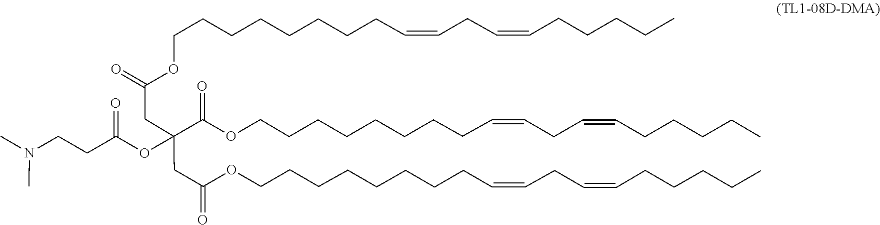 [SMILES: CCCCC/C=C\C/C=C\CCCCCCCCOC(=O)CC(CC(=O)OCCCCCCCC/C=C\C/C=C\CCCCC)(OC(=O)CCN(C)C)C(=O)OCCCCCCCC/C=C\C/C=C\CCCCC]

and pharmaceutically acceptable salts thereof. In some embodiments, the LNPs, compositions, pharmaceutical compositions and methods of the present invention include a cationic lipid having a compound structure of:

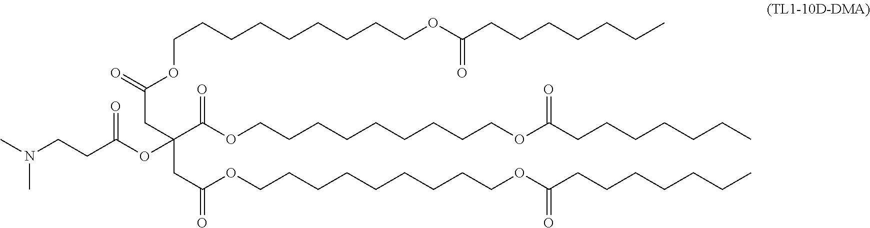 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

and pharmaceutically acceptable salts thereof. In some embodiments, the LNPs, compositions, pharmaceutical compositions and methods of the present invention include a cationic lipid of the following formula:

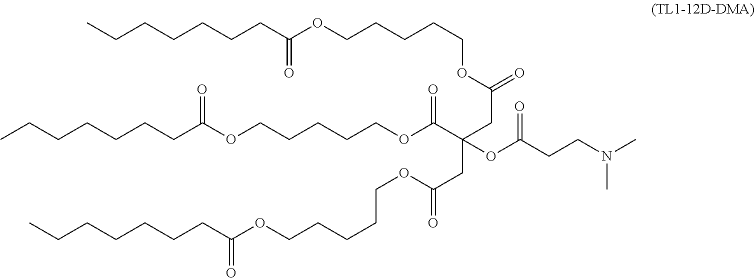 [SMILES: CCCCCCCC(=O)OCCCCCOC(=O)CC(CC(=O)OCCCCCOC(=O)CCCCCCC)(OC(=O)CCN(C)C)C(=O)OCCCCCOC(=O)CCCCCCC]

or a pharmaceutically acceptable salt thereof.

Other suitable cationic lipids for use in the LNPs, compositions, pharmaceutical compositions and methods of the present invention include cleavable cationic lipids as described in International Patent Publication WO 2012/170889, which is incorporated herein by reference. In some embodiments, the LNPs, compositions, pharmaceutical compositions and methods of the present invention include a cationic lipid of the following formula:

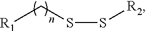

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

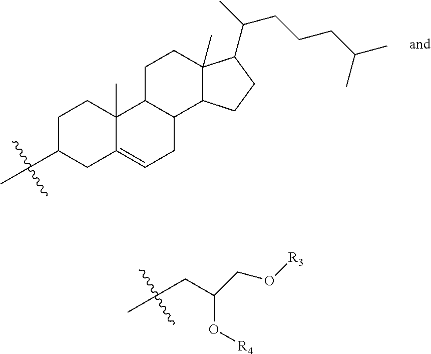

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In some embodiments, the LNPs, compositions, pharmaceutical compositions and methods of the present invention include a cationic lipid, “HGT4001”, having a compound structure of:

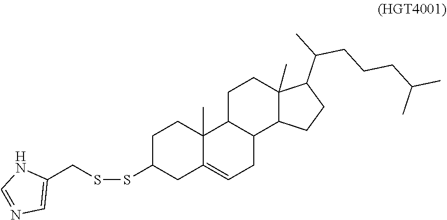 [SMILES: CC(C)CCCC(C)C1CCC2C3CC=C4CC(SSCc5cnc[nH]5)CCC4(C)C3CCC12C]

and pharmaceutically acceptable salts thereof. In some embodiments, the LNPs, compositions, pharmaceutical compositions and methods of the present invention include a cationic lipid, “HGT4002”, having a compound structure of:

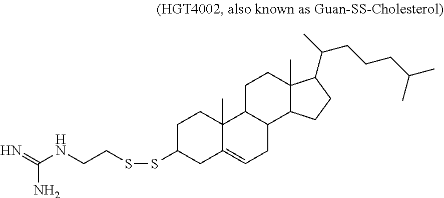 [SMILES: CC(C)CCCC(C)C1CCC2C3CC=C4CC(SSCCNC(=N)N)CCC4(C)C3CCC12C]

and pharmaceutically acceptable salts thereof. In some embodiments, the LNPs, compositions, pharmaceutical compositions and methods of the present invention include a cationic lipid, “HGT4003,” having a compound structure of:

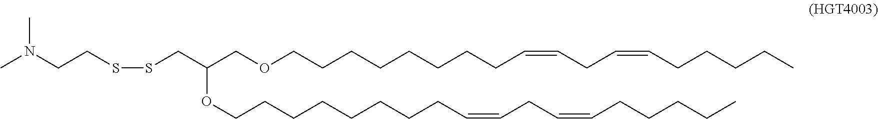 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In some embodiments, the LNPs, compositions, pharmaceutical compositions and methods of the present invention include a cationic lipid, “HGT4004,” having a compound structure of:

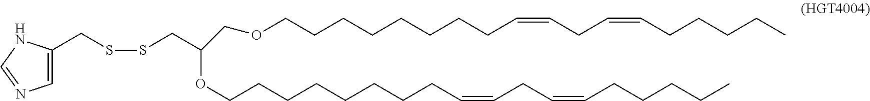 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In some embodiments, the LNPs, compositions, pharmaceutical compositions and methods of the present invention include a cationic lipid “HGT4005,” having a compound structure of:

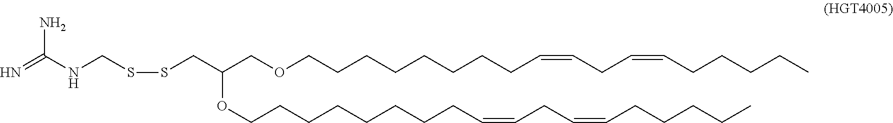 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the LNPs, compositions, pharmaceutical compositions and methods of the present invention include cleavable cationic lipids as described in International Patent Publication WO 2019/222424, and incorporated herein by reference. In some embodiments, the LNPs, compositions, pharmaceutical compositions and methods of the present invention include a cationic lipid that is any of general formulas or any of structures (1a)-(21a) and (1b)-(21b) and (22)-(237) described in International Patent Publication WO 2019/222424. In some embodiments, the LNPs, compositions, pharmaceutical compositions and methods of the present invention include a cationic lipid that has a structure according to Formula (I′),

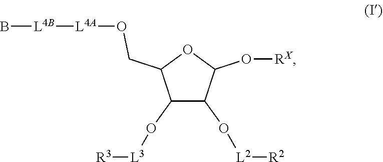

wherein:
R^{X} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In some embodiments, the LNPs, compositions, pharmaceutical compositions and methods of the present invention include a cationic lipid that is Compound (139) of International Patent Publication No. WO 2019/222424, having a compound structure of:

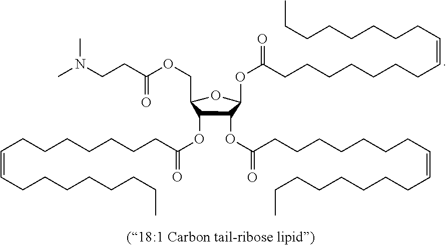

In some embodiments, the LNPs, compositions, pharmaceutical compositions and methods of the present invention include a cationic lipid that is RL3-DMA-07D having a compound structure of:

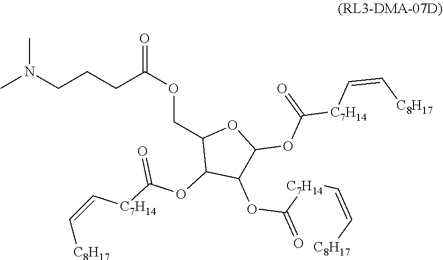

and pharmaceutically acceptable salts thereof.

In some embodiments, the LNPs, compositions, pharmaceutical compositions and methods of the present invention include a cationic lipid that is RL2-DMP-07D having a compound structure of:

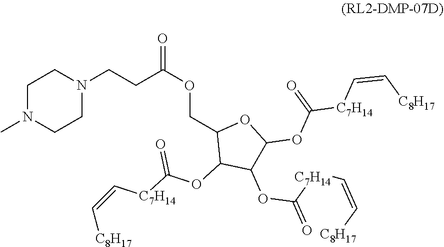

and pharmaceutically acceptable salts thereof.

In some embodiments, the LNPs, compositions, pharmaceutical compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the LNPs, compositions, pharmaceutical compositions and methods of the present invention include, for example, 5-carboxyspermylglycinedioctadecylamide (“DOGS”); 2,3-dioleyloxy-N-[2(spermine-carboxamido)ethyl]-N,N-dimethyl-l-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the LNPs, compositions, pharmaceutical compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethyl-1-(cis,cis-9′, 1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the LNPs, compositions, pharmaceutical compositions and methods of the present invention include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the LNPs, compositions, pharmaceutical composition, e.g., a lipid nanoparticle. In some embodiments, the LNPs, compositions, pharmaceutical compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the LNPs, compositions, pharmaceutical composition, e.g., a lipid nanoparticle.

### Non-Cationic Lipids

In some embodiments, the lipid nanoparticles contain one or more non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 1,2-dierucoyl-sn-glycero-3-phosphoethanolamine (DEPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof. In some embodiments, lipid nanoparticles suitable for use with the invention include DOPE as the non-cationic lipid component. In other embodiments, lipid nanoparticles suitable for use with the invention include DEPE as the non-cationic lipid component.

In some embodiments, a non-cationic lipid is a neutral lipid, i.e., a lipid that does not carry a net charge in the conditions under which the LNPs, compositions, pharmaceutical compositions are formulated and/or administered.

### Cholesterol-Based Lipids

In some embodiments, the lipid nanoparticle comprises one or more cholesterol-based lipids. For example, suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), as disclosed in International Patent Publication WO 2011/068810, which has the following structure:

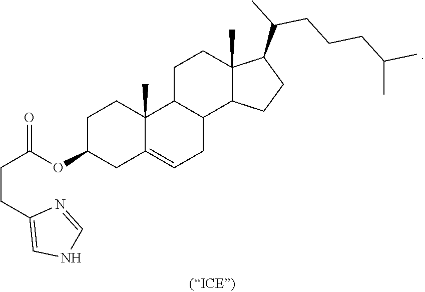 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

### PEG-Modified Lipids

In some embodiments, the lipid nanoparticle comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000](C₈ PEG-2000 ceramide) is also contemplated by the present invention, either alone or preferably in combination with other lipid pharmaceutical compositions together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid pharmaceutical composition to the target tissues, (Klibanov et al. (1990) FEBS Letters, 268 (1): 235-237), or they may be selected to rapidly exchange out of the pharmaceutical composition in vivo (see U.S. Pat. No. 5,885,613). Particularly useful exchangeable lipids are PEG-ceramides having shorter acyl chains (e.g., C₁₄ or Cis). Lipid nanoparticles suitable for use with the invention typically include a PEG-modified lipid such as 1,2-dimyristoyl-rac-glycero-3-methoxypolyethylene glycol-2000 (DMG-PEG2K).

In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio. For certain applications, such as pulmonary delivery, lipid nanoparticles in which the PEG-modified lipid component constitutes about 5% of the total lipids by molar ratio have been found to be particularly suitable.

### Exemplary Lipid Formulations

A typical LNP for use with the invention may be composed of one of the following combinations of a cationic lipid, a non-cationic lipid, a PEG-modified lipid and optionally cholesterol: cKK-E12, DOPE, cholesterol and DMG-PEG2K; cKK-E10, DOPE, cholesterol and DMG-PEG2K; OF-Deg-Lin, DOPE, cholesterol and DMG-PEG2K; OF-02, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; HGT4001, DOPE, cholesterol and DMG-PEG2K; HGT4002, DOPE, cholesterol and DMG-PEG2K; TL1-01D-DMA, DOPE, cholesterol and DMG-PEG2K; TL1-04D-DMA, DOPE, cholesterol and DMG-PEG2K; TL1-08D-DMA, DOPE, cholesterol and DMG-PEG2K; TL1-10D-DMA, DOPE, cholesterol and DMG-PEG2K; ICE, DOPE and DMG-PEG2K; HGT4001, DOPE and DMG-PEG2K; HGT4002, DOPE and DMG-PEG2K; SY-3-E14-DMAPr, DOPE, cholesterol and DMG-PEG2K; RL3-DMA-07D, DOPE, cholesterol and DMG-PEG2K; RL2-DMP-07D, DOPE, cholesterol and DMG-PEG2K; cHse-E-3-E10, DOPE, cholesterol and DMG-PEG2K; cHse-E-3-E12, DOPE, cholesterol and DMG-PEG2K; or cDD-TE-4-E12, DOPE, cholesterol and DMG-PEG2K. In specific embodiments, the LNP may be composed of SY-3-E14-DMAPr, DOPE, cholesterol and DMG-PEG2K. In other specific embodiments, the LNP may be composed of RL3-DMA-07D, DOPE, cholesterol and DMG-PEG2K. In yet other specific embodiments, the LNP may be composed of RL2-DMP-07D, DOPE, cholesterol and DMG-PEG2K. In yet other specific embodiments, the LNP may be composed of cHse-E-3-E10, DOPE, cholesterol and DMG-PEG2K. In yet other specific embodiments, the LNP may be composed of cHse-E-3-E12, DOPE, cholesterol and DMG-PEG2K. In yet other specific embodiments, the LNP may be composed of cDD-TE-4-E12, DOPE, cholesterol and DMG-PEG2K.

In some embodiments, cationic lipids (e.g., cKK-E12, cKK-E10, OF-Deg-Lin, OF-02, TL1-01D-DMA, TL1-04D-DMA, TL1-08D-DMA, TL1-10D-DMA, ICE, HGT4001, HGT4002, and/or SY-3-E14-DMAPr) constitute about 30-60% (e.g., about 30-55%, about 30-50%, about 30-45%, about 30-40%, about 35-50%, about 35-45%, or about 35-40%) of the lipid nanoparticle by molar ratio. In some embodiments, the percentage of cationic lipids (e.g., cKK-E12, cKK-E10, OF-Deg-Lin, OF-02, TL1-01D-DMA, TL1-04D-DMA, TL1-08D-DMA, TL1-10D-DMA, ICE, HGT4001, HGT4002, and/or SY-3-E14-DMAPr) is or greater than about 30%, about 35%, about 40%, about 45%, about 50%, about 55%, or about 60% of the lipid nanoparticle by molar ratio.

In some embodiments, the ratio of cationic lipid(s) to non-cationic lipid(s) to cholesterol-based lipid(s) to PEG-modified lipid(s) may be between about 30-60:15-35:0-30:1-15 by molar ratio. In some embodiments, the ratio of cationic lipid(s) to non-cationic lipid(s) to cholesterol-based lipid(s) to PEG-modified lipid(s) may be between about 30-60:25-35:20-30:1-15 by molar ratio. In some embodiments, the ratio of cationic lipid(s) to non-cationic lipid(s) to cholesterol-based lipid(s) to PEG-modified lipid(s) is approximately 40:30:20:10 by molar ratio. In some embodiments, the ratio of cationic lipid(s) to non-cationic lipid(s) to cholesterol-based lipid(s) to PEG-modified lipid(s) is approximately 40:30:25:5 by molar ratio. In some embodiments, the ratio of cationic lipid(s) to non-cationic lipid(s) to cholesterol-based lipid(s) to PEG-modified lipid(s) is approximately 40:32:25:3 by molar ratio. In some embodiments, the ratio of cationic lipid(s) to non-cationic lipid(s) to cholesterol-based lipid(s) to PEG-modified lipid(s) is approximately 50:25:20:5 by molar ratio. In some embodiments, the ratio of cationic lipid(s) to non-cationic lipid(s) to cholesterol-based lipid(s) to PEG-modified lipid(s) is approximately 50:15:30:5 by molar ratio.

In some embodiments, lipid nanoparticles suitable for use with the invention comprise four lipid component: a cationic lipid (typically an ionizable cationic lipid such as SY-3-E14-DMAPr), a non-cationic lipid (e.g., DOPE), cholesterol and a PEG-modified lipid (e.g., DMG-PEG2K) at a molar ratio of about 40-50 (cationic lipid):15-30 (non-cationic lipid):25-30 (cholesterol):5 (PEG-modified lipid). In one specific embodiment, the molar ratios are 50:15:30:5. In another specific embodiment, the molar ratios are 40:30:25:5. The N/P ratio typically is about 4.

In typical three-component lipid nanoparticles suitable for use with the invention, the molar ratio of cationic lipid to non-cationic lipid to PEG-modified lipid may be between about 55-65:30-40:1-15, respectively. In some embodiments, a molar ratio of cationic lipid (e.g., a sterol-based lipid) to non-cationic lipid (e.g., DOPE or DEPE) to PEG-modified lipid (e.g., DMG-PEG2K) of 60:35:5 is particularly suitable, e.g., for pulmonary delivery of lipid nanoparticles via nebulization. In one specific embodiment, a suitable three-component lipid nanoparticle comprises HGT4002, DOPE and DMG-PEG2K. In another specific embodiment, a suitable three-component lipid nanoparticle comprises ICE, DOPE and DMG-PEG2K. The N/P ratio typically is about 4.

### Polymers

In some embodiments, a suitable LNP delivery vehicle is formulated using a polymer as a carrier, alone or in combination with other carriers including various lipids described herein. Thus, in some embodiments, LNPs, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

### Compositions

The invention provides compositions comprising an mRNA of the invention. In some embodiments, a composition in accordance with the invention comprises an mRNA of the invention at a concentration ranging from about 0.5 mg/mL to about 1.0 mg/mL. In some embodiments, the mRNA is at a concentration of at least 0.5 mg/mL. In some embodiments, the mRNA is at a concentration of at least 0.6 mg/mL. In some embodiments, the mRNA is at a concentration of at least 0.7 mg/mL. In some embodiments, the mRNA is at a concentration of at least 0.8 mg/mL. In some embodiments, the mRNA is at a concentration of at least 0.9 mg/mL. In some embodiments, the mRNA is at a concentration of at least 1.0 mg/mL. In a typical embodiment, the mRNA is at a concentration of about 0.6 mg/mL to about 0.8 mg/mL.

Typically, the mRNA in the composition is encapsulated in LNPs. To stabilize the mRNA or the LNPs encapsulating it, or to enhance in vivo expression of the mRNAs, the compositions of the invention may be formulated with one or more carrier, stabilizing reagent or other excipients. Such compositions may be pharmaceutical compositions, and as such they may include one more or more pharmaceutically acceptable excipients. The one or more pharmaceutically acceptable excepients may be selected from a buffer, a sugar, a salt, a surfactant or combinations thereof.

### Pharmaceutically Acceptable Excipients

In some embodiments, the pharmaceutical composition is formulated with a diluent. In some embodiments, the diluent is selected from a group consisting of ethylene glycol, glycerol, propylene glycol, sucrose, trehalose, or combinations thereof. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

In some embodiments, the LNPs are suspended in an aqueous solution comprising a disaccharide. Suitable disaccharides for use with the invention include trehalose and sucrose. For example, in some embodiments, the LNPs are suspended in an aqueous solution comprising trehalose, e.g., 10% (w/v) trehalose in water. In other embodiments, LNPs are suspended in an aqueous solution comprising sucrose, e.g., 10% (w/v) sucrose in water.

In some embodiments, the aqueous solution further comprises a buffer, a salt, a surfactant or combinations thereof.

In some embodiments, the salt is selected from the group consisting of NaCl, KCl, and CaCl₂). Accordingly, in some embodiments, the salt is NaCl. In some embodiments, the salt is KCl. In some embodiments, the salt is CaCl₂).

In some embodiments, the buffer is selected from the group consisting of a phosphate buffer, a citrate buffer, an imidazole buffer, a histidine buffer, and a Good's buffer. Accordingly, in some embodiments, the buffer is a phosphate buffer. In some embodiments, the buffer is a citrate buffer. In some embodiments, the buffer is an imidazole buffer. In some embodiments, the buffer is a histidine buffer. In some embodiments, the buffer is a Good's buffer. In some embodiments, the Good's buffer is a Tris buffer or HEPES buffer.

In particular embodiments, the buffer is a phosphate buffer (e.g., a citrate-phosphate buffer), a Tris buffer, or an imidazole buffer.

In some embodiments, the composition comprises a buffer and a salt (typically in addition to a suitable diluent such as a disaccharide or optionally a propylene glycol). In some embodiments, the total concentration of the buffer and the salt is selected from about 40 mM Tris buffer and about 75-125 mM NaCl, about 50 mM Tris buffer and about 50 mM-100 mM NaCl, about 100 mM Tris buffer and about 100 mM-200 mM NaCl, about 40 mM imidazole and about 100 mM-125 mM NaCl, and about 50 mM imidazole and 75 mM-100 mM NaCl.

### Therapeutically Effective Amount

The mRNA in accordance with the invention is provided in a therapeutically effective amount in the pharmaceutical compositions provided herein. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating or preventing an infection in a subject caused by a virus which uses human ACE2 protein for cellular entry). For example, a therapeutically effective amount may be an amount sufficient to neutralize a virus which uses human ACE2 protein for cellular entry in a subject, using a pharmaceutical composition of the invention.

### Dry Powder Formulations

The invention also provides dry powder formulations comprising a plurality of spray-dried particles comprising the LNPs encapsulating an mRNA of the invention. Typically, a dry powder formulation suitable for use with the invention includes one or more polymers. Additionally, it may include one or more other excipients, e.g., one or more sugars or sugar alcohols and/or one or more surfactants. In a typical embodiment of the invention, a dry powder formulation comprises LNPs encapsulating an mRNA of the invention, one or more polymers (e.g., polymethacrylate-based polymer such as Eudragit EPO), one or more sugars or sugar alcohols or combinations thereof (e.g., mannitol, or mannitol and lactose or mannitol and trehalose), and optionally one or more surfactants (e.g., a poloxamer such as poloxamer 407). Providing the LNPs encapsulating an mRNA as a dry powder formulation in accordance with the invention is advantageous because such formulations are stable and the spray-dried particles are inhalable, i.e., the can be directly administered to a subject.

Intranasal, intratracheal, and/or inhaled routes of administrations are particularly suitable for the dry powder formulations described herein, as they facilitate delivery of the LNP-encapsulated mRNAs of the invention to the airway epithelial cells which are most susceptible to infection by viruses which use the human ACE2 protein for cellular entry, such as SARS-CoV-2. For example, aerosols comprising dry powder formulations of the present invention can be inhaled (for nasal, tracheal, or bronchial delivery).

In some embodiments, the dry powder formulation is administered by inhalation. In particular embodiments, the spray-dried particles are reconstituted prior to administration and the reconstituted suspension of LNPs is administered via nebulization.

### Polymer

Various polymers may be used in a dry powder formulation in accordance with the invention. Typically, suitable polymers have low toxicity and are well tolerated over a wide range of concentrations. In some embodiments, suitable polymers are positively charged. Exemplary polymers include, but are not limited to, chitosan, polyesters, polyurethanes, polycarbonates, poly(lactic acid) (PLA), poly(lactic-co-glycolic acid) (PLGA), poly(q-caprolactone (PCL), poly amido amines, poly(hydroxyalkyl L-asparagine), poly(hydroxyalkyl L-glutamine), poly(2-alkyloxazoline) acrylates, modified acrylates and methacrylate based polymers, poly-N-(2-hydroxyl-propyl)methacrylamide, poly-2-(methacryloyloxy)ethyl phosphorylcholines, poly(2-(methacryloyloxy)ethyl phosphorylcholine), and poly(dimethylaminoethyl methylacrylate) (pDMAEMA).

In a typical embodiment, a dry formulation in accordance with the invention comprises one or more polymethacrylate-based polymer. A particularly suitable polymethacrylate-based polymer for use with the invention is Eudragit EPO.

### Sugars or Sugar Alcohols

Exemplary sugars or sugar alcohols suitable for use with a dry powder formulation in accordance with the invention are monosaccharides, disaccharides and polysaccharides, selected from a group consisting of glucose, fructose, galactose, mannose, sorbose, lactose, sucrose, cellobiose, trehalose, raffinose, starch, dextran, maltodextrin, cyclodextrins, inulin, xylitol, sorbitol, lactitol, and mannitol.

In some embodiments, a suitable sugar or sugar alcohol is lactose and/or mannitol. In some embodiments, a suitable sugar is mannitol. In some embodiments, the mannitol is added at a concentration of about 1-10%. In some embodiments, the mannitol is added at a concentration of about 2-10%. In some embodiments, the mannitol is added at a concentration of about 3-10%. In some embodiments, the mannitol is added at a concentration of about 4-10%. In some embodiments, the mannitol is added at a concentration of about 5-10%.

In some embodiments, a suitable sugar is trehalose. In some embodiments, both mannitol and trehalose are added.

### Surfactants

In some embodiments, a dry formulation in accordance with the invention further comprises one or more surfactants. Surfactants increase the surface tension of a composition. In some embodiments, the surfactants used in spray-drying mRNA lipid compositions are selected from a group consisting of CHAPS (3-[(3-Cholamidopropyl)dimethylammonio]-1-propanesulfonate), phospholipids, phosphatidylserine, phosphatidylethanolamine, phosphatidylcholine, sphingomyelins, octaethylene glycol monododecyl ether, pentaethylene glycol monododecyl ether, Triton X-100, Cocamide monoethanolamine, Cocamide diethanolamine, Glycerol monostearate, Glycerol monolaurate, Sorbitan moonolaureate, Sorbitan monostearate, Tween 20, Tween 40, Tween 60, Tween 80, Alkyl polyglucosides, and poloxamers.

In a particular embodiment, the surfactant is a poloxamer (e.g., poloxamer 407).

### Methods of Treatment

### Methods of Treatment

The mRNAs of the invention, and compositions comprising these mRNAs, are for use in therapy, in particular for use in the therapeutic methods disclosed herein. Without wishing to be bound by any particular theory, the inventors believe that the polypeptides encoding the extracellular domain of human ACE2 protein encoded by the mRNAs of the invention are able to bind to and effectively neutralize viruses which rely on the ACE2 receptor for cellular entry, including future emerging viruses or virus strains (typically referred to as “variants”). The compositions, pharmaceutical compositions, and/or dry powder formulations of the invention can be administered to a subject by various routes, as further described below.

### Subjects to be Treated

The mRNAs of the invention, and compositions comprising them, can be used to treat subjects infected with a virus which uses the human ACE2 protein for cellular entry. In specific embodiments, the subject has, or is diagnosed with, coronavirus disease (COVID-19). COVID-19 can be mild or severe, as described above. The compositions and pharmaceutical compositions of the invention can be administered to such subjects, to neutralize SARS-CoV-2 viral particles present in the subject and reduce the overall viral load of SARS-CoV-2. As the viral load of SARS-CoV-2 predicts COVID-19 mortality, it is therefore advantageous to reduce the viral load of SARS-CoV-2 by administering the compositions and pharmaceutical compositions of the invention. A diagnosis of COVID-19 can be confirmed, for example, with a diagnostic test for COVID-19. For example, viral tests are used to test samples obtained from a subject to determine whether they are currently infected with SARS-CoV-2. Suitable viral tests include nucleic acid amplification tests which detect viral nucleic acids in samples obtained from a subject (e.g. PCR tests), antigen tests which detect SARS-CoV-2 viral proteins in such samples (e.g. lateral flow immunoassays), or genomic sequencing which detects and identifies viral nucleic acids in such samples.

Severe COVID-19 may comprise symptoms including acute pneumonia, respiratory distress, or other organ damage. Severe COVID-19 typically leads to respiratory disease in subjects, as described above. In particular, subjects with lung damage or who have difficulty breathing due to COVID-19 may benefit from intravenous administration of the compositions and/or pharmaceutical compositions of the invention (e.g. compositions or pharmaceutical compositions comprising LNP-encapsulated mRNAs comprising an optimized nucleotide sequence encoding a polypeptide comprising the extracellular domain of human ACE2 protein and an immunoglobulin Fc region, and optionally a localization sequence). Administration of the LNPs encapsulating the mRNAs of the invention by inhalation of an aerosol may be a less feasible delivery route for such subjects with severe COVID-19.

In situations where the subject is particularly vulnerable to COVID-19 (e.g. the subject is immunocompromised or immunosuppressed), has not been vaccinated against COVID-19 or against SARS-CoV-2 infection, or cannot be safely vaccinated against COVID-19 or against SARS-CoV-2 infection, there is a pressing need for other therapeutic options which offer protection against infection. The compositions, pharmaceutical compositions, and dry powder formulations of the present invention are particularly useful in treating COVID-19 or providing protection against SARS-CoV-2 infection in such subjects.

Older adults are at higher risk for severe illness from COVID-19 due to a variety of factors, for example weakening of the immune response with age which results in a decreased ability to control viral load of SARS-CoV-2, which predicts COVID-19 mortality. For example, the compositions, pharmaceutical compositions, and dry powder formulations of the present invention are particularly useful in preventing infection with SARS-CoV-2 or treating COVID-19 in subjects, wherein the subject is 50 years of age and over, 55 years of age and over, 60 years of age and over, 65 years of age and over, 65 years of age and over, 70 years of age and over, 75 years of age and over, 80 years of age and over, 85 years of age and over, 90 years of age and over, or 95 years of age and over. Such subjects may not be able to suppress and eliminate SARS-CoV-2, the causative agent of COVID-19, without further therapeutic intervention.

### Intravenous Administration

Subjects with lung damage or who have difficulty breathing due to COVID-19 may benefit from intravenous administration of an mRNA of the invention, as administration of the mRNA by inhalation of an aerosol may be a less feasible delivery route for such subjects. In addition, without wishing to be bound by any particular theory, the inventors believe that sustained levels of the mRNA-expressed polypeptides in serum may provide effective protection against systemic viral infection by neutralizing the viral particles present in circulation and preventing or reducing infection of distal organs and tissues accessible via the systemic circulation, thereby providing a broader therapeutic benefit than achievable by localized administration of the mRNAs of the invention.

Accordingly, in a particular embodiment, the invention provides a method of treating or preventing an infection in a subject caused by a virus which uses human ACE2 protein for cellular entry, wherein the method comprises administering a composition comprising an mRNA of the invention to the subject intravenously. Typically, the mRNA is encapsulated in a lipid nanoparticle (LNP) to protect it against degradation while in circulation and for efficient delivery of the mRNA into cells for expression of the mRNA-encoded polypeptide. In particular embodiments, a LNP encapsulating an mRNA of the invention which is administered to a subject intravenously may comprise a cationic lipid selected from: RL3-DMA-07D, RL2-DMP-07D, cHse-E-3-E10, cHse-E-3-E12, and cDD-TE-4-E12.

In such embodiments, the polypeptide encoded by the mRNA of the invention usually comprises an immunoglobulin Fc region, typically linked to the C-terminus of the extracellular domain of human ACE2 protein, or portion thereof, which binds to a viral surface protein. As explained above, the presence of an immunoglobulin Fc region within the polypeptides encoded by the mRNA of the invention increases stability of the polypeptides, prolongs the half-life, and helps to sustain the levels of the mRNA-expressed polypeptides in serum. In some embodiments, the polypeptide encoded by the mRNA of the invention may also comprise a C-terminal localization sequence to enhance translocation across the epithelia of the airway.

### Aerosol Administration

Delivery of an mRNA of the invention to epithelial cells lining both the surfaces of the lower airways and the surfaces of the upper airway is considered to be beneficial to subjects, as ACE2 protein receptors are present on the epithelial cells in both the lower and upper airways which can be used by viruses such as SARS-CoV-2 for cellular entry.

Accordingly, in some embodiments, the therapeutic methods described herein comprise administering an mRNA of the invention as an aerosol. Typically, the mRNA is encapsulated in a lipid nanoparticle (LNP) to protect it against degradation by nucleases which are present in the airways. The aerosol may be administered by intranasal administration (e.g. by intranasal spray). Alternatively, the aerosol may be administered by pulmonary administration. For example, administration to the lower airways deep in the lung is achieved most effectively by nebulization. Administration to the lower airways of the lung can also be achieved by administering a composition of the invention by a metered-dose inhaler. Administration of a composition of the invention can be performed in an outpatient setting, for example in situations where the subjects do not require admission into a hospital for emergency care, which facilitates the ease of treatment comprising administering a composition of the invention to a subject.

### Nebulization

Inhaled aerosol droplets of a particle size of less than 8 μm (e.g., 1-5 μm) can penetrate into the narrow branches of the lower airways. Aerosol droplets with a larger diameter are typically absorbed by the epithelial cells lining the oral cavity and upper airway, and are unlikely to reach the lower airway epithelium and the deep alveolar lung tissue. Accordingly, in particular in the context of pulmonary delivery to the lung epithelium, methods that comprise administering an mRNA of the invention as an aerosol may include steps of generating droplets of a particle size of less than 8 μm (e.g., 1-5 μm), typically by nebulization of a composition of the invention, e.g. by using a nebulizer that is suitable for use with the compositions of the invention.

In a specific embodiment, a composition including the mRNA of the invention is nebulized to generate nebulized particles for inhalation by the subject. Nebulized particles for inhalation by a subject typically have an average size less than 8 μm. In some embodiments, the nebulized particles for inhalation by a subject have an average size between approximately 1-8 μm. In particular embodiments, the nebulized particles for inhalation by a subject have an average size between approximately 1-5 μm. In specific embodiments, the mean particle size of the nebulized composition of the invention is between about 4 μm and 6 μm, e.g., about 4 μm, about 4.5 μm, about 5 μm, about 5.5 μm, or about 6 μm.

Particle size in an aerosol is commonly described in reference to the Mass Median Aerodynamic Diameter (MMAD). MMAD, together with the geometric standard deviation (GSD), describes the particle size distribution of any aerosol statistically, based on the weight and size of the particles. Means of calculating the MMAD of an aerosol are well known in the art. For example, the MMAD output of a nebulizer using a composition of the invention can be determined using a Next Generation Impactor. Another parameter to describe particle size in an aerosol is the Volume Median Diameter (VMD). VMD also describes the particle size distribution of an aerosol based on the volume of the particles. Means of calculating the VMD of an aerosol are well known in the art. A specific method used for determining the VMD is laser diffraction, which is used herein to measure the VMD of a composition of the invention (see, e.g., Clark, 1995, Int J Pharm. 115:69-78).

Accordingly, in some embodiments, nebulization in accordance with the invention is performed to generate a Fine Particle Fraction (FPF), which is defined as the proportion of particles in an aerosol which have an MMAD or a VMD smaller than a specified value. In one specific embodiment, the FPF of a nebulized composition of the invention with a particle size <5 μm is at least about 30%, more typically at least about 40%, e.g., at least about 50%, more typically at least about 60%. In another specific embodiment, nebulization is performed in such a manner that the mean respirable emitted dose (i.e., the percentage of FPF with a particle size <5 μm; e.g., as determined by next generation impactor with 15 L/min extraction) is at least about 30% of the emitted dose, e.g., at least about 31%, at least about 32%, at least about 33%, at least about 34%, or at least about 35% the emitted dose. In yet another specific embodiment, nebulization is performed in such a manner that the mean respirable delivered dose (i.e., the percentage of FPF with a particle size <5 μm; e.g., as determined by next generation impactor with 15 L/min extraction) is at least about 15% of the emitted dose, e.g. at least 16% or 16.5% of the emitted dose.

In some embodiments, nebulization is performed with a nebulizer. One type of nebulizer is a jet nebulizer, which comprises tubing connected to a compressor, which causes compressed air or oxygen to flow at a high velocity through a liquid medicine to turn it into an aerosol, which is then inhaled by the subject. Another type of nebulizer is the ultrasonic wave nebulizer, which comprises an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Exemplary ultrasonic wave nebulizers are the Omron NE-U17 and the Beurer Nebulizer IH30. A third type of nebulizer comprises vibrating mesh technology (VMT). A VMT nebulizer typically comprises a mesh/membrane with 1000-7000 holes that vibrates at the top of a liquid reservoir and thereby pressures out a mist of very fine aerosol droplets through the holes in the mesh/membrane. Exemplary VMT nebulizers include eFlow (PARI Medical Ltd.), i-Neb (Respironics Respiratory Drug Delivery Ltd), Nebulizer IH50 (Beurer Ltd.), AeroNeb Go (Aerogen Ltd.), InnoSpire Go (Respironics Respiratory Drug Delivery Ltd), Mesh Nebulizer (Shenzhen Homed Medical Device Co, Ltd), Portable Nebulizer (Microbase Technology Corporation) and Airworks (Convexity Scientific LLC). In some embodiments, the mesh or membrane of the VMT nebulizer is made to vibrate by a piezoelectric element. In some embodiments, the mesh or membrane of the VMT nebulizer is made to vibrate by ultrasound.

VMT nebulizers have been found to be particularly suitable for practicing the invention because they do not affect the mRNA integrity of the mRNA encapsulated within LNPs of the invention, present in a composition. Typically, at least about 60%, e.g., at least about 65% or at least about 70%, of the mRNA in the compositions of the invention maintains its integrity after nebulization.

In some embodiments, nebulization is continuous during inhalation and exhalation. More typically, nebulization is breath-actuated. Suitable nebulizers for use with the invention have nebulization rate greater than 0.2 mL/min. In some embodiments, the nebulization rate is greater than 0.25 mL/min. In other embodiment, the nebulization rate is greater than 0.3 mL/min. In certain embodiments, the nebulization rate is greater than 0.45 mL/min. In a typical embodiment, the nebulization rate ranges between 0.2 mL/min and 0.5 mL/min.

### Timing of Administration

In some embodiments, the therapeutic methods provided herein comprise administering a composition of the invention before and/or after exposure, or suspected exposure, to a virus which uses human ACE2 protein for cellular entry in a subject. For example, a composition of the invention may be administered to a subject after infection with such a virus. In some embodiments, a composition of the invention is administered to a subject after exposure, or suspected exposure, to a virus which uses human ACE2 protein for cellular entry in a subject (such as SARS-CoV-2), and before the onset of at least one symptom of coronavirus disease 19 (COVID-19).

Administration of a composition of the invention to a subject after infection with, exposure to, or suspected exposure to a virus which uses human ACE2 protein for cellular entry may reduce the viral load in the subject, mitigate the existing infection, and prevent further infection and spread of the viral infection throughout the subject. Reducing the viral load in an infected subject by administering the compositions of the invention may also reduce the likelihood that the infected subject will lead to secondary transmission of said virus to other subjects.

In some embodiments, the therapeutic methods described herein comprise administering a composition of the invention once. In other embodiments, the composition is administered at least once. For example, the composition may be administered at least twice. Alternatively, the composition may be administered at least three times. The interval between administrations may be 1 day, 2 days, 3 days, 4 days, 5 days, 6 days, 7 days, or 8 days.

For example, a single administration of an mRNA of the invention, e.g. encapsulated in an LNP and delivered as an aerosol, may confer protection against infection by SARS-CoV-2 for several days (e.g. 3 to 4 days post administration), after which the protective effect may wane over the following days. Accordingly, in some embodiments, the interval between administrations may be 3-4 days, as post-exposure prophylaxis.

A single administration of the mRNA may provide protection during the crucial incubation period of COVID-19 (before onset of symptoms) which lasts 5-6 days, on average. Accordingly, in some embodiments, the therapeutic methods described herein comprise administering a composition of the invention once, e.g., as post-exposure prophylaxis, e.g. for use in the prevention of a COVID-19 infection.

Administration of an mRNA of the invention may also be effective in an acute disease setting where a rapid reduction in viral titres in the lung may buy time for a subject to recover and mount an effective immune response on their own, e.g. in form of virus-neutralizing antibodies. Accordingly, in some embodiments, the therapeutic methods described herein comprise administering a composition of the invention once or more than once, as needed, to treat a subject suffering from an acute infection with a virus which uses the human ACE2 protein for cellular entry (e.g. SARS-CoV-2).

As demonstrated in a mouse model in the Examples, the sustained duration of detectable expression of mRNA-derived polypeptides comprising the extracellular domain of human ACE2 protein and an immunoglobulin Fc region in the airway lumen and in the general circulation, up to 8 days following intravenous administration, is advantageous in conferring protection against infection by viruses which use the human ACE2 receptor for cellular entry. Accordingly, in some embodiment, an mRNAs encoding such polypeptides (e.g., the polypeptide encoded by SEQ ID NO: 3 or SEQ ID NO: 8) is administered as a single intravenous administration to confer protection against infection (e.g., by SARS-CoV-2). Typically, the mRNA is encapsulated in an LNP.

### EXAMPLES

The following examples are included for illustrative purposes only and are not intended to limit the scope of the invention.

### Example 1. Generating Optimized Nucleotide Sequences

This example illustrates a process that generates nucleotide sequences optimized to yield full-length transcripts during in vitro synthesis and high levels of expression of the encoded protein in vivo.

The process combines the codon optimization method of FIG. 1A with a sequence of filtering steps illustrated in FIG. 1B to generate a list of optimized nucleotide sequences. Specifically, as illustrated in FIG. 1A, the process receives an amino acid sequence of interest and a first codon usage table which reflects the frequency of each codon in a given organism (namely human codon usage preferences in the context of the present example). The process then removes codons from the first codon usage table if they are associated with a codon usage frequency which is less than a threshold frequency (10%). The codon usage frequencies of the codons not removed in the first step are normalized to generate a normalized codon usage table.

Normalizing the codon usage table involves re-distributing the usage frequency value for each removed codon; the usage frequency for a certain removed codon is added to the usage frequencies of the other codons with which the removed codon shares an amino acid. In this example, the re-distribution is proportional to the magnitude of the usage frequencies of the codons not removed from the table. The process uses the normalized codon usage table to generate a list of optimized nucleotide sequences. Each of the optimized nucleotide sequences encode the amino acid sequence of interest.

As illustrated in FIG. 1B, the list of optimized nucleotide sequences is further processed by applying a motif screen filter, guanine-cytosine (GC) content analysis filter, and codon adaptation index (CAI) analysis filter, in that order, to generate an updated list of optimized nucleotide sequences.

The subsequent examples confirm that this process results in nucleotide sequences encoding the amino acid sequence of interest that are optimized to yield full-length transcripts during in vitro synthesis and high levels of expression of the encoded protein in vivo.

### Example 2. Expression and Detection of Sequence-Optimized mRNAs Encoding Polypeptides Comprising the Extracellular Domain of Human ACE2 Protein

This example demonstrates that mRNAs encoding a polypeptide comprising the extracellular domain of human angiotensin-converting enzyme 2 (ACE2) protein are successfully expressed, processed and secreted by cultured cells, following transfection. This example also confirms that neither amino acid substitutions made to increase the binding affinity for SARS-CoV-2 spike (S) glycoprotein nor the sequence optimization steps described in Example 1 interfere with the expression or secretion of the encoded polypeptides.

The extracellular domain of human ACE2 protein comprises amino acid residues 1-740 of the full-length human ACE2 protein, as illustrated in FIG. 2. The mRNAs with optimized nucleotide sequences were designed to encode the extracellular domain of ACE2 only, excluding the transmembrane region and short intracellular region. This approach was chosen to ensure that viruses which use the ACE2 receptor present on the cell membrane for cellular entry cannot use the encoded polypeptides to enter and infect cells expressing the sequence-optimized mRNAs.

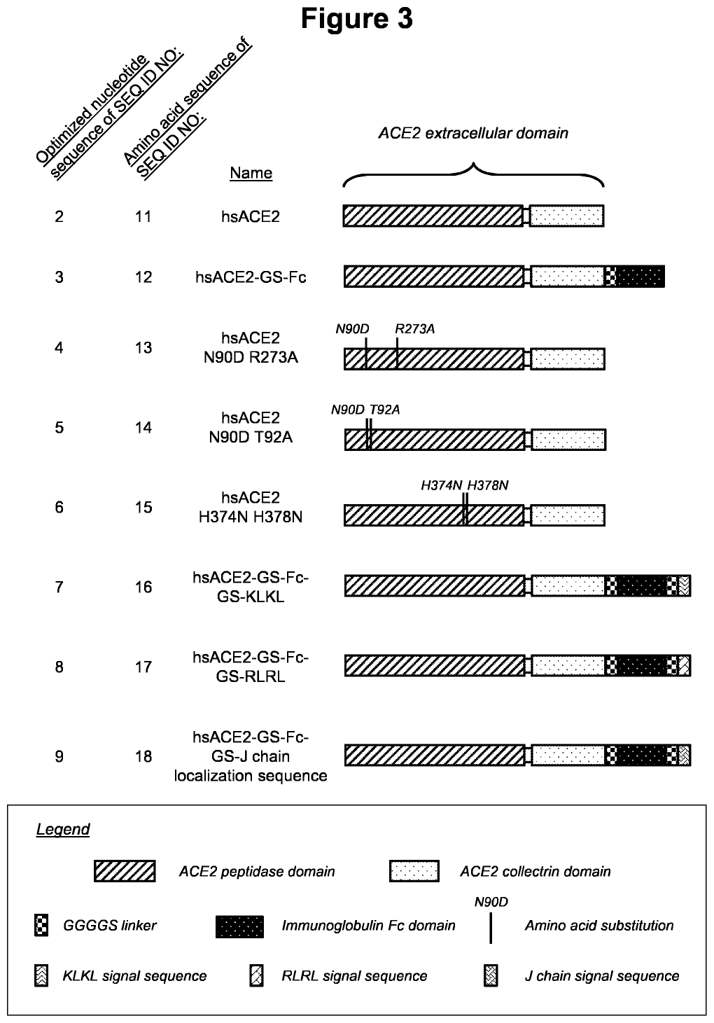

Various exemplary optimized nucleotide sequences encoding the extracellular domain of human ACE2 were designed, as schematically illustrated in FIG. 3. The mRNA nucleotide sequences are described in Table 3 and the polypeptide amino acid sequences are described in Table 2. The optimized nucleotide sequences were generated according to a codon optimization method, as illustrated in FIGS. 1A and 1B and described in Example 1. The polypeptides encoded by optimized nucleotide sequences SEQ ID NO: 4, SEQ ID NO: 5, and SEQ ID NO: 6 contain amino acid substitutions N90D and R273A, N90D and T92A, and H374N and H378N, respectively, relative to a naturally occurring human ACE2 protein encoded by the amino acid sequence of SEQ ID NO: 10. The amino acid substitutions were chosen because they have been shown to increase the binding affinity of the human ACE2 protein for SARS-CoV-2 spike (S) glycoprotein. ACE2 variants comprising these amino acid substitutions may have increased efficacy in neutralizing SARS-CoV-2 as ‘decoy receptors’ because they may be able to out-compete the endogenous ACE2 receptors for binding of SARS-CoV-2. The R273A mutation of the polypeptide has been shown to abolish the catalytic activity of soluble ACE2 (Ameratunga et al., (2020), New Zealand Medical Journal, Vol. 133, No. 1515). A decoy receptor with catalytic activity may not be desirable, when expressed at high levels in vivo. The N90D mutation has been shown to reduce N-glycosylation and increase the binding affinity of the ACE2 receptor to the SARS-CoV-2 spike (S) glycoprotein in its pre-fusion conformation (Ameratunga et al. (2020) New Zealand Medical Journal, Vol. 133, No. 1515; Chan et al. (2020) Science, Vol. 369, Issue 6508, pp. 1261-1265). The T92A mutation also affects the glycosylation of ACE2 and increases the binding affinity of the ACE2 receptor to the SARS-CoV-2 spike (S) glycoprotein. Finally, the H374 and H378 residues normally coordinate a Zn²⁺ ion necessary for enzymatic activity of ACE2, and the H374N and H378N mutations also increase the binding affinity of the ACE2 receptor to the SARS-CoV-2 spike (S) glycoprotein (Glasgow et al. (2020), PNAS Vol. 117 (45), pp. 28046-28055).

mRNA was synthesized by in vitro transcription (IVT) from DNA vectors encoding mRNAs comprising the optimized nucleotide sequences described above, and used to transfect HEK293 cells in culture. For transfection of cultured cells, 1 million HEK293 cells per well were plated in a 6 well cell culture plate. 150 μL OptiMEM Reduced Serum Medium was added to a 1.5 mL Eppendorf tube, along with 1 μg mRNA and 2.5 μL Lipofectamine 2000 for complexation of the mRNAs to the transfection reagent. Each tube was gently mixed on a Vortex and spun briefly in a microcentrifuge to collect the contents. The complexes were incubated for 10±2 minutes at room temperature. Then, the entire complex volume was carefully added to each well of a 6 well plate, so as not to disturb the HEK293 cell monolayer (1 million cells per well). The cells were returned to a 37° C. incubator and incubated for 18±2 hours prior to harvesting.

A 2.3 mL sample of the HEK293 cell culture medium (supernatant) was removed from each well. After removing the culture medium, the cell lysate samples from each well were harvested by removing the culture medium and adding 500 μL of CelLytic M (Sigma), 5 μL HALT protease inhibitor (Thermo), and 1 μL Omnicleave (Lucigen). The cell suspension was left for 20 minutes on ice to allow the cells to fully lyse, before the lysates were collected in 1.5 mL microcentrifuge tubes. The lysates were centrifuged at 13,000 RPM for 3 minutes to pellet the debris. The supernatants from the cell lysates were transferred to clean 1.5 mL microcentrifuge tubes. From this point forward, samples were kept on ice.

For Western blotting, 15 μL of cell lysate was combined with 5 μL 4× Novex NuPAGE LDS Sample Buffer supplemented with 1× NuPage Sample Reducing Agent. 15 μL of supernatant culture medium was also prepared for Western blotting in a similar manner. 150 ng of recombinant human ACE2 protein (Abcam, ab151852) was prepared for Western blotting and loaded onto the gel to aid quantification of protein expression, and samples were also prepared from untransfected HEK293 cells as a negative control. The samples were incubated at 80° C. for 5 minutes, then cooled on ice. The entire sample volume was loaded into a Novex WedgeWell 12-well 8-16% tris-glycine mini gel and run for 1-1.5 hour at 165V. A TransBlot Turbo with the PVDF transfer pack (Bio-Rad) was used to transfer the proteins to a PVDF membrane, and the membranes were blocked in 0.2% iBlock (Thermo) with 0.05% Tween-20 in 1×PBS. The membranes were incubated for >1 hour with anti-ACE2 primary antibody (Abcam, ab272690 or ab239924) diluted in blocking buffer. They were then washed twice with 1×TBST (Thermo). The membranes were then incubated for >1 hour with LI-COR donkey anti-rabbit secondary antibody (926-32213) diluted 1:10,000 in blocking buffer. The membranes were then washed four times with 1×TBST, and developed on the LI-COR Odyssey Imaging System.

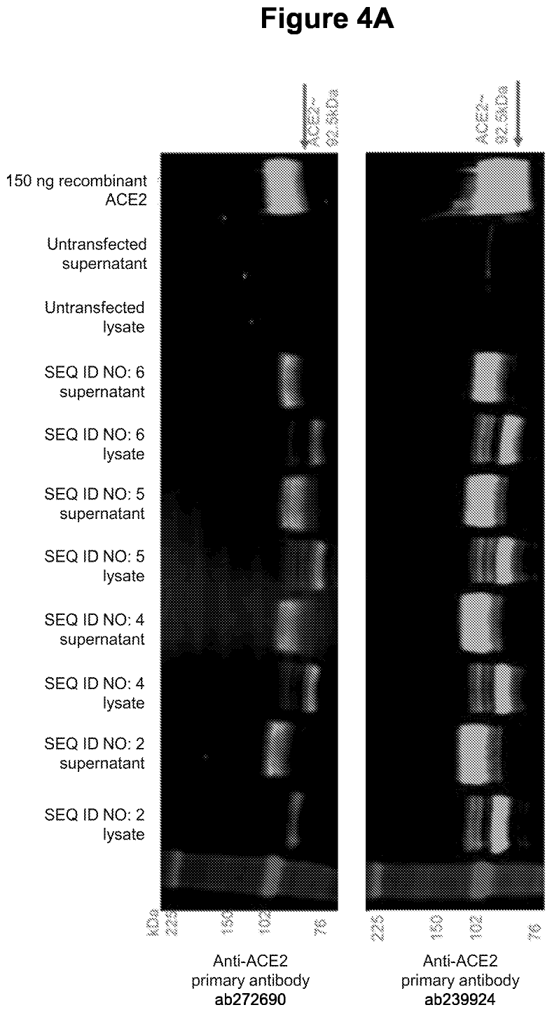

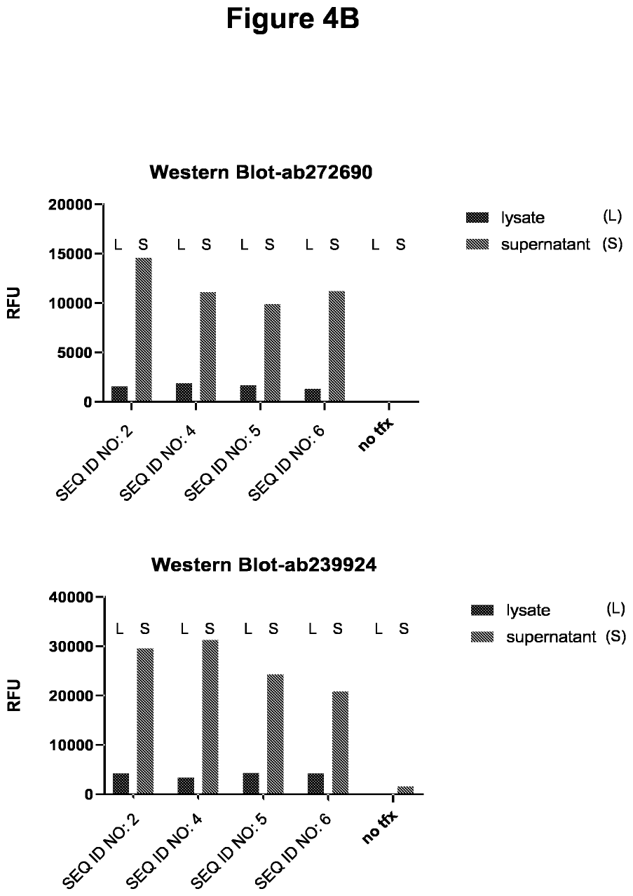

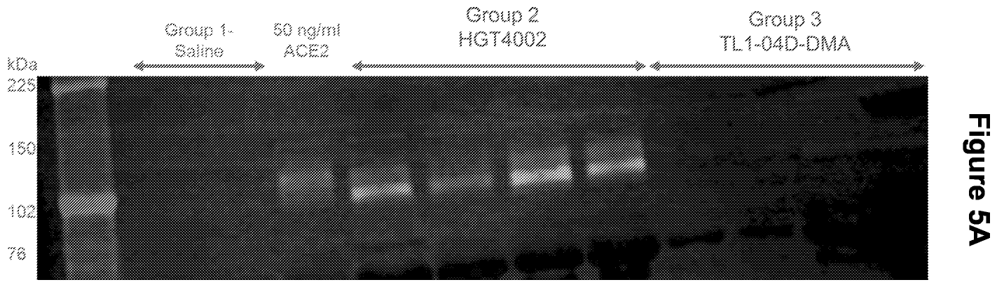

Transfection of mRNAs comprising the optimized nucleotide sequences SEQ ID NO: 2, SEQ ID NO: 4, SEQ ID NO: 5, and SEQ ID NO: 6 described above resulted in high levels of protein expression in HEK293 cell culture. As expected, no ACE2 protein was detected in the samples taken from untransfected HEK293 cells. The two rabbit anti-ACE2 primary antibodies used successfully detected the recombinant ACE2, and sequence-optimized mRNA-encoded polypeptides comprising the extracellular domain of human ACE2 protein, in the cell lysates and supernatant samples. In FIG. 4A, a band of approximately 92.5 kDa is visible in the cell lysate samples for all constructs, which corresponds to the pre-processed extracellular domain of human ACE2 protein. Higher molecular weight bands of approximately 100-110 kDa are visible in the supernatant samples, and to a lesser extent in the lysate samples when using the ab239924 primary antibody, which likely correspond to the maturing and/or mature versions of the sequence-optimized mRNA-encoded polypeptides comprising the extracellular domain of human ACE2 protein which are normally glycosylated multiple times post-translation prior to cellular export. For all mRNAs, the intensity of the bands in the supernatant was notably higher (see e.g. quantification in FIG. 4B), which supports the finding that the polypeptides encoded by the mRNAs comprising optimized nucleotide sequences of the invention are correctly expressed, processed, and secreted as soluble polypeptides into the supernatant. The amino acid substitutions present in the optimized SEQ ID NO: 4, SEQ ID NO: 5, and SEQ ID NO: 6 sequences which increase binding affinity to the SARS-CoV-2 spike (S) glycoprotein did not interfere with expression, processing, or export of the encoded polypeptides in cells.

This example demonstrates that mRNAs comprising an optimized nucleotide sequence encoding a polypeptide comprising the extracellular domain of human angiotensin-converting enzyme 2 (ACE2) protein are expressed, processed and secreted at high levels when transfected into human cells.

### Example 3. Intratracheal Delivery of Different mRNA-LNP Compositions Results in Airway Expression of Soluble Polypeptides Comprising the Extracellular Domain of Human ACE2 Protein

This example illustrates that intratracheal administration of different lipid nanoparticle (LNP) compositions encapsulating an mRNA comprising an optimized nucleotide sequence encoding a polypeptide comprising the extracellular domain of human angiotensin-converting enzyme 2 (ACE2) protein results in expression of the encoded polypeptide by airway epithelial cells and its secretion into the airway lumen in vivo.

mRNAs containing the optimized SEQ ID NO: 2 sequence described in Example 2 were synthesized and purified. The purified mRNA was encapsulated in lipid nanoparticles (LNPs) comprising a cationic lipid, a non-cationic lipid, a cholesterol-based lipid, and a PEG-modified lipid. Compositions comprising the LNPs were prepared as aqueous suspensions. Different LNP compositions were tested—one formulation used HGT4002 as the cationic lipid, and another formulation used TL1-04D-DMA as the cationic lipid.

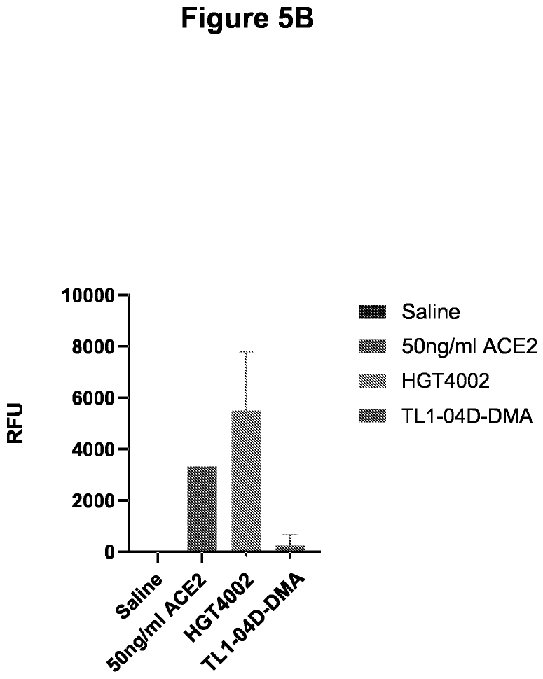

Two groups of male CD-1 mice were administered a 15 μg mRNA dose of the mRNA-LNP compositions by intratracheal instillation, and bronchoalveolar lavage fluid (BALF) samples were taken 24 hours after LNP administration. Group 2 mice received LNPs containing HGT4002, whereas group 3 mice received LNPs containing TL1-04D-DMA. As a negative control, saline solution without LNPs was administered to a control group of mice (Group 1). BALF samples were taken from 4 animals in each group treated as indicated. The BALF samples from the mice were processed and analysed by Western blotting using anti-ACE2 primary antibodies as described in Example 2 above, and the results are shown in FIG. 5A and FIG. 5B. A sample of recombinant human ACE2 protein was also included in the experiment as a positive control.

Intratracheal delivery of both LNP compositions resulted in detectable expression of the sequence-optimized mRNA-encoded polypeptides comprising the extracellular domain of human ACE2 protein in BALF of group 2 and group 3 mice. BALF levels of the encoded polypeptide in samples from group 2 mice were comparable to the positive control (approx. 50 ng/mL, see FIG. 5B). No sequence-optimized mRNA-encoded polypeptides comprising the extracellular domain of human ACE2 protein was detected in the serum of any of the animals, indicating that intratracheal administration of LNPs led to local expression in the airway epithelial cells.

Taken together, the data in this example demonstrate that the delivery of different LNP compositions encapsulating the mRNAs of the invention results in expression and secretion of the encoded polypeptides in the airways of test animals. As the sequence-optimized mRNA-encoded polypeptides comprising the extracellular domain of human ACE2 protein are present in soluble form in the airways, they are available to serve as ‘decoy receptors’ by binding to and neutralizing viruses which use the ACE2 receptor for cellular entry (e.g. SARS-CoV-2). Expression of the encoded polypeptides was localized to the airways, and no ‘leakage’ of the ACE2 ‘decoy receptor’ polypeptides was detected in the systemic circulation. The data also indicate that certain LNP compositions may be more effective than others in inducing expression of the mRNA-encoded polypeptide in the lung.

### Example 4. Variant Polypeptides Comprising the Extracellular Domain of Human ACE2 Protein are Expressed from Sequence-Optimized mRNAs Following Intratracheal Delivery

This example illustrates that amino acid substitutions designed to increase binding affinity to the SARS-CoV-2 spike (S) glycoprotein do not interfere with expression and secretion of variant polypeptides comprising the extracellular portion of human ACE2 protein. Intratracheal administration of LNPs encapsulating sequence-optimized mRNA encoding these variant polypeptides were detected in the airways of test animals at levels comparable to those achieved with mRNAs encoding corresponding polypeptides without these amino acid substitutions.

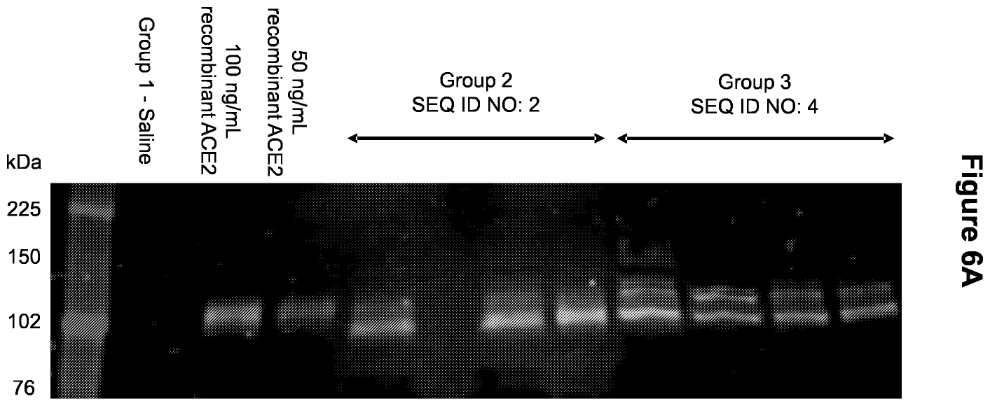

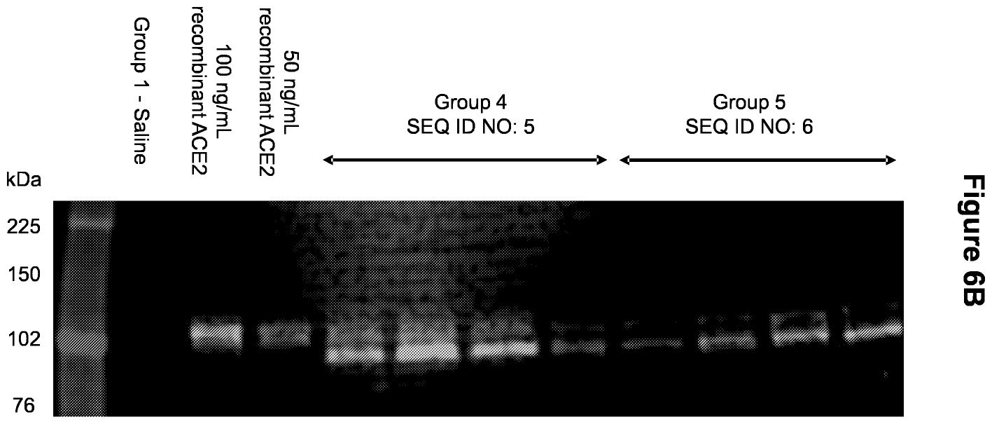

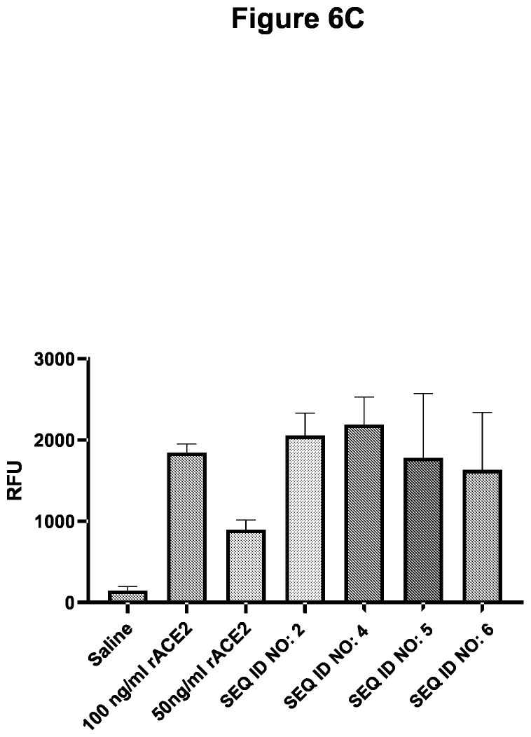

mRNAs comprising the optimized nucleotide sequences SEQ ID NO: 2, SEQ ID NO: 4, SEQ ID NO: 5, and SEQ ID NO: 6 described in Example 2 were synthesized, purified, and encapsulated in LNPs as described in Example 3, using HGT4002 as the cationic lipid. Four groups of male CD-1 mice were administered a 15 g mRNA dose of the LNP compositions by intratracheal instillation, and BALF samples were taken 24 hours after LNP administration. Group 2, group 3, group 4 and group 5 mice received mRNA with SEQ ID NO: 2, SEQ ID NO: 4, SEQ ID NO: 5, and SEQ ID NO: 6 as the coding sequence, respectively. As a negative control, saline solution without LNPs was administered to a control group of mice (Group 1). BALF samples were taken from 4 animals in each group, treated as indicated, to determine the level of expression of the secreted polypeptides. The BALF samples from the mice were processed and analysed by Western blotting as described in Examples 3 and 4 above, and the results are shown in FIG. 6A and FIG. 6B. Samples of recombinant human ACE2 protein at 100 ng/mL and 50 ng/mL were also included in the experiment as positive controls for detection, and to aid quantification. Quantification of the western blot fluorescence signals is shown in FIG. 6C.

The western blot data in FIG. 6A show that polypeptides comprising the extracellular domain of human ACE2 protein encoded by both SEQ ID NO: 2 and SEQ ID NO: 4 were detected in BALF samples. The polypeptides encoded by SEQ ID NO: 2 and SEQ ID NO: 4, respectively, displayed similar expression levels, estimated to be approximately 100 ng/mL. Similarly, polypeptides comprising the extracellular domain of human ACE2 protein encoded by SEQ ID NO: 5 and SEQ ID NO: 6 were detected in in BALF samples, at a concentration of approximately 100 ng/mL in most cases, as shown in FIG. 6B. In addition to the main band of approximately 92.5 kDa corresponding to the pre-processed extracellular domain of human ACE2 protein visible in the samples for all optimized nucleotide sequences, higher molecular weight bands of approximately 100-110 kDa were visible in the samples, indicating that the more mature and/or glycosylated form of the encoded polypeptides were also expressed and secreted into the airway lumen. The quantification of western blot signals in FIG. 6C indicates that the extracellular domain of human ACE2 protein encoded by SEQ ID NO: 2, and the variants encoded by SEQ ID NO: 4, SEQ ID NO: 5, and SEQ ID NO: 6, show similar levels of protein expression in BALF samples.

The findings in this example confirm that amino acid substitutions designed to increase binding affinity to the SARS-CoV-2 spike (S) glycoprotein do not interfere with expression and secretion of the variant polypeptides into the airway lumen, suggesting that they are available to neutralize viral particles in the airway lumen and thus may be able to prevent infection with viruses that require the human ACE2 protein to gain entry into cells.

### Example 5. Prolonged Airway Detection of Polypeptides Comprising the Extracellular Domain of Human ACE2 Protein Following Intratracheal Administration of mRNA-LNPs

This example demonstrates that the polypeptides comprising the extracellular domain of human ACE2 protein encoded by the mRNAs of the invention are detectable in the airways of test animals for up to 8 days following intratracheal administration of LNP compositions encapsulating the mRNAs (mRNA-LNPs).

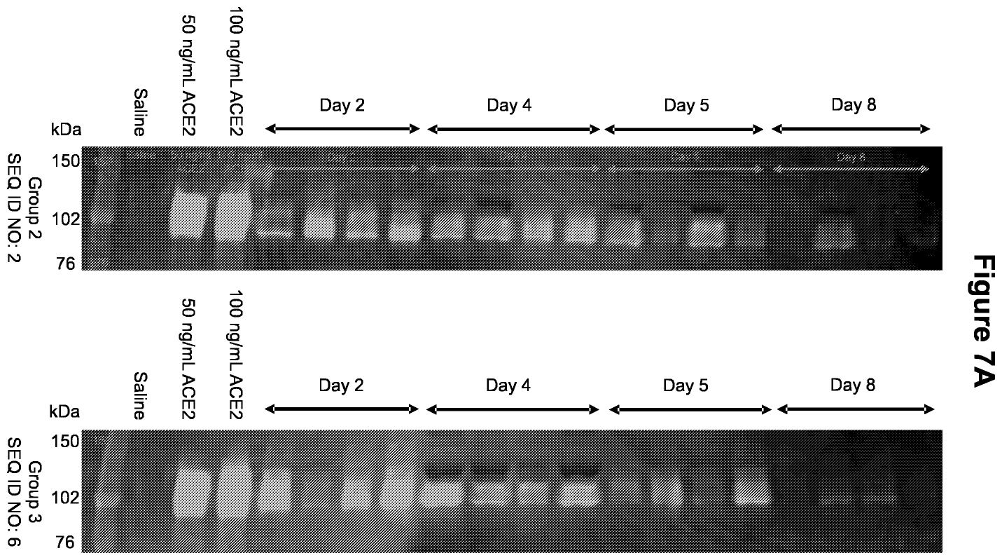

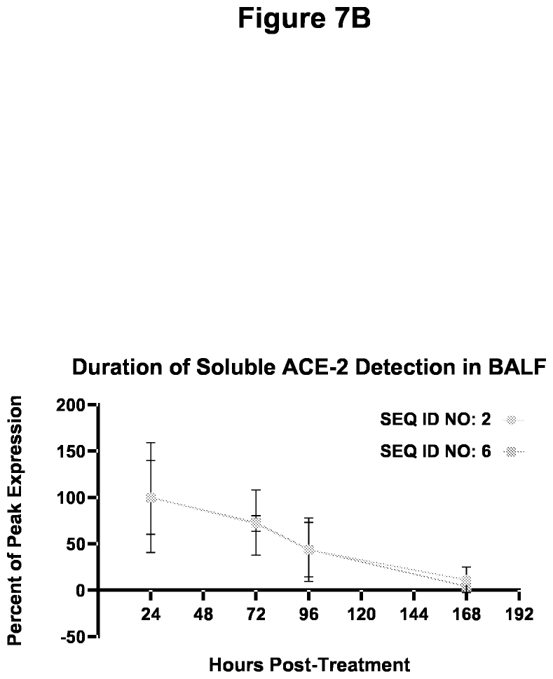

mRNAs comprising the optimized nucleotide sequences SEQ ID NO: 2 and SEQ ID NO: 6 described in Example 2 were synthesized, purified, and encapsulated in LNPs as described in Example 3, using HGT4002 as the cationic lipid. Two groups of male CD-1 mice were administered a 15 μg mRNA dose of the LNP compositions by intratracheal instillation. Group 2 received mRNA with SEQ ID NO: 2 as the coding sequence, group 3 received mRNA with SEQ ID NO: 6 as the coding sequence. As a negative control, saline solution without LNPs was administered to a control group of mice (group 1). BALF samples were taken from 4 animals in each group treated as indicated to determine the level of expression of the secreted polypeptides. The BALF samples from the mice were processed and analysed by western blotting as described in Examples 3 and 4 above, at the following time points after LNP administration: 24 hours (Day 2), 72 hours (Day 4), 96 hours (Day 5), and 168 hours (Day 8). The results are shown in FIG. 7A. Samples of recombinant human ACE2 protein at 100 ng/mL and 50 ng/mL were also included in the experiment as positive controls for detection, and to aid quantification. Quantification of the western blot fluorescence signals is shown in FIG. 7B.

The data in this example show that the sequence-optimized mRNA-encoded polypeptides comprising the extracellular domain of human ACE2 protein could be detected in BALF samples in animals treated with the LNP compositions up to 8 days after intratracheal administration. As shown in FIG. 7B, the levels of the polypeptides detected in the BALF samples decreased in a linear fashion at the measured time points throughout the experiment.

The sustained duration of detectable expression of sequence-optimized mRNA-encoded polypeptides comprising the extracellular domain of human ACE2 protein up to 8 days following administration is advantageous in conferring protection against infection by viruses which use the human ACE2 receptor for cellular entry. For example, a single administration of the mRNA-LNPs of the invention may confer protection against infection by SARS-CoV-2 for several days (e.g. 3 to 4 days post administration). Therefore a single administration of the mRNA-LNPs may be effective as post-exposure prophylaxis. For example, a single administration of the mRNA-LNPs may provide protection during the crucial incubation period of COVID-19 (before onset of symptoms) which lasts 5-6 days, on average. Alternatively, it may also be effective in an acute disease setting where a rapid reduction in viral titres in the lung may allow for a subject to recover and mount an effective immune response on their own, e.g. in forming of virus-neutralizing antibodies.

### Example 6. Expression of mRNA-Encoded Polypeptides Comprising the Extracellular Domain of Human ACE2 Proteins Fused to an Immunoglobulin Fc Region

This example demonstrates that sequence-optimized mRNAs encoding a polypeptide comprising the extracellular domain of ACE2 protein fused to an immunoglobulin Fc region result in high levels of protein expression.

Various exemplary optimized nucleotide sequences encoding the extracellular domain of human ACE2 protein fused to the immunoglobulin Fc region from human IgG1 were designed, as schematically illustrated in FIG. 4.

SEQ ID NO: 3, SEQ ID NO: 7, SEQ ID NO: 8, and SEQ ID NO: 9 encode polypeptides in which the extracellular domain of the human ACE2 protein and the immunoglobulin Fc region are linked by a ‘GS linker’ (SEQ ID NO: 28) to separate the two domains and provide flexibility to allow independent folding of each domain during protein expression. The polypeptide encoded by the SEQ ID NO: 7 nucleotide sequence additionally comprises a ‘KLKL’ localization sequence (SEQ ID NO: 29), linked to the C-terminus of the immunoglobulin Fc region by a further GS linker. The polypeptide encoded by the SEQ ID NO: 8 nucleotide sequence additionally comprises a ‘RLRL’ localization sequence (SEQ ID NO: 30), linked to the C-terminus of the immunoglobulin Fc region by a further GS linker. The polypeptide encoded by the SEQ ID NO: 9 nucleotide sequence additionally comprises a human immunoglobulin J chain (SEQ ID NO: 31), linked to the C-terminus of the immunoglobulin Fc region by a further GS linker. The KLKL localization sequence, RLRL localization sequence, and J chain bind to the polymeric Ig receptor (pIgR) which are expressed on the surface of lung epithelial cells. They can facilitate transcytosis across the airway epithelium, such that polypeptides including one of these sequences may be more effectively delivered to the lung lumen (Borrok et al., JCI Insight 2018 Jun. 21; 3(12): e97844).

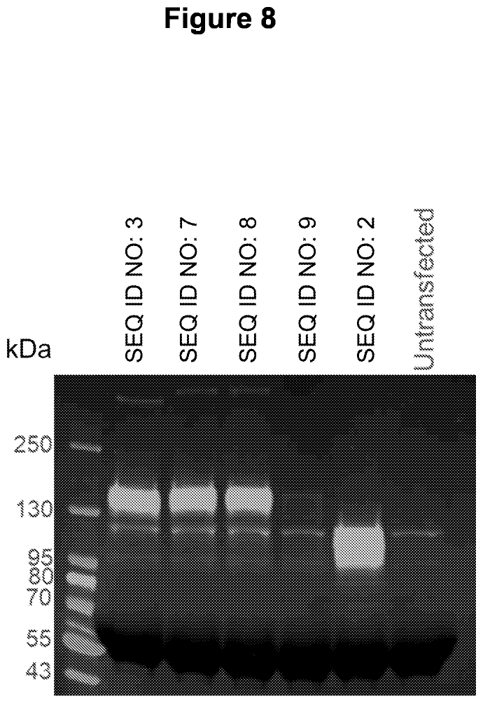

mRNAs with SEQ ID NO: 2, SEQ ID NO: 3, SEQ ID NO: 7, SEQ ID NO: 8, and SEQ ID NO: 9 as the coding sequence, respectively, were synthesized and used to transfect HEK293 cells in culture as described in Example 2. Transfected cells were incubated and samples processed for Western blotting, also as described in that example. The results of the western blot are depicted in FIG. 8.

Apart from the polypeptide encoded by the SEQ ID NO: 9 construct, all of the polypeptides comprising the extracellular domain of human ACE2 protein and an immunoglobulin Fc region, as described above, displayed high levels of protein expression in HEK293 cells. The fused immunoglobulin Fc regions, KLKL localization sequence, and RLRL localization sequence did not interfere with protein expression. Presence of a faint band of approximately 150 kDa for the SEQ ID NO: 9 construct suggests that fusion proteins containing the J chain are not appropriately expressed in HEK293 cells, perhaps due to protein aggregation or instability.

Taken together, the data in this example demonstrate that mRNAs comprising optimized nucleotide sequences encoding a polypeptide comprising the extracellular domain of human ACE2 protein and an immunoglobulin Fc region can be expressed at high levels.

### Example 7. Effective Cross-Epithelial Translocation of Polypeptides Comprising the Extracellular Domain of Human ACE2 Protein and an Immunoglobulin Fc Region

This example demonstrates that sequence-optimized mRNA-encoded polypeptides comprising the extracellular domain of human ACE2 protein and an immunoglobulin Fc region effectively translocate across a differentiated epithelial cell layer.

This example explores whether the secreted Fc fusion polypeptides comprising the extracellular domain of human ACE2 protein are able to effectively translocate from the blood circulation across the airway epithelial layer into the lumen of the lungs. If translocation occurs efficiently, mRNAs encoding ACE2 ‘decoy receptors’ may also be delivered by e.g. intravenous administration. This may be advantageous, for example, in an acute cases of COVID-19 infection, where therapy via localized delivery into the lungs may not or no longer be a feasible treatment modality in order to neutralize virus particles in the lung.

To study the translocation capability of Fc fusion polypeptides described in Example 6, a differentiated epithelial cell layer composed of human bronchial epithelial cells (HBECs) was prepared to simulate the air-liquid interface (ALI) of the lung epithelium. The HBEC-ALI technique is advantageous as it reproduces a well differentiated airway epithelium with distinct, functional cells, allowing it to be used as a highly translatable airway cell model.

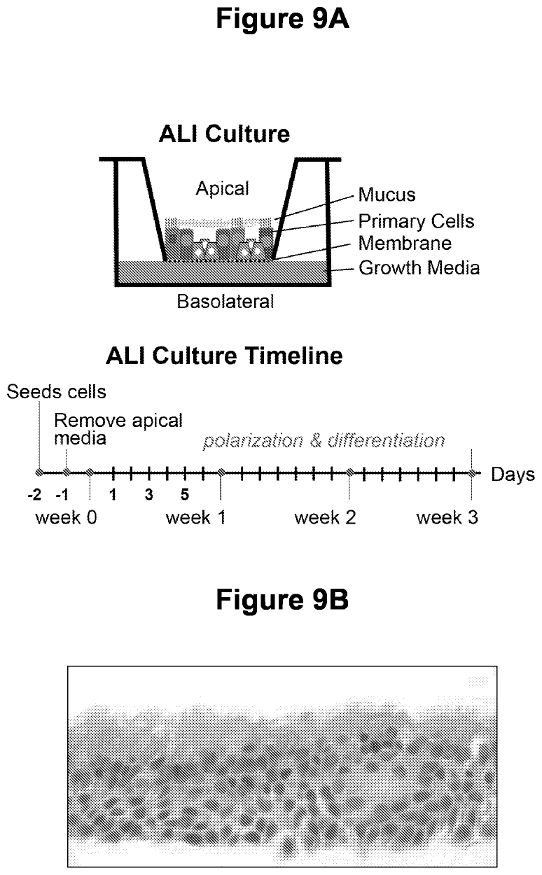

To establish an HBEC-ALI culture, human bronchial epithelial cells were seeded in transwell plates and grown submerged in culture medium for 7 days. Upon reaching confluency, the medium on the apical surface of the epithelial cell layer was removed, and only basal growth culture medium was maintained. Cells were cultured in ALI format for 28 days to allow polarization and differentiation before experiments were performed, as shown in FIG. 9A. The exemplary HBEC-ALI system schematic is shown in FIG. 9A. The differentiated epithelium were sectioned and stained with hematoxylin and eosin (H&E), as shown in FIG. 9B, which indicates the presence of multi-ciliated cells that can be used as airway cell model.

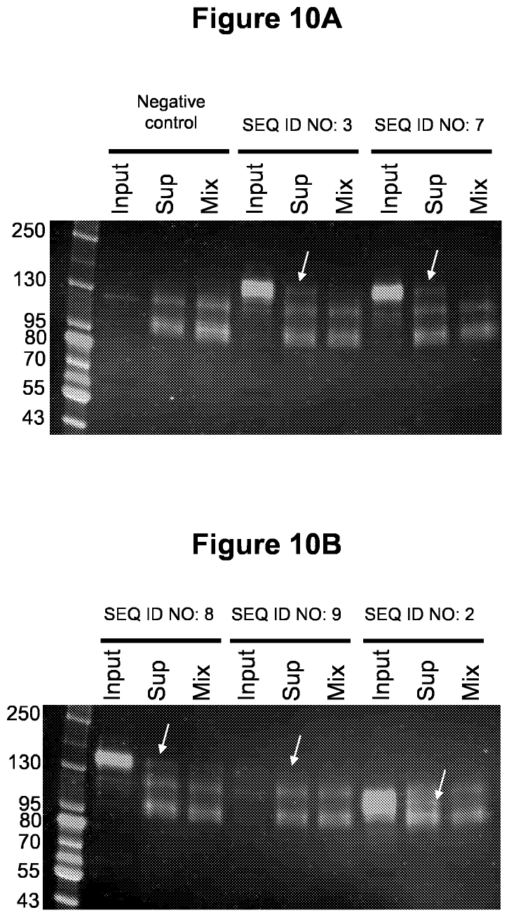

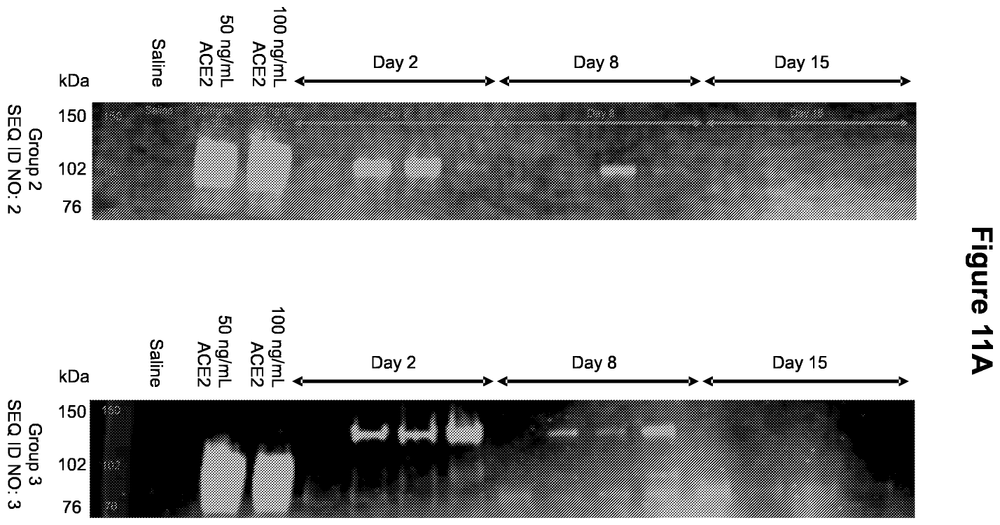

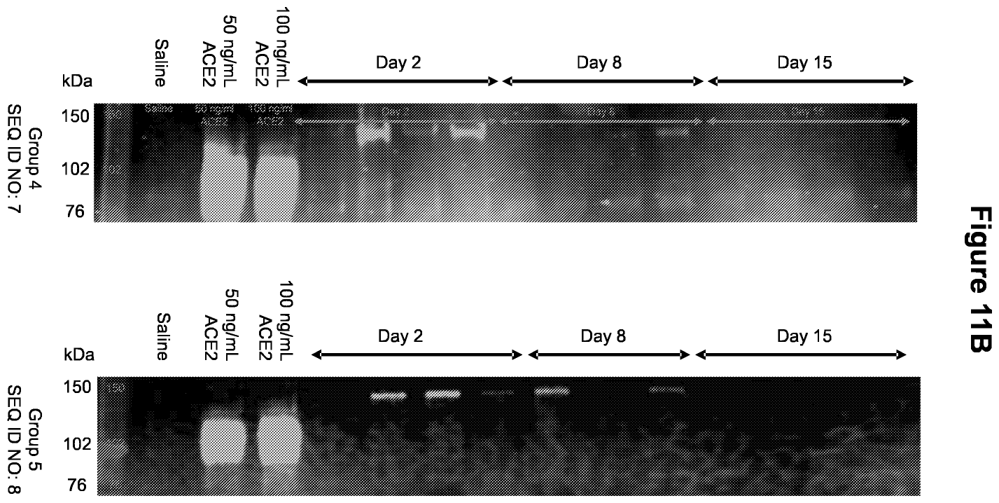

Briefly, HEK293 cells in culture were grown and transfected with the sequence-optimized mRNA constructs described in Example 6 (comprising SEQ ID NO: 2, SEQ ID NO: 3, SEQ ID NO: 7, SEQ ID NO: 8, and SEQ ID NO: 9, respectively). The optimized mRNA comprising SEQ ID NO: 2, encoding a polypeptide comprising the extracellular domain of human ACE2 without an immunoglobulin Fc region or localization sequence, was included as a control. The ACE2 polypeptides were expressed by the HEK293 cells and secreted into the cell culture medium. The conditioned medium was removed, and used to replace the medium in contact with the basolateral side of the HBEC cells in the previously established ALI cultures (labelled as ‘Sup’ in FIG. 10). As HBEC ALI cultures can be sensitive to changes in the medium composition, the conditioned medium was mixed in some instances with the HBEC ALI culture medium in a 1:1 ratio (labelled as ‘Mix’ in FIG. 10). 48 hours after media replacement, the apical side of the HBEC ALI cultures was washed with PBS to resuspend the ACE2 polypeptides which had translocated from the basolateral surface of the HBEC cells to the mucus layer at the apical surface (‘mucus wash’). The mucus wash samples were prepared analysed by Western blotting using an anti-ACE2 primary antibody as described in Examples 2 and 3 above. As a negative control, mucus wash samples were taken from HBEC ALI cultures treated with medium from untransfected HEK293 cells. 1 μL of culture media from the transfected or untransfected HEK293 cells were also included as positive and negative controls.

The results of this experiment are shown in FIGS. 10A and 10B. Multiple bands were observed in lanes corresponding to the mucus wash samples, which may indicate cross-reactivity of the primary or secondary antibodies to proteins present in these samples. The band corresponding to each Fc fusion polypeptide can be compared to the band in the matched ‘input’ sample obtained from HEK293 cells, and these bands are marked with white arrows in FIG. 10A and FIG. 10B.

Successful translocation of the secreted polypeptide across the HBEC epithelial cell layer was observed for all of the tested mRNAs. Surprisingly, the control polypeptide comprising the extracellular domain of ACE2 protein alone (without a fused immunoglobulin Fc region or localization sequence, SEQ ID NO: 2) was also detected in the HBEC mucus wash sample, indicating that the Fc region and/or localization sequences may not be strictly required for successful translocation. The level of the polypeptide containing a J chain, encoded by SEQ ID NO: 9, was much lower than the level of the other polypeptides, consistent with its poor expression in HEK293 cells.

Taken together, these data indicate that sequence-optimized mRNA-encoded polypeptides comprising the extracellular domain of human ACE2 protein and an immunoglobulin Fc region effectively translocate across the epithelial cell layer in HBEC-ALI cultures. Such polypeptides are therefore expected to effectively translocate across airway epithelial cells to neutralize viral particles in the airway lumen (including the lungs), thereby conferring protection against viruses which use the human ACE2 receptor for cellular entry.

### Example 8. Prolonged Detection of mRNA-Encoded Human ACE2 Polypeptides Encoded Following Intravenous Administration of mRNA-LNP Compositions

This example demonstrates that LNP compositions encapsulating sequence-optimized mRNAs encoding polypeptides comprising the extracellular domain of human ACE2 protein and an immunoglobulin Fc region are expressed in vivo and effectively translocated to the airway lumen following intravenous administration of the LNP compositions. The mRNA-encoded polypeptides remained detectable in the airway and serum of test animals for up to 8 days post administration.

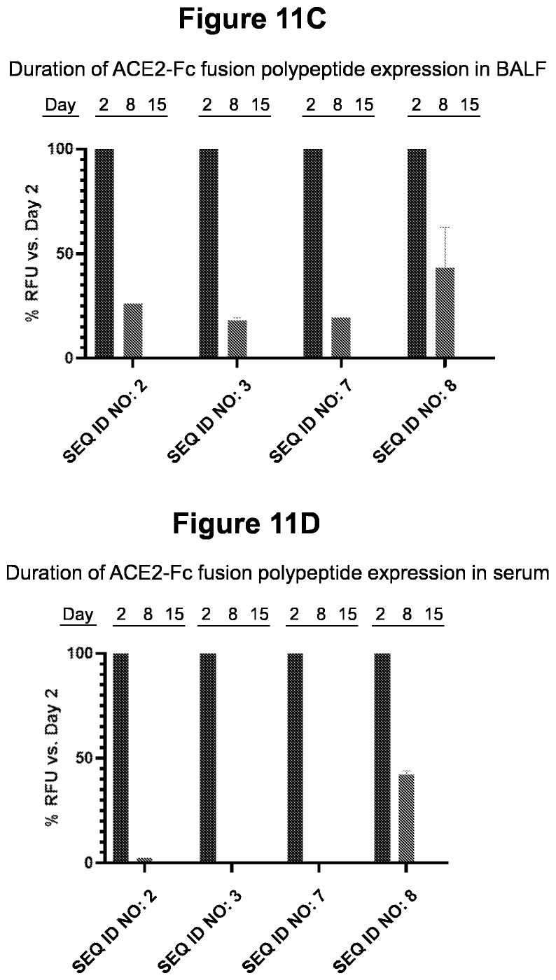

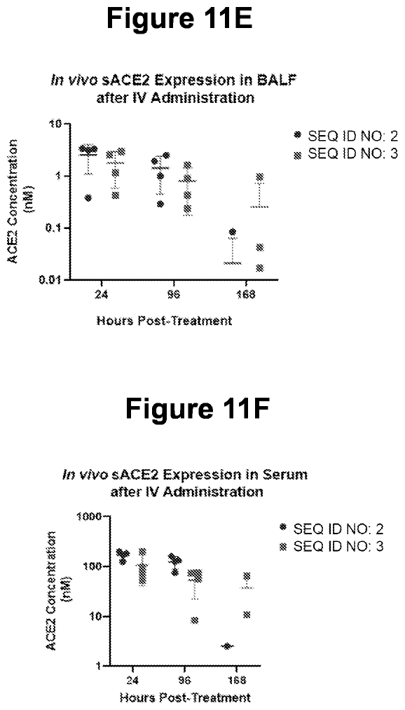

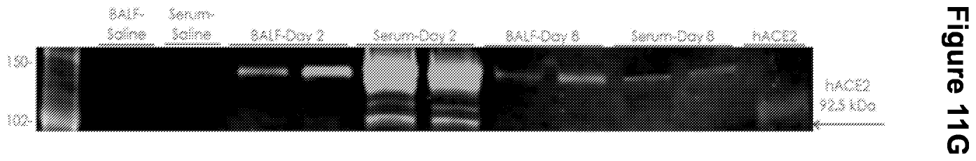

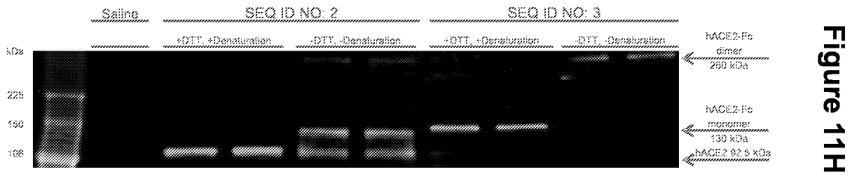

mRNAs comprising SEQ ID NO: 2, SEQ ID NO: 3, SEQ ID NO: 7, and SEQ ID NO: 8 as the coding sequence, respectively, were synthesized, purified, and encapsulated in LNPs as described in Example 5, using ML-2 as the cationic lipid. LNP compositions encapsulating the mRNA were prepared as aqueous suspensions for intravenous delivery. Groups of male CD-1 mice were administered a dose of the LNP formulations at a dose level of 1 mg/kg by tail vein injection. As a negative control, saline solution without LNPs was administered to a control group of mice. BALF samples were taken from 4 animals in each group treated as indicated to determine the level of secreted ACE2 polypeptides which successfully translocated to the airway lumen of the lungs. The BALF samples from the mice were processed and analysed by western blotting using an anti-ACE2 primary antibody as described in Examples 2 and 3 above at the following time points after LNP administration: 24 hours (Day 2), 72 hours (Day 4), 96 hours (Day 5), and 168 hours (Day 8). Samples from the serum of the mice were also taken at these time points to measure the amount of secreted ACE2 polypeptides present in the systemic circulation. The western blot results from BALF samples are shown in FIG. 11A and FIG. 111B. Samples of recombinant human ACE2 protein at 100 ng/mL and 50 ng/mL were also included in the experiment as positive controls for detection, and to aid quantification. Quantification of the western blot fluorescence signals from BALF samples is shown in FIG. 11C, and quantification of the western blot fluorescence signals from the serum samples is shown in FIG. 11D.

The sequence-optimized mRNA-encoded polypeptides comprising the extracellular domain of human ACE2 protein and an immunoglobulin Fc region were detected in mouse BALF samples up to 8 days after administration of the LNP compositions. At Day 8, there was a higher remaining level of detectable ACE2-Fc fusion protein in the BALF samples, compared to the serum samples (FIG. 11D). The polypeptide encoded by SEQ ID NO: 8 displayed higher detectable expression than the polypeptides encoded by the other sequences at Day 8 in both BALF and serum samples, indicating that it may be more stable in vivo.

An additional study was performed to evaluate intravenous (IV) administration of sACE2 in vivo. Mice were treated with a single IV dose of either sACE2 (e.g., encoded by SEQ ID NO: 2) or sACE2-Fc (e.g., encoded by SEQ ID NO: 3) mRNA LNPs and ACE2 western blots were performed on both the serum and BALF samples isolated from treated mice over a one-week period. High levels of sACE2 and sACE2-Fc proteins were detected in serum at 24 hr post dose. Both sACE2 and sACE2-Fc protein were detected in the BALF of treated animals out to 7 days after IV dosing (FIG. 11E), though as expected, the observed protein levels in BALF were much lower than the observed expression in serum (FIG. 11E). FIG. 11G shows an exemplary Western blot comparing expression of soluble ACE2 polypeptide encoded by SEQ ID NO: 3 in BALF fluid and serum after intravenous dosing. These data support the notion that expressing high levels (50-200 nM) of sACE2 in circulation affords the ability of the protein to translocate from the bloodstream into the lumen of the lung, potentially giving the option for a second route of administration for sACE2 therapy.

Western blots of reduced and unreduced samples using non-Fc fusion sACE2 and sACE2-Fc protein present in mouse serum show a higher molecular weight band in non-reduced sACE2-Fc samples that was absent from the non-Fc fusion and reduced controls (FIG. 11H). 20 μg of sample were subjected to reducing and denaturing conditions (DDT and incubation at 80° C.) or left untreated prior to analysis. This corresponds to the predicted molecular weight of a sACE2-Fc dimer and confirms that mRNA-derived sACE2-Fc fusion proteins are capable of dimerization in vivo.

The sustained duration of detectable expression of mRNA-derived polypeptides comprising the extracellular domain of human ACE2 protein and an immunoglobulin Fc region in the airway lumen and in the general circulation, up to 8 days following administration, is advantageous in conferring protection against infection by viruses which use the human ACE2 receptor for cellular entry. For example, a single administration of LNPs comprising the mRNAs of the invention may confer significant protection against infection by SARS-CoV-2 for several days (e.g. up to 8 days for the polypeptide encoded by SEQ ID NO: 8) in the lung. Sustained levels of the mRNA-expressed polypeptides in serum may also provide effective protection against systemic viral infection by neutralizing the viral particles present in circulation and preventing or reducing infection of distal organs and tissues accessible via the systemic circulation, thereby providing a broader therapeutic benefit than achievable by localized administration of the mRNAs of the invention.

In summary, this example demonstrates that sequence-optimized mRNA-encoded polypeptides comprising the extracellular domain of human ACE2 protein and an immunoglobulin Fc region are expressed in vivo, effectively secreted into the serum and translocate to the airway lumen of the lungs, where they remain detectable for up to 8 days following intravenous administration. These observations confirm the in vitro findings summarized in Example 7.

### Example 9. Prolonged Detection of an mRNA-Encoded Polypeptide Comprising the Extracellular Domain of Human ACE2 Protein Fused to an Immunoglobulin Fc Region Following Intratracheal Administration

This example demonstrates that a polypeptide comprising the extracellular domain of human ACE2 protein fused to an immunoglobulin Fc region (sACE2-Fc) encoded by an mRNA of the invention is expressed in the airways of both CD-1 mice and Syrian Golden hamsters following intratracheal administration of LNP compositions encapsulating the mRNA (mRNA-LNP).

mRNAs comprising SEQ ID NO: 3 as the coding sequence, were synthesized, purified, and encapsulated in LNPs as described in Example 5, but using SY-3-E14-DMAPr as the cationic lipid. The LNPs comprised SY-3-E14-DMAPr, DOPE, cholesterol and DMG-PEG2K at molar ratios of 50:30:15:5. The N/P ratio was about 4. This LNP composition encapsulating the mRNA was prepared for intratracheal delivery. Groups of male CD-1 mice and Syrian Golden hamsters were administered a dose 15 μg and 30 μg mRNA, respectively, of the LNP composition by intratracheal instillation. To assess peak expression, mice and hamsters in this example were sacrificed 24 hours after intratracheal delivery of the LNPs.

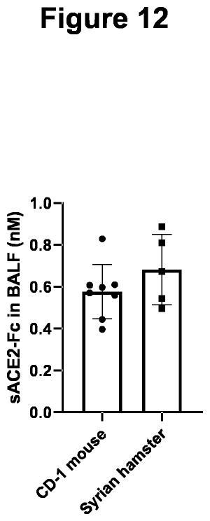

The data in FIG. 12 show that the mRNA-encoded sACE-Fc was detected in the BALF of mice and hamsters sacrificed 24 hours following intratracheal delivery. These data demonstrate that sACE2-Fc is highly expressed in different in vivo models after dosing and confirm the findings of Example 5 with a corresponding Fc fusion protein. Moreover, this example provides animal model data demonstrating that a single administration of the mRNA-LNPs of the invention may confer protection against infection by SARS-CoV-2, and may be effective as post-exposure prophylaxis. It may also be effective in an acute disease setting. Furthermore, the data demonstrate that findings in mice can be extrapolated to a larger rodent model that is more suitable for virus challenge studies.

### Example 10. Effective Neutralization of S Glycoprotein-Displaying Pseudoviruses with Polypeptides Comprising the Extracellular Domain of Human ACE2 Protein

This example demonstrates that sequence-optimized RNA-encoded polypeptides comprising the extracellular domain of human ACE2 protein can effectively neutralize pseudotyped reporter virus particles (RVPs) displaying the SARS-CoV-2 spike (S) glycoprotein in a cell culture model of infection. It also demonstrates that the amino acid substitutions in said polypeptides designed to increase binding affinity to the SARS-CoV-2 spike (S) glycoprotein increase the efficacy of RVP neutralization. Furthermore, the data in this example confirm that the sequence-optimized RNA-encoded polypeptides comprising the extracellular domain of human ACE2 protein can effectively neutralize RVPs displaying different spike (S) glycoprotein variants from several widespread variants of SARS-CoV-2.

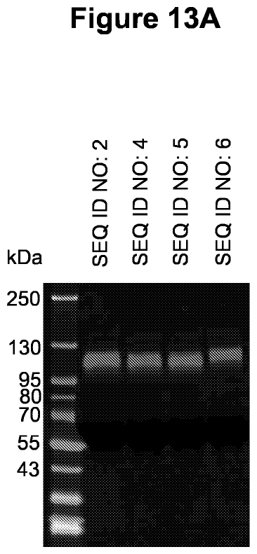

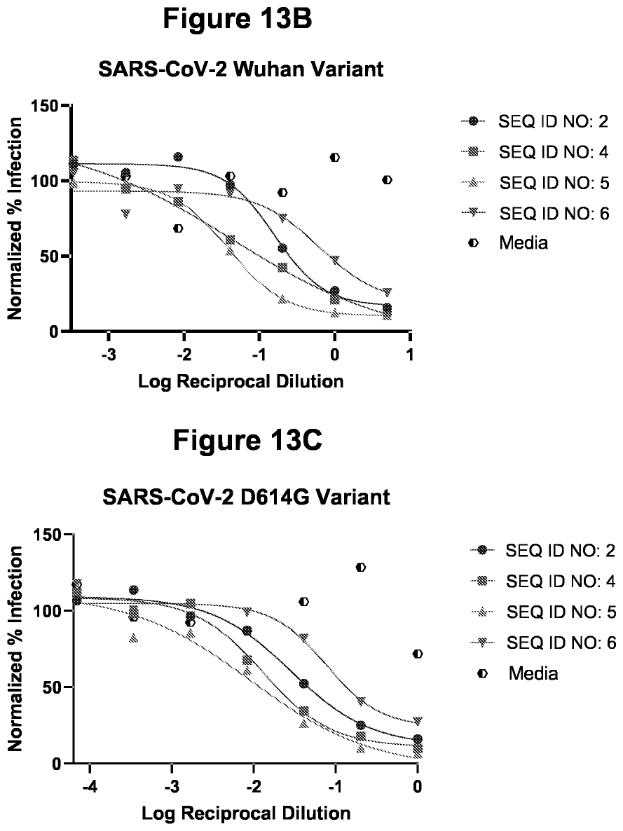

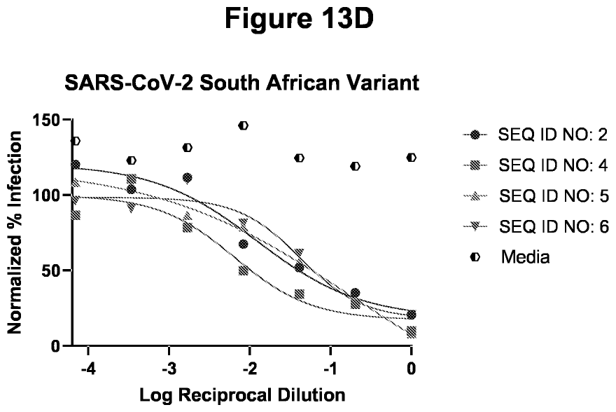

HEK293 cells expressing the human ACE2 receptor (hACE2-293 cells) were generated as target cells for infection with RVPs. RVPs can be rapidly engineered to express existing SARS-CoV-2 variant spike proteins, as well as newly emerging variant spike proteins, to allow for testing of neutralization potential against a wide variety of variants of interest or concern. Different RVPs displaying variants of the SARS-CoV-2 spike (S) glycoprotein derived from notable widespread SARS-CoV-2 variants (Wuhan, D614G, B.1.1.7 (‘UK variant’), and 20H/501Y.V2 (‘South African variant’) were generated. The RVPs contain a luciferase reporter gene which is expressed in hACE2-293 cells after successful infection. mRNAs comprising optimized nucleotide sequences encoding the extracellular domain of human ACE2 protein as described in Example 2 (SEQ ID NO: 2, SEQ ID NO: 4, SEQ ID NO: 5, and SEQ ID NO: 6) were transfected into HEK293 cells (which were not transformed to express the human ACE2 receptor). Samples of the conditioned culture medium of the transfected cells were processed for western blotting with an anti-ACE2 primary antibody as described in Example 2 above to confirm successful expression and secretion of the mRNA-encoded polypeptides. The results are shown in FIG. 13A.

Serial dilutions of the SARS-CoV-2 RVPs were prepared, and mixed for 1 hour at 37° C. with the different conditioned HEK293 cell culture media. 96-well plates of 50% confluent 293T-hACE2 clonal cells in 75 μL volume were inoculated with 50 μL of the mixtures of SARS-CoV-2 RVPs and HEK293 media containing secreted polypeptides comprising the extracellular domain of human ACE2 protein, and incubated at 37° C. for 72 h. The 293T-hACE2 cells stably express the ACE2 receptor which can be used by the SARS-CoV-2 RVPs for cellular entry and infection. Luciferase activity was then assayed using the Promega Dual-Glo® Luciferase Assay system, and plates were scanned and luciferase activity quantified using an appropriate imaging system. Infection of 293T-hACE2 cells by SARS-CoV-2 RVPs were measured via luciferase signal. The measured luciferase signal was expressed as ‘normalized % infection’, indicating the normalized percentage of 293T-hACE2 cells infected as a percentage of the average luciferase signal measured for control 293T-hACE2 cells incubated with medium which did not contain secreted polypeptides comprising the extracellular domain of human ACE2 protein. A decrease in luciferase signal following incubation of the RVPs with the conditioned HEK293 cell culture medium indicates neutralization of the RVPs. The results of the neutralization assay are shown in panels C-D of FIG. 13.

The polypeptides comprising the extracellular domain of human ACE2 protein encoded by the optimized nucleotide sequence of SEQ ID NOs: 2, 4, 5, and 6, respectively, were able to neutralize RVPs displaying the spike (S) glycoprotein of the SARS-CoV-2 Wuhan reference strain and the SARS-CoV-2 D614G variant which first spread quickly worldwide during the COVID-19 pandemic (FIG. 13B). The polypeptides were also able to neutralize RVPs displaying the spike (S) glycoprotein of the SARS-CoV-2 variants B.1.1.7 (‘UK variant’; see FIG. 13C), and 20H/501Y.V2 (‘South African variant’; see FIG. 13D). The polypeptides encoded by SEQ ID NO: 4 and SEQ ID NO: 5 appear similarly or more capable of neutralizing RVPs compared to the polypeptides encoded by SEQ ID NO: 2 and SEQ ID NO: 6, at the same dilutions (FIGS. 13B and 13C).

Taken together, the data in this example demonstrate that sequence-optimized RNA-encoded polypeptides comprising the extracellular domain of human ACE2 protein can effectively neutralize pseudotyped reporter virus particles (RVPs) displaying various variant SARS-CoV-2 spike (S) glycoproteins in a cell culture model of infection. The data therefore provide experimental support for the neutralizing potency of such mRNA-encoded polypeptides in vivo to treat or prevent infections caused by viral pathogens that bind to the human ACE2 protein to enter a cell. In particular, they support the feasibility of a variant agnostic treatment for, or prevention of, SARS-CoV-2 infection using mRNA therapy. Moreover, the data indicate that an extracellular domain of human ACE2 protein comprising amino acid substitutions designed to increase its binding affinity to the SARS-CoV-2 spike (S) glycoprotein may be more effective in neutralizing the SARS-CoV-2 virus.

### Example 11. Effective Neutralization of S Glycoprotein-Displaying Pseudoviruses with Engineered Variant Human ACE2 Protein Comprising Amino Acid Substitutions Designed to Increase Binding Affinity

This example demonstrates that engineered variant human ACE2 protein comprising amino acid substitutions designed to increase binding affinity to the SARS-CoV-2 spike (S) glycoprotein can result in improved neutralization against certain SARS-CoV-2 variants.

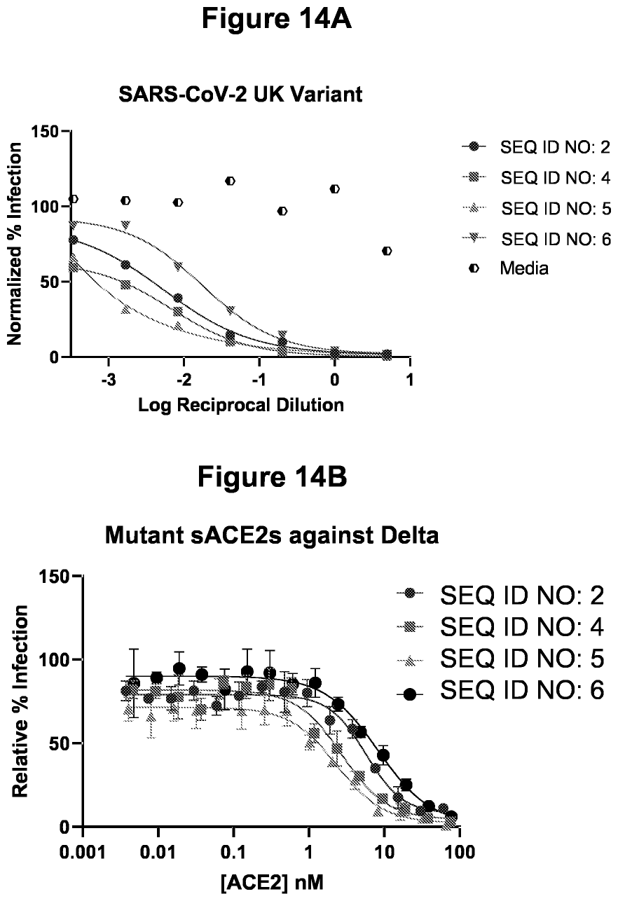

To further assess the neutralization efficacy of the engineered variant human ACE2 polypeptides generated in Example 1 (SEQ ID NOs: 4, 5 and 6), an RVP neutralization assay as described in Example 10 was performed using two RVPs displaying the alpha (UK) and delta (India) S glycoprotein variants, respectively. A polypeptide with the wild-type amino acid sequence of the extracellular domain of human ACE2 (SEQ ID NO:2) served as a control. The variant ACE2 polypeptide encoded by SEQ ID NO: 6 performed similarly to the polypeptide with the wild-type amino acid sequence (SEQ ID NO:2). The variant polypeptide encoded by SEQ ID NO:6 displayed an IC50 of 4.956 nM when incubated with an RVP expressing the S glycoprotein of the UK variant, which is comparable to the IC50 value of 4.241 nM for wildtype human ACE2 protein. Variant polypeptides comprising amino acid substitutions N90D and R273A or N90D and T92A, respectively (encoded by SEQ ID NOs: 4 and 5, respectively) demonstrated more effective neutralization of UK variant S glycoprotein RVPs, displaying IC50s of 1.746 nM and 2.075 nM, respectively (Table 7, FIG. 14A).

TABLE 7
Estimated IC50 of variant human ACE2 polypeptides against
RVPs displaying the UK variant S glycoprotein

| Polypeptide-encoding | Preliminary Estimate |
|---|---|
| sequence | of IC50 (nM) |
| SEQ ID NO: 2 | 4.241 |
| SEQ ID NO: 4 | 1.746 |
| SEQ ID NO: 5 | 2.075 |
| SEQ ID NO: 6 | 4.956 |

The polypeptides encoded by SEQ ID NOs: 4 and 5 also demonstrated more effective neutralization of delta variant S glycoprotein RVPs (Table 8, FIG. 14B), displaying IC50 values of 2.740 nM and 2.335 nM, respectively. This compared favourably to the IC50 value of 5.715 nM obtained with the polypeptide comprising the wildtype amino acid sequence encoded by SEQ ID NO:2 and the IC50 value of 8.288 obtained with the variant polypeptide encoded by SEQ ID NO: 6.

Overall, these data confirm a general trend that was also observed in Example 10. In that example, the variant ACE2 polypeptides encoded by SEQ ID NOs: 4 and 5 were slightly more effective in neutralizing the Wuhan and D614G variant S glycoprotein RVPs than the ACE2 polypeptides encoded by SEQ ID NOs: 2 and 6. In this example, SEQ ID NOs: 4 and 5 displayed improved IC50 values against the alpha (UK) and delta (India) variants compared to wildtype human ACE2 protein.

TABLE 8
Estimated IC50 of variant human ACE2 polypeptides against
RVPs displaying the delta variant S glycoprotein

| Polypeptide-encoding | Preliminary Estimate |
|---|---|
| sequence | of IC50 (nM) |
| SEQ ID NO: 2 | 5.715 |
| SEQ ID NO: 4 | 2.740 |
| SEQ ID NO: 5 | 2.335 |
| SEQ ID NO: 6 | 8.288 |

This example demonstrates that engineered variant ACE2 polypeptides can present options for future generations of therapeutics with increased levels of efficacy against current circulating variants of SARS-CoV-2—particularly when combined with the increased avidity afforded by an Fc fusion protein, as exemplified in Example 12.

### Example 12. Effective Neutralization of S Glycoprotein-Displaying Pseudoviruses with Polypeptides Comprising the Extracellular Domain of Human ACE2 Protein and an Immunoglobulin Fc Region

This example demonstrates the efficacy of an exemplary polypeptide comprising the extracellular domain of human ACE2 protein and an immunoglobulin Fc region (ACE2-Fc fusion protein) for neutralizing RVPs. Such a polypeptide can effectively neutralize pseudotyped reporter virus particles (RVPs) displaying the SARS-CoV-2 spike (S) glycoprotein in a cell culture model of infection.

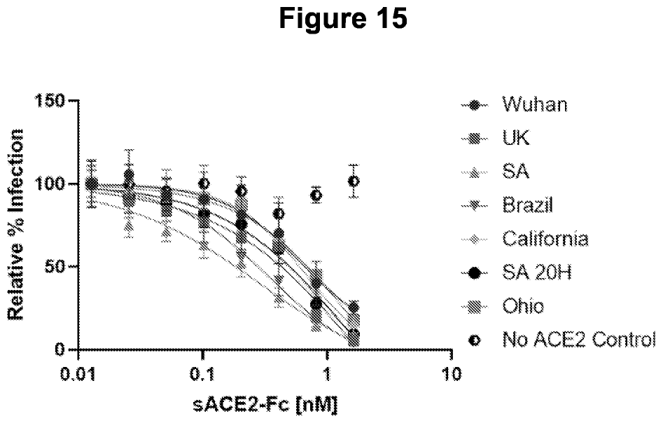

In order to assess the potential increased neutralization from an sACE2-Fc fusion protein, a set of experiments were conducted using the protocol from Example 10, but with the sACE2-Fc fusion protein encoded by SEQ ID NO: 3. Wuhan, UK, SA, Brazil, California, and Ohio S glycoprotein variant RVPs were included for analysis. The sACE2-Fc fusion protein encoded by SEQ ID NO: 3 demonstrated broad neutralization of all RVPs, with IC50s ranging from 0.6986 nM for the Ohio S glycoprotein variant to 0.1840 nM for the South African S glycoprotein variant. (FIG. 15) These data taken together indicate the potential for a variant agnostic treatment or prophylactic approach to controlling SARS-CoV-2 infection using an sACE2-Fc fusion protein.

TABLE 9
Estimated IC50 of encoded sACE2-Fc fusion protein against
RVPs displaying various variant S glycoproteins

| Variant | IC50 (nM) |
|---|---|
| Wuhan-Hu-1 (D614G) | 0.6657 |
| UK (Alpha) (B.1.1.7) | 0.4283 |
| South Africa (Beta) | 0.1840 |
| (B.1.351) | |
| Brazil (Gamma) (P.1) | 0.2784 |
| California 452R | 0.6001 |
| South Africa 20H | 0.4989 |
| Ohio | 0.6986 |

To confirm these findings, a second set of experiments was conducted using a different reporter virus particle (RVP) system using green fluorescent protein (GFP) as the reporter in place of luciferase. Furthermore, RVPs displaying additional S glycoprotein variants that had emerged in the meantime were tested in this experiment. In the absence of a specific neutralizing agent, the RVPs infect engineered HEK293 cells expressing ACE2 on their cell surface, resulting in expression of GFP, observed 48-72 hours after the RVP is exposed to the cells. To assess neutralization, the RVPs were first incubated with varying concentrations of the sACE2-Fc fusion protein encoded by SEQ ID NO: 3. Subsequently, the RVPs were incubated with the HEK293 cells and the number of GFP positive cells was determined. The number of GFP positive cells is directly proportional to RVP infection levels, and as such, a lower number of GFP positive cells indicates successful neutralization by the sACE2 protein.

TABLE 10
Estimated IC50 of encoded sACE2-Fc fusion protein against
RVPs displaying various variant S glycoproteins

| Variant | IC50 (nM) |
|---|---|
| Wuhan-Hu-1 (D614G) | 1.151 |
| Alpha (B.1.1.7) | 0.5633 |
| Beta (B.1.351) | 0.3094 |
| Gamma (P.1) | 0.2286 |
| Delta (B.1.617.2) | 0.3059 |
| Eta (B.1.525) | 0.4507 |
| Iota (B.1.526) | 0.9985 |

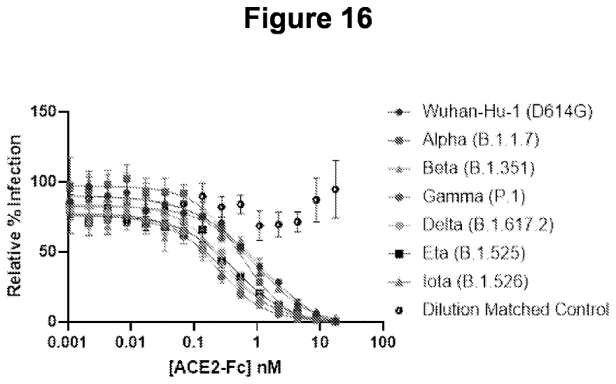

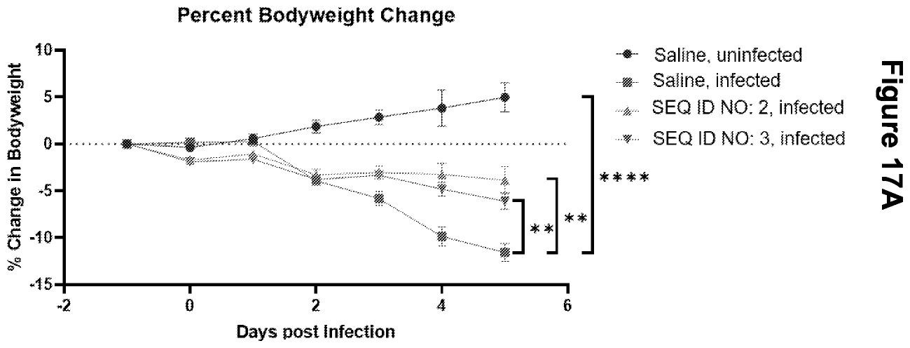

sACE2-Fc encoded by SEQ ID NO: 3 demonstrated broad neutralization of various RVPs displaying different variant S glycoproteins, with IC50s ranging from 0.2286 nM for the Gamma variant to 1.151 nM for the Wuhan-Hu-1 variant (Table 10, FIG. 16). As expected with different assays, slight differences in IC50s were observed with the GFP reporter in this second set of experiments, compared to the luciferase reporter in the first set of experiments. Taken together, these data confirm the potential for a variant agnostic treatment or prophylactic approach to controlling SARS-CoV-2 infection using an sACE2-Fc fusion protein.

As exemplified in Example 8—in particular FIG. 11H—attaching the Fc domain causes the ACE2 molecule to dimerize. Dimerization of the ACE2 molecule results in increased avidity. Without wishing to be bound by any particular theory, the inventors believe that the increased avidity resulting from the fusion of sACE2 to the Fc protein yields the improved IC50 values in the experiments described in this example compared to the experiments described in Example 11. These data support the notion that fusing the engineered variant human ACE2 polypeptides tested in Example 11 (in particular those encoded by SEQ ID NO: 4 and 5) to an Fc region (e.g., the one encoded by SEQ ID NO: 27) to yield an ACE2-fusion protein similar to that tested in this example would result in a more effective neutralization of viruses that must bind to ACE2 protein in order to gain entry into cells (e.g., various SARS-CoV2 variants, as exemplified herein).

### Example 13. Protective and Treatment Efficacy Against COVID-19 in Syrian Golden Hamster Challenge Model

This example demonstrates that sequence-optimized mRNA-encoded polypeptides comprising the extracellular domain of human ACE2 protein can be used to protect against or reduce infection with a virus which uses the ACE2 protein for cellular entry, thereby preventing severe lung pathology.

SARS-CoV-2 infection in Syrian golden hamster is a disease model, where the viral infection is associated with high levels of virus replication with peak titers in the lungs and nasal epitheliums at 2 day post infection (DPI), histopathological evidence of disease in lungs at 7 DPI, and about 8-15% weight loss around 7 DPI. To evaluate the efficacy of the sequence-optimized mRNAs of the invention, Syrian golden hamsters were dosed 1 day before viral challenge with 30 μg sACE2 or sACE2-Fc encoding mRNAs formulated into LNPs intratracheally (SEQ ID NO: 2 and SEQ ID NO: 3, respectively), or with saline as a control. The LNPs comprised SY-3-E14-DMAPr, DOPE, cholesterol and DMG-PEK2K at molar ratios of 50:15:30:5. The N/P ratio was about 4. Animals were challenged intranasally on day 0 with 5×10⁴ PFU of SARS-CoV-2 (Washington variant) per nostril (1×10⁵ PFU total/animal). Animals were monitored for clinical observations and body weight daily. A number of animals in each group were sacrificed on day 3 and viral titer was measured (TCID50).

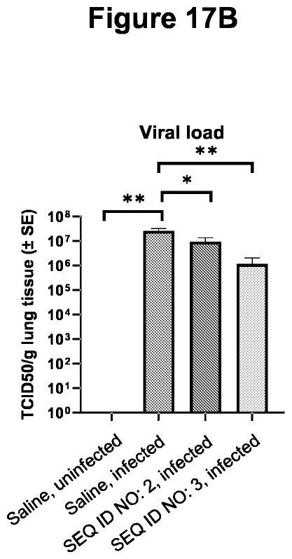

Infected hamsters showed a reduction in bodyweight over the five days following infection, whereas uninfected hamsters showed an increase in bodyweight. Notably, the decrease in bodyweight was statistically significantly less for hamsters treated with LNPs comprising mRNAs encoding SEQ ID NO: 2 or SEQ ID NO: 3 compared to the control infection hamsters (FIG. 17A). The data presented in FIG. 17A indicate that pretreatment with sACE2- and sACE2-Fc-encoding mRNAs can minimize the loss of bodyweight resulting from infection with SARS-CoV-2. Consistent with the in vitro data in Example 12, there was a trend towards greater efficacy of the sACE2-Fc-encoding mRNA.

The viral titer in animals sacrificed on day 3 was significantly lower for animals dosed with sACE2 or sACE2-Fc compared to the control infected animals (FIG. 17B). In particular, the animals dosed with the sACE2-Fc fusion showed a 10-fold decrease in TCID50 compared to the control infected animals. This finding is consistent with the effect observed in Examples 11 and 12 that the Fc fusion increases the avidity of the sACE2 through dimerization, resulting in an EC50 for the S glycoprotein that is increased by an order of magnitude, and therefore is more effective in neutralizing SARS-CoV-2.

This example provides in vivo viral challenge study data demonstrating that pulmonary delivery of sequence-optimized mRNAs of the invention encoding sACE2 and sACE2-Fc reduces the viral load and symptoms, such as bodyweight loss, after SARS-CoV-2 infection in a large rodent model that is suitable for viral challenge studies. A decrease in viral load early in the infection with SARS-CoV2 is most critical in improving the overall health of an infected patient. Therefore, these data validate the therapeutic approach disclosed in the present application.

1. (canceled)

2. An mRNA comprising an optimized nucleotide sequence encoding a polypeptide comprising the extracellular domain of a human angiotensin-converting enzyme 2 (ACE2) protein or a portion thereof that binds to a viral surface protein, wherein the coding region of the optimized nucleotide sequence consists of codons associated with a usage frequency greater than or equal to 10%; and wherein the optimized nucleotide sequence:
(a) does not contain a termination signal having one of the following nucleotide sequences:
5′-X₁AUCUX₂UX₃-3′, wherein X₁, X₂ and X₃ are independently selected from A, C, U or G; and
5′-X₁AUCUX₂UX₃-3′, wherein X₁, X₂ and X₃ are independently selected from A, C, U or G;
(b) does not contain any negative cis-regulatory elements and negative repeat elements;
(c) has a codon adaptation index (CAI) greater than 0.8; and
when the optimized nucleotide sequence is divided into non-overlapping 30 nucleotide-long portions, each portion of the optimized nucleotide sequence has a guanine cytosine content ranging from 30%-70%.

3. The mRNA of claim 2, wherein the optimized nucleotide sequence does not contain a termination signal having one of the following sequences:
UAUCUGUU;
UUUUUU;
AAGCUU;
GAAGAGC;
UCUAGA.

4. The mRNA of claim 2, wherein the extracellular domain of the human ACE2 protein or portion thereof comprises
(a) amino acid residues 1-740 of a naturally occurring human ACE2 protein encoded by SEQ ID NO: 10, and/or
(b) one or more amino acid substitutions relative to the amino acid sequence of SEQ ID NO: 10.

5. The mRNA of claim 2, wherein the polypeptide comprises two extracellular domains of the human ACE2 protein or portions thereof operably linked to form a dimer.

6. The mRNA of claim 2, wherein the polypeptide further comprises
(a) an immunoglobulin Fc region linked to the C-terminus of the extracellular domain of the human ACE2 protein or portion thereof, and/or
(b) a C-terminal localization sequence that is exogenous to the naturally occurring human ACE2 protein encoded by SEQ ID NO: 10.

7. The mRNA of claim 6, wherein the immunoglobulin Fc region is derived from IgG1, IgG2, or IgG4.

8-10. (canceled)

11. The mRNA of claim 6, wherein the extracellular domain of the human ACE2 protein or portion thereof, the C-terminal localization sequence, and/or the immunoglobulin Fc region within the polypeptide are linked by one or more linker sequences, e.g., GGGGS.

12. (canceled)

13. The mRNA of claim 4, wherein the one or more amino acid substitutions
increase the binding affinity of the human ACE2 protein for a SARS-CoV-2 spike (S) glycoprotein,
inactivate the catalytic activity of the human ACE2 protein and/or affect the glycosylation of the human ACE2 protein, and/or
are at any one of the positions selected from positions 90, 92, 273, 374, and 378 of SEQ ID NO: 10.

14-15. (canceled)

16. The mRNA of claim 13, wherein the one or more amino acid substitutions are selected from the group consisting of: N90D, T92A, R273A, H374N, and H378N.

17. The mRNA of claim 16, wherein the one or more amino acid substitutions comprise or are
(a) N90D and R273A,
(b) N90D and T92A, or
(c) H374N and H378N.

18-19. (canceled)

20. The mRNA of claim 2, wherein the amino acid sequence of the polypeptide is at least 90%, 95%, 96%, 97%, 98%, or 99% identical to, comprises or consists of the amino acid sequence of any one of SEQ ID NOs: 11 to 18, 34 and 35.

21-29. (canceled)

30. The mRNA of claim 2, wherein the optimized nucleotide sequence is at least 60%, 65%, 70%, 75%, 80%, 85%, 90%, 95%, 96%, 97%, 98%, or 99% identical to, comprises, or consists of
the nucleic acid sequence of SEQ ID NO: 2 and encodes the amino acid sequence of SEQ ID NO: 11,
the nucleic acid sequence of SEQ ID NO: 3 and encodes the amino acid sequence of SEQ ID NO: 12,
the nucleic acid sequence of SEQ ID NO: 4 and encodes the amino acid sequence of SEQ ID NO: 13,
the nucleic acid sequence of SEQ ID NO: 5 and encodes the amino acid sequence of SEQ ID NO: 14,
the nucleic acid sequence of SEQ ID NO: 6 and encodes the amino acid sequence of SEQ ID NO: 15,
the nucleic acid sequence of SEQ ID NO: 7 and encodes the amino acid sequence of SEQ ID NO: 16,
the nucleic acid sequence of SEQ ID NO: 8 and encodes the amino acid sequence of SEQ ID NO: 17, or
the nucleic acid sequence of SEQ ID NO: 9 and encodes the amino acid sequence of SEQ ID NO: 18.

31-37. (canceled)

38. The mRNA of claim 2,
(a) wherein the 5′ cap has the following structure: 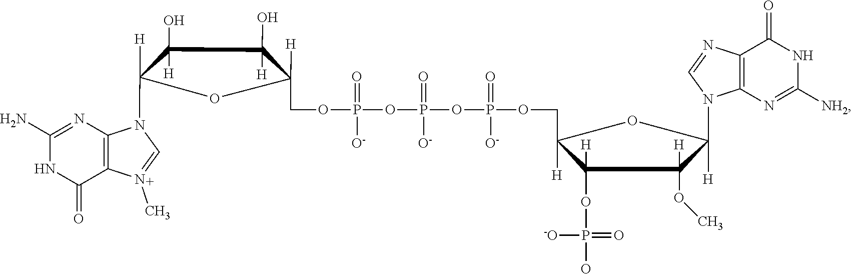
(b) wherein the tail structure of the mRNA
(i) comprises a poly(A) tail or a poly(C) tail,
(ii) comprises at least 50 adenosine or cytosine nucleotides, and/or
(iii) is approximately 100-500 nucleotides in length.

39-42. (canceled)

43. The mRNA of claim 2, wherein the mRNA comprises
(a) a 5′ untranslated region (5′ UTR) different than the naturally occurring 5′ UTR of a human ACE2 mRNA,
(b) a 3′ untranslated region (3′ UTR) different than the naturally occurring 3′ UTR of a human ACE2 mRNA, and/or
(c) one or more modified nucleosides.

44. (canceled)

45. The mRNA of claim 43, wherein
(a) the 5′ UTR has the nucleotide sequence of SEQ ID NO: 19,
(b) the 3′ UTR has the nucleotide sequence of SEQ ID NO: 20 or SEQ ID NO: 21, and/or
(c) the one or more modified nucleosides is a nucleoside analog selected from the group consisting of: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine (e.g., N-1-methyl-pseudouridine), 2-thiouridine, and 2-thiocytidine.

46-48. (canceled)

49. The mRNA of claim 2 for use in therapy.

50. A DNA vector encoding the mRNA of claim 2, optionally wherein the DNA vector further comprises a promoter and/or a terminator, further optionally wherein the promoter is a SP6 RNA polymerase promoter or a T7 RNA polymerase promoter.

51-53. (canceled)

54. A lipid nanoparticle (LNP) encapsulating the mRNA of claim 2, optionally wherein the lipid component of the lipid nanoparticle consists of a cationic lipid, a non-cationic lipid, a PEG-modified lipid, and optionally a cholesterol-based lipid, further optionally wherein
the cationic lipid constitutes about 30-60% of the lipid nanoparticle by molar ratio, e.g., about 35-40%,
the ratio of cationic lipid to non-cationic lipid to cholesterol-based lipid to PEG-modified lipid is approximately 30-60:25-35:20-30:1-15 by molar ratio or the ratio of cationic lipid to non-cationic lipid to PEG-modified lipid is approximately 55-65:30-40:1-15 by molar ratio,
the lipid nanoparticles have an average size of less than 150 nm, e.g., less than 100 nm, e.g., about 50-70 nm,
the mRNA is present at a concentration of between about 0.5 mg/mL to about 1.0 mg/mL,
the lipid nanoparticles are suspended in an aqueous solution comprising trehalose, e.g., 10% (w/v) trehalose in water, or sucrose, e.g., 10% (w/v) sucrose in water.

55. (canceled)

56. The lipid nanoparticle (LNP) of claim 54, wherein:
(a) the cationic lipid is selected from cKK-E12, cKK-E10, HGT5000, HGT5001, ICE, HGT4001, HGT4002, HGT4003, TLI-OID-DMA, TL1-04D-DMA, TL1-08D-DMA, TL1-10D-DMA, OF-Deg-Lin, OF-02, GL-TES-S A-DMP-E 18-2, GL-TES-SA-DME-E18-2, SY-3-E14-DMAPr, TL1-10D-DMA, HEP-E3-E10, HEP-E4-E10, RL3-DMA-07D, RL2-DMP-07D, cHse-E-3-E10, cHse-E-3-E12, cDD-TE-4-E12, SI-4-E 14-DMAP r, TL-1-12D-DMA, SY-010, and SY-011;
(b) the non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DEPE 1,2-dierucoyl-sn-glycero-3-phosphoethanolamine, DOPC (1,2-dioleyl-sn-glycero-3-phosphotidylcholine), DPPE (1,2-dipalmitoyl-sn-glycero-3-phosphoethanolamine), DMPE (1,2-dimyristoyl-sn-glycero-3-phosphoethanolamine), and DOPG(1,2-dioleoyl-sn-glycero-3-phospho-(T-rac-glycerol));
(c) the PEG-modified lipid is DMG-PEG-2K, and/or
(d) the cholesterol-based lipid is cholesterol.

57-76. (canceled)

77. A composition comprising the lipid nanoparticles (LNP) of claim 54, optionally wherein the composition is a pharmaceutical composition.

78-83. (canceled)

84. A kit comprising the composition of claim 77.

85. A method of treating or preventing an infection in a subject caused by a virus, or a method of neutralizing a virus, which uses human ACE2 protein for cellular entry, wherein the method comprises administering the composition of claim 77 to the subject, optionally wherein said virus is a coronavirus, e.g. a beta-coronavirus, e.g., SARS-CoV-2.

86-123. (canceled)

124. A method of treating or preventing an infection in a subject caused by a virus, or a method of neutralizing a virus, which uses human ACE2 protein for cellular entry, wherein the method comprises administering the composition of claim 77 to the subject intravenously or by nebulization.

125-134. (canceled)

135. A dry powder formulation comprising a plurality of spray-dried particles comprising the lipid nanoparticle (LNP) of claim 54 and one or more polymers.

136-137. (canceled)


SEQUENCE LISTING (35 sequences)

SEQ ID NO: 1 - RNA, 3249 nt, Homo sapiens
augucaagcucuuccuggcuccuucucagccuuguugcuguaacugcugcucaguccaccauugaggaacaggccaagacauuuuuggacaaguuuaaccacgaagccgaagaccuguucuaucaaaguucacuugcuucuuggaauuauaacaccaauauuacugaagagaauguccaaaacaugaauaaugcuggggacaaauggucugccuuuuuaaaggaacaguccacacuugcccaaauguauccacuacaagaaauucagaaucucacagucaagcuucagcugcaggcucuucagcaaaaugggucuucagugcucucagaagacaagagcaaacgguugaacacaauucuaaauacaaugagcaccaucuacaguacuggaaaaguuuguaacccagauaauccacaagaaugcuuauuacuugaaccagguuugaaugaaauaauggcaaacaguuuagacuacaaugagaggcucugggcuugggaaagcuggagaucugaggucggcaagcagcugaggccauuauaugaagaguauguggucuugaaaaaugagauggcaagagcaaaucauuaugaggacuauggggauuauuggagaggagacuaugaaguaaaugggguagauggcuaugacuacagccgcggccaguugauugaagauguggaacauaccuuugaagagauuaaaccauuauaugaacaucuucaugccuaugugagggcaaaguugaugaaugccuauccuuccuauaucaguccaauuggaugccucccugcucauuugcuuggugauaugugggguagauuuuggacaaaucuguacucuuugacaguucccuuuggacagaaaccaaacauagauguuacugaugcaaugguggaccaggccugggaugcacagagaauauucaaggaggccgagaaguucuuuguaucuguuggucuuccuaauaugacucaaggauucugggaaaauuccaugcuaacggacccaggaaauguucagaaagcagucugccaucccacagcuugggaccuggggaagggcgacuucaggauccuuaugugcacaaaggugacaauggacgacuuccugacagcucaucaugagauggggcauauccaguaugauauggcauaugcugcacaaccuuuucugcuaagaaauggagcuaaugaaggauuccaugaagcuguuggggaaaucaugucacuuucugcagccacaccuaagcauuuaaaauccauuggucuucugucacccgauuuucaagaagacaaugaaacagaaauaaacuuccugcucaaacaagcacucacgauuguugggacucugccauuuacuuacauguuagagaaguggagguggauggucuuuaaaggggaaauucccaaagaccaguggaugaaaaaguggugggagaugaagcgagagauaguuggggugguggaaccugugccccaugaugaaacauacugugaccccgcaucucuguuccauguuucuaaugauuacucauucauucgauauuacacaaggacccuuuaccaauuccaguuucaagaagcacuuugucaagcagcuaaacaugaaggcccucugcacaaaugugacaucucaaacucuacagaagcuggacagaaacuguucaauaugcugaggcuuggaaaaucagaacccuggacccuagcauuggaaaauguuguaggagcaaagaacaugaauguaaggccacugcucaacuacuuugagcccuuauuuaccuggcugaaagaccagaacaagaauucuuuugugggauggaguaccgacuggaguccauaugcagaccaaagcaucaaagugaggauaagccuaaaaucagcucuuggagauaaagcauaugaauggaacgacaaugaaauguaccuguuccgaucaucuguugcauaugcuaugaggcaguacuuuuuaaaaguaaaaaaucagaugauucuuuuuggggaggaggaugugcgaguggcuaauuugaaaccaagaaucuccuuuaauuucuuugucacugcaccuaaaaaugugucugauaucauuccuagaacugaaguugaaaaggccaucaggaugucccggagccguaucaaugaugcuuuccgucugaaugacaacagccuagaguuucuggggauacagccaacacuuggaccuccuaaccagcccccuguuuccauauggcugauuguuuuuggaguugugaugggagugauagugguuggcauugucauccugaucuucacugggaucagagaucggaagaagaaaaauaaagcaagaaguggagaaaauccuuaugccuccaucgauauuagcaaaggagaaaauaauccaggauuccaaaacacugaugauguucagaccuccuuuuagaaaaaucuauguuuuuccucuugaggugauuuuguuguauguaaauguuaauuucaugguauagaaaauauaagaugauaaagauaucauuaaaugucaaaacuaugacucuguucagaaaaaaaauuguccaaagacaacauggccaaggagagagcaucuucauugacauugcuuucaguauuuauuucugucucuggauuugacuucuguucuguuucuuaauaaggauuuuguauuagaguauauuagggaaaguguguauuuggucucacaggcuguucagggauaaucuaaauguaaaugucuguugaauuucugaaguugaaaacaaggauauaucauuggagcaaguguuggaucuuguauggaauauggauggaucacuuguaaggacagugccugggaacugguguagcugcaaggauugagaauggcaugcauuagcucacuuucauuuaauccauugucaaggaugacaugcuuucuucacaguaacucaguucaaguacuauggugauuugccuacagugauguuuggaaucgaucaugcuuucuucaaggugacaggucuaaagagagaagaauccagggaacagguagaggacauugcuuuuucacuuccaaggugcuugaucaacaucucccugacaacacaaaacuagagccaggggccuccgugaacucccagagcaugccugauagaaacucauuucuacuguucucuaacuguggagugaauggaaauuccaacuguauguucacccucugaaguggguacccagucucuuaaaucuuuuguauuugcucacaguguuugagcagugcuga

SEQ ID NO: 2 - RNA, 2223 nt, Artificial Sequence
  RNA sequence encoding hsACE2
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcuga

SEQ ID NO: 3 - RNA, 2931 nt, Artificial Sequence
  RNA sequence encoding hsACE2-GS-Fc
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaaguga

SEQ ID NO: 4 - RNA, 2223 nt, Artificial Sequence
  RNA sequence encoding hsACE2 N90D R273A
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucaggaccugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcgccuuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcuga

SEQ ID NO: 5 - RNA, 2223 nt, Artificial Sequence
  RNA sequence encoding hsACE2 N90D T92A
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucaggaccuggccgugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcuga

SEQ ID NO: 6 - RNA, 2223 nt, Artificial Sequence
  RNA sequence encoding hsACE2 H374N H378N
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccauaacgagaugggcaauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcuga

SEQ ID NO: 7 - RNA, 3006 nt, Artificial Sequence
  RNA sequence encoding hsACE2-GS-Fc-GS-KLKL
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaagggcggagggggaagccagaggaacccaaagcugaaacugauucgcagacacccuacccugcgcaucccacccauuuga

SEQ ID NO: 8 - RNA, 3006 nt, Artificial Sequence
  RNA sequence encoding hsACE2-GS-Fc-GS-RLRL
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaagggcggagggggaagccagcgcaauccucgccugcggcugauccggagacauccuacacugagaauucccccaauuuga

SEQ ID NO: 9 - RNA, 3357 nt, Artificial Sequence
  RNA sequence encoding hsACE2-GS-Fc-GS-J chain
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucagaaucugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaagggcggagggggaagccaggaggacgagcgcaucguucugguggauaacaagugcaaguccgcuaggaucacuuccaggaucauuagguccuccgaggacccaaaugaagauauuguggagcggaacauucggauuauuguuccccugaacaaucgggagaauaucagcgacccuaccuccccacucagaaccagauuuguguaucaccugagugaccuguccaagaagugcgaucccaccgaaguggagcuggauaaucagauugugaccgcuacucagucuaacaucugcgacgaagauuccgccacagaaacaugcuauacauacgauagaaacaaguguuacacugccguggucccucugguguacggcggagaaaccaagaugguggagaccgcccugaccccagacgcauguuacccagacuga

SEQ ID NO: 10 - protein, 805 aa, Artificial Sequence [sequence omitted]

SEQ ID NO: 11 - protein, 740 aa, Artificial Sequence
  hsACE2
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 12 - protein, 976 aa, Artificial Sequence
  hsACE2-GS-Fc
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 740 aa, Artificial Sequence
  hsACE2 N90D R273A
MSSSSWLLLSLVAVTAAQSTIEEQAKTFLDKFNHEAEDLFYQSSLASWNYNTNITEENVQNMNNAGDKWSAFLKEQSTLAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 14 - protein, 740 aa, Artificial Sequence
  hsACE2 N90D T92A
MSSSSWLLLSLVAVTAAQSTIEEQAKTFLDKFNHEAEDLFYQSSLASWNYNTNITEENVQNMNNAGDKWSAFLKEQSTLAQMYPLQEIQDLAVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 15 - protein, 740 aa, Artificial Sequence
  hsACE2 H374N H378N
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 16 - protein, 1001 aa, Artificial Sequence
  hsACE2-GS-Fc-GS-KLKL
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQRNPKLKLIRRHPTLRIPPI

SEQ ID NO: 17 - protein, 1001 aa, Artificial Sequence
  hsACE2-GS-Fc-GS-RLRL
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQRNPRLRLIRRHPTLRIPPI

SEQ ID NO: 18 - protein, 1118 aa, hsACE2-GS-Fc-GS-J chain
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQEDERIVLVDNKCKSARITSRIIRSSEDPNEDIVERNIRIIVPLNNRENISDPTSPLRTRFVYHLSDLSKKCDPTEVELDNQIVTATQSNICDEDSATETCYTYDRNKCYTAVVPLVYGGETKMVETALTPDACYPD

SEQ ID NO: 19 - RNA, 140 nt, 5′ UTR sequence
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 20 - RNA, 105 nt, Artificial Sequence
  3′ UTR sequence
cggguggcaucccugugaccccuccccagugccucuccuggcccuggaaguugccacuccagugcccaccagccuuguccuaauaaaauuaaguugcaucaagcu

SEQ ID NO: 21 - RNA, 105 nt, Artificial Sequence
  3′ UTR sequence
ggguggcaucccugugaccccuccccagugccucuccuggcccuggaaguugccacuccagugcccaccagccuuguccuaauaaaauuaaguugcaucaaagcu

SEQ ID NO: 22 - RNA, 693 nt, Artificial Sequence
  RNA sequence encoding immunoglobulin Fc region from human IgG1
ccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaag

SEQ ID NO: 23 - RNA, 15 nt, Artificial Sequence
  RNA sequence encoding GS linker
ggcggagggggaagc

SEQ ID NO: 24 - RNA, 60 nt, Artificial Sequence
  RNA sequence encoding KLKL localization sequence
cagaggaacccaaagcugaaacugauucgcagacacccuacccugcgcaucccacccauu

SEQ ID NO: 25 - RNA, 60 nt, Artificial Sequence
  RNA sequence encoding RLRL localization sequence
cagcgcaauccucgccugcggcugauccggagacauccuacacugagaauucccccaauu

SEQ ID NO: 26 - RNA, 411 nt, Artificial Sequence
  RNA sequence encoding immunoglobulin J chain
caggaggacgagcgcaucguucugguggauaacaagugcaaguccgcuaggaucacuuccaggaucauuagguccuccgaggacccaaaugaagauauuguggagcggaacauucggauuauuguuccccugaacaaucgggagaauaucagcgacccuaccuccccacucagaaccagauuuguguaucaccugagugaccuguccaagaagugcgaucccaccgaaguggagcuggauaaucagauugugaccgcuacucagucuaacaucugcgacgaagauuccgccacagaaacaugcuauacauacgauagaaacaaguguuacacugccguggucccucugguguacggcggagaaaccaagaugguggagaccgcccugaccccagacgcauguuacccagac

SEQ ID NO: 27 - protein, 231 aa, Artificial Sequence
  Immunoglobulin Fc region from human IgG1
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 5 aa, Artificial Sequence
  GS linker
GGGGS

SEQ ID NO: 29 - protein, 20 aa, Artificial Sequence
  KLKL localization sequence
QRNPKLKLIRRHPTLRIPPI

SEQ ID NO: 30 - protein, 20 aa, Artificial Sequence
  RLRL localization sequence
QRNPRLRLIRRHPTLRIPPI

SEQ ID NO: 31 - protein, 137 aa, Artificial Sequence
  Immunoglobulin J chain
QEDERIVLVDNKCKSARITSRIIRSSEDPNEDIVERNIRIIVPLNNRENISDPTSPLRTRFVYHLSDLSKKCDPTEVELDNQIVTATQSNICDEDSATETCYTYDRNKCYTAVVPLVYGGETKMVETALTPDACYPD

SEQ ID NO: 32 - RNA, 2945 nt, Artificial Sequence
  RNA sequence encoding hsACE2-GS-Gc wherein the hsACE2 is hsACE2 N90D R273A
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucaggaccugacagugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcgccuuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcugaggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaagugaccgugagcuga

SEQ ID NO: 33 - RNA, 2945 nt, Artificial Sequence
  RNA sequence encoding hsACE2-GS-Fc wherein the hsACE2 is hsACE2 N90D T92A
augucuuccucuucuuggcugcugcugagccugguggccgucacagccgcucaguccacaaucgaggaacaggcuaaaacuuuccuggacaaauucaaccacgaagcagaggaccucuucuaucagucuagccuggccuccuggaacuacaacaccaacauuacugaggagaacgugcagaacaugaacaacgccggagacaagugguccgccuuccugaaggagcagucuacgcuggcacagauguacccacugcaggagauucaggaccuggccgugaagcugcagcuucaggcucugcagcagaacgggucuagugugcugucugaggacaaaucuaagagguugaacaccauucugaauacaauguccacaaucuauagcaccggcaaggugugcaauccugauaauccucaggagugccugcugcuggagccaggacugaaugaaauuauggcuaacucccuggauuacaacgaaagacugugggccugggagagcuggagguccgaggugggaaagcagcugagaccucuguaugaggaguaugugguccugaagaaugagauggcccgcgccaaccacuacgaagacuauggagacuacuggagaggagacuaugaggugaacggaguggauggauaugacuacagcaggggccagcugaucgaagauguggaacauaccuucgaggagaucaagccccuguaugagcaucugcacgcauacgucagggcaaagcugaugaacgccuaucccuccuacaucagcccaaucgggugucugcccgcucaccuccugggggauauguggggcagguuuuggaccaaucucuacucucugacuguccccuuuggccagaagccuaacaucgacgugacagaugcaaugguggaccaggcaugggacgcccagaggaucuucaaggaagcagagaaauucuucguguccgugggacugccaaauaugacccagggcuuuugggagaauagcaugcugaccgacccagggaacgugcagaaggcugugugucacccuacagccugggaucuggggaagggcgacuucaggauccugauguguacaaagguuacaauggacgauuuccugacagcccaucacgagaugggccauauccaguacgacauggccuaugcugcucagccuuuucugcugaggaauggagcaaaugagggguuccaugaggccgugggagagauuaugucccugucugcagcuaccccuaaacaccugaaaagcaucggccugcugucaccagacuuccaggaggacaacgagacagagaucaacuuucugcugaagcaggcacugacaaucguggggacacugccuuucaccuauaugcuggaaaaguggagguggaugguguucaagggcgaaauuccuaaggaccaguggaugaagaaauggugggagaugaaaagggagauugugggcgugguggagccugugccucacgaugagacuuauugugaccccgccagccuguuucacgucuccaacgacuacuccuucaucagauauuacacaagaacucuguaccaguuccaguuccaggaggcccugugucaggccgccaaacacgaggggccacugcauaaaugcgauauuagcaacagcaccgaggccggccagaagcucuuuaauaugcugaggcugggcaaguccgagccuuggacucuagcccuggagaacguggugggagcaaagaacaugaaugugagaccacugcugaacuacuucgagccccuguuuaccuggcugaaagaccagaacaaaaacucuuuugugggcuggagcaccgauuggucuccauacgcagaccagagcaucaaaguccgcaucucccugaaaucagcccugggggauaaggccuaugaguggaaugacaaugaaauguaccucuuucgguccagcguggcauaugcuaugcggcaguacuuccugaaggucaagaaccagaugauccuguuuggcgaggaagacgugaggguugccaaucugaagccccgcauuucuuuuaacuuuuucgucacugcccccaagaacgugagcgacaucauuccaaggaccgaaguggaaaaggcuaucagaaugucucgguccaggaucaacgacgccuucagguugaacgacaacagccuggaguuucuggggauucagccuacacuggggccucccaaucagccacccgugagcugaggcggagggggaagcccgaagucuugcgauaaaacccacacauguccuccauguccagcacccgaguuacugggcggcccuagcguguuccuguuuccaccuaagccaaaggauacacugaugaucucacggacaccugaggugacuuguguugugguggacgugucucaugaggacccugaggugaaauucaacugguacguggacggcguggaggugcacaacgccaaaaccaagcccagggaggagcaguacaacuccaccuacaggguggugucuguguugacagugcugcaucaggacuggcugaacgggaaggaguauaaguguaagguguccaauaaggcccuccccgccccuaucgaaaagacaaucagcaaggcuaagggacagcccagggagccacagguguauacucugccaccuucacgggaugaacugaccaagaaucaggugucucugaccugccuggugaagggguuuuacccauccgacaucgccgucgaaugggaaagcaauggccaacccgagaacaauuauaagacaaccccaccaguucuggacuccgauggauccuucuuccuguacagcaagcugacuguggauaagucucgcuggcagcagggaaacguguucucuugcuccgugaugcacgaggcucugcacaaucauuacacacagaaaucccugucucuguccccaggaaagugaccgugagcuga

SEQ ID NO: 34 - protein, 976 aa, Artificial Sequence
  Protein sequence encoding hsACE2-GS-Fc wherein the hsACE2 is hsACE2 N90D R273A
MSSSSWLLLSLVAVTAAQSTIEEQAKTFLDKFNHEAEDLFYQSSLASWNYNTNITEENVQNMNNAGDKWSAFLKEQSTLAQMYPLQEIQDLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 976 aa, Artificial Sequence
  Protein sequence encoding hsACE2-GS-Fc wherein the hsACE2 is hsACE2 N90D T92A
MSSSSWLLLSLVAVTAAQSTIEEQAKTFLDKFNHEAEDLFYQSSLASWNYNTNITEENVQNMNNAGDKWSAFLKEQSTLAQMYPLQEIQDLAVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK